(19) United States
(12) Patent Application Publication
(10) Pub. No.: US 2023/0357169 A1
(43) Pub. Date: Nov. 9, 2023

(54) ORGANIC COMPOUNDS, MIXTURES, AND USES THEREOF IN ORGANIC ELETRONIC DEVICES

(71) Applicant: Zhejiang Brilliant Optoelectronic Technology Co.,Ltd., Taizhou (CN)

(72) Inventors: Lei WEN, Taizhou (CN); Huaijun CHEN, Taizhou (CN); Junyou PAN, Taizhou (CN)

(73) Assignee: Zhejiang Brilliant Optoelectronic Technology Co.,Ltd., Taizhou (CN)

(21) Appl. No.: 18/351,664

(22) Filed: Jul. 13, 2023

Related U.S. Application Data
(63) Continuation of application No. PCT/CN2022/071815, filed on Jan. 13, 2022.

(30) Foreign Application Priority Data
Jan. 13, 2021 (CN) 202110041373.3

(51) Int. Cl. C07D 498/06; C07D 251/22; C07D 513/06; H10K 85/60; C07D 239/26; C07D 213/53; H10K 85/30; H10K 85/40

(52) U.S. Cl. CPC C07D 251/22; C07D 213/53; C07D 239/26; C07D 498/06; C07D 513/06; H10K 85/342; H10K 85/346; H10K 85/40; H10K 85/615; H10K 85/622; H10K 85/631; H10K 85/654; H10K 85/6572; H10K 85/6574; H10K 85/6576; H10K 85/658; C07B 2200/05; H10K 2101/10

(57) ABSTRACT

Provided are organic compounds including a structure of formula (I). Also provided are mixtures containing the organic compounds. Further provided are organic electronic devices containing the organic compounds or mixtures.

### CROSS-REFERENCE TO RELATED APPLICATIONS

The present application is a continuation of International Application No. PCT/CN2022/071815, filed on Jan. 13, 2022, which claims priority to Chinese Patent Application No. 202110041373.3, filed on Jan. 13, 2021. All of the aforementioned applications are incorporated herein by reference in their entireties.

### TECHINAL FIELD

The present disclosure relates to the technical field of organic electroluminescence, and in particular to an organic compound, a mixture, and an organic electronic device thereof.

### BACKGROUND

Due to the diversity of synthesis, low manufacturing cost, excellent optical, and electrical properties, organic light-emitting diodes (OLEDs) have great potential for the realization of novel optoelectronic devices, such as in flat-panel displays and lighting applications.

The organic electroluminescent phenomenon refers to a phenomenon of converting electrical energy to photonic energy with organic substance. An organic electroluminescent element utilizing the organic electroluminescent phenomenon usually has a structure comprising an anode, a cathode, and an organic layer therebetween. In order to improve the efficiency and lifetime of the organic electroluminescent element, the organic layer has a multi-layer structure, and each layer comprises different organic substances. For example, each layer can be a hole-injection layer, a hole-transport layer, a light-emitting layer, an electron-transport layer, an electron-injection layer, etc. When a voltage is applied between two electrodes of such an organic electroluminescent element, holes are injected into the organic layer from the anode, electrons are injected into an organic layer from the cathode; and an exciton is formed when an injected hole and an injected electron recombine in the emission layer. The exciton emits light when it transitions back to the ground state. The organic electroluminescent element has characteristics of self-emission, high luminance, high efficiency, low driving voltage, wide viewing angle, high contrast, high responsivity, etc.

Theories and experiments have confirmed that the emitting material is the most important factor affecting the efficiency of OLEDs. At present, the light-emitting layer of the organic electroluminescent element usually uses the host & dopant hybrid system as a light-emitting material to improve color purity, luminescence efficiency, and stability. In general, the selection of the host material is critical for the host & dopant hybrid system, because the host material greatly affects the efficiency and stability of OLEDs. Preferably, the host material should have a suitable molecular weight for the vacuum-deposition, a high glass transition temperature and a high thermal decomposition temperature for the good thermal stability, a high electrochemical stability for a long operation lifetime, and it should be easy to form an amorphous film, leading to a good interfacial interaction with the adjacent functional layer and less molecular movement.

Particular in the red-emitting host material, the red-emitting host material is required to have good carrier transport capability and suitable triplet energy level to ensure the energy can be effectively transferred to the dopant material during the light-emitting process, thereby achieving high efficiency. The currently reported red-emitting hosts usually have large conjugated aromatic system, such as carbazole derivates with fused-ring structures reported in WO2012169821, WO2012165844, WO2016013817, and still have the problem of the low device efficiency. Recently, CN110872300 reported the azidoheptadecazole structure, which disclosed the compound linked to aromatic group, but the device efficiency is still low.

The above-reported compounds suffer from the problems of low device efficiency and poor stability, while neglecting the carrier transport balance of the host material in the device. After an in-depth study by the present applicant, it is found that the device efficiency can be significantly improved by using large conjugated aromatic system linked to azole electron-absorbing structural unit, while the carrier transport balance can be further improved by using a suitable p-type material paired with it.

### SUMMARY

In one aspect, the present disclosure provides an organic compound comprising a structure of formula (I):

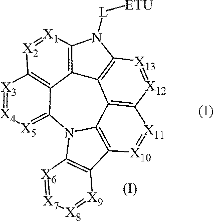

Where each of X₁ to X₁₃ independently represents CR₁, and the adjacent two CR_{1S} can be fused to form a ring;
ETU is a structure of formula (I-1) or (I-2);

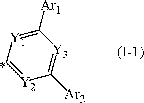

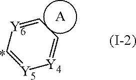

each “*” represents a site connecting L;
A is a C₆-C₃₀ aromatic ring, a C₅-C₃₀ heteroaromatic ring, or any combination thereof;
each of Y₁ to Y₆ independently represents N or CR₂, at least one of Y₁ to Y₃ is N, and at least one of Y₄ to Y₆ is N;
each of R₁ to R₂ at each occurrence is a substituent, and is independently selected from the group consisting of -H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, a CF₃ group, Cl, Br, F, a cross-linkable group, a substituted/unsubstituted aromatic or heteroaromatic group containing 5 to 40 ring atoms, an aryloxy or heteroaryloxy group containing 5 to 40 ring atoms, and any combination thereof;
L is a linking group selected from the group consisting of a single bond, a C₆-C₆₀ arylene group, a C₂-C₆₀ heteroaromatic group, a C₃-C₆₀ aliphatic ring, a C₆-C₆₀ fused aromatic ring, and any combination thereof;
each of Ar₁ and Ar₂ is independently selected from the group consisting of a substituted/unsubstituted aromatic or heteroaromatic group containing 5 to 40 ring atoms, an aryloxy or heteroaryloxy group containing 5 to 40 ring atoms, and any combination thereof, where one or more Ar₁- Ar₂ may form a monocyclic or polycyclic aliphatic or aromatic ring system with each other and/or with the rings bonded thereto.

In another aspect, the present disclosure also provides a polymer comprising at least one repeating unit, at least one of the repeating unit comprises a structure corresponding to an organic compound as described herein.

In yet another aspect, the present disclosure further provides a mixture comprising an organic compound H1 and an organic compound H2; the organic compound H1 is selected from an organic compound or a polymer as described herein; the organic compound H2 is another organic functional material, and is selected from one or more of the following: a hole-injection material, a hole-transport material, an electron-transport material, an electron-injection material, an electron-blocking material, a hole-blocking material, an emitting material, a host material, an organic dye.

In yet another aspect, the present disclosure further provides a formulation comprising at least one organic compound, or a polymer, or a mixture as described herein, and at least one organic solvent.

In yet another aspect, the present disclosure further provides an organic electronic device comprising a functional layer, the functional layer comprises an organic compound, or a polymer, or a mixture as described herein.

Beneficial effects: the use of the organic compounds as described herein in OLEDs, particularly in light-emitting layer, can provide superior device performance. Possible reasons therefor are as follows, but not limited thereto, the organic compounds as described herein have more balanced charge carrier transport property, which are conducive to improve the efficiency and lifetime of the related materials and devices.

### DETAILED DESCRIPTION OF THE EMBODIMENTS

The present disclosure provides an organic compound, a mixture, and the applications thereof in organic electronic devices, aiming to solve the problems of efficiency and lifetime of the existing OLEDs. In order to make the objects, technical solutions, and effects of the present disclosure more clear and definite, the present disclosure is further described in detail below. It should be understood that the embodiments described herein are only intended to explain the present disclosure, and are not intended to limit the present disclosure.

As used herein, the terms “formulation”, “printing ink”, and “ink” have the same meaning, and they are interchangeable with each other.

As used herein, the terms “host material”, “matrix material” have the same meaning, and they are interchangeable with each other.

As used herein, the term “substituted” means that a hydrogen atom of the compound is substituted.

As used herein, the term “the number of ring atoms” means that the number of atoms constituting the ring itself of a structural compound (e. g., a monocyclic compound, a fused ring compound, a cross-linked compound, a carbocyclic compound, and a heterocyclic compound) by covalent bonding. When the ring is substituted with a substituent, the atoms contained in the substituent are not included in the ring atoms. The above rule applies for all cases without further specific description. For example, the number of ring atoms of a benzene ring is 6, the number of ring atoms of a naphthalene ring is 10, and the number of ring atoms of a thienyl group is 5.

In the embodiments of the present disclosure, the energy level structure of the organic material, the singlet energy level E_{S1}, triplet energy level E_{T1}, HOMO, and LUMO play key roles. The determination of these energy levels is introduced as follows.

HOMO and LUMO energy levels can be measured by photoelectric effects, for example by XPS (X-ray photoelectron spectroscopy), UPS (UV photoelectron spectroscopy), or by cyclic voltammetry (hereinafter referred to as CV). Recently, quantum chemical methods, such as density functional theory (hereinafter referred to as DFT), are becoming effective methods for calculating the molecular orbital energy levels.

The singlet energy level E_{S1} of the organic material can be determined by the emission spectrum, and the triplet energy level E_{T1} can be measured by a low-temperature time-resolved spectroscopy. E_{S1} and E_{T1} can also be obtained by quantum simulation calculation (for example, by Time-dependent DFT), for instance with the commercial software Gaussian 09W (Gaussian Inc.). The specific simulation method can be found in WO2011141110 or as described in the examples below. ΔE_{ST} is defined as (E_{S1}-E_{T1}).

It should be noted that the absolute values of HOMO, LUMO, E_{S1}, and E_{T1} may vary depending on the measurement method or calculation method used. Even for the same method, different ways of evaluation, for example, using either the onset or peak value of a CV curve as reference, may result in different (HOMO/LUMO) values. Therefore, reasonable and meaningful comparison should be carried out by using the same measurement and evaluation methods. In the embodiments of the present disclosure, the values of HOMO, LUMO, E_{S1}, and E_{T1} are based on Time-dependent DFT simulation, which however should not exclude the applications of other measurement or calculation methods.

In the disclosure, (HOMO-1) is defined as the second highest occupied orbital level, (HOMO-2) is defined as the third highest occupied orbital level, and so on. (LUMO+1) is defined as the second lowest unoccupied orbital level, (LUMO+2) is defined as the third lowest occupied orbital level, and so on.

In one aspect, the present disclosure provides an organic compound comprising a structure of formula (I):

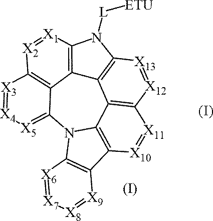

Where each of X₁ to X₁₃ independently represents CR₁, and the adjacent two CR₁s can be fused to form a ring;
ETU is a structure of formula (I-1) or (I-2);

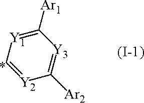

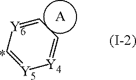

“*” represents a site connecting L; A is a C₆-C₃₀ aromatic ring, a C₅-C₃₀ heteroaromatic ring, or any combination thereof; each of Y₁ to Y₆ independently represents N or CR₂, at least one of Y₁ to Y₃ is N, and at least one of Y₄ to Y₆ is N;
each of R₁ to R₂ at each occurrence is independently selected from the group consisting ofH, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, a CF₃ group, Cl, Br, F, a cross-linkable group, a substituted/unsubstituted aromatic or heteroaromatic group containing 5 to 40 ring atoms, an aryloxy or heteroaryloxy group containing 5 to 40 ring atoms, and any combination thereof;
L is a linking group selected from the group consisting of a single bond, a C₆-C₆₀ arylene group, a C₂-C₆₀ heteroaromatic group, a C₃-C₆₀ aliphatic ring, a C₆-C₆₀ fused aromatic ring, and any combination thereof;
each of Ar₁ and Ar₂ is independently selected from the group consisting of a substituted/unsubstituted aromatic/heteroaromatic group containing 5 to 40 ring atoms, an aryloxy or heteroaryloxy group containing 5 to 40 ring atoms, and any combination thereof, where one or more Ar₁-Ar₂ may form a monocyclic or polycyclic aliphatic or aromatic ring system with each other and/or with the rings bonded thereto.

In some embodiments, each R₁ and R₂ at each occurrence is independently selected from the group consisting of -H, -D, a cyano group, a C₁-C₁₈ linear alkyl group, a C₃-C₁₈ branched/cyclic alkyl group, a C₃-C₁₈ branched/cyclic alkoxy group, a C₃-C₁₈ branched/cyclic thioalkoxy group, a C₃-C₁₈ branched/cyclic silyl group, and a C₅-C₃₀ substituted/unsubstituted aryl or heteroaryl or aryloxy or heteroaryloxy group. In some embodiments, each of R₁ to R₂ at each occurrence is independently selected from the group consisting of -D, a C₁-C₁₂ linear alkyl group, and a C₅-C₂₀ substituted/unsubstituted aryl or heteroaryl or aryloxy or heteroaryloxy group. In some embodiments, each of R₁ to R₂ at each occurrence is independently selected from -D, a C₁-C₆ linear alkyl group, and a C₅-C₁₅ substituted/unsubstituted aryl or heteroaryl or aryloxy or heteroaryloxy group.

In some embodiments, R₁ and R₂ at each occurrence may be fully deuterated or partially deuterated, respectively.

In some embodiments, formula (I) is selected from a structure of formula (II-1) or (II-2):

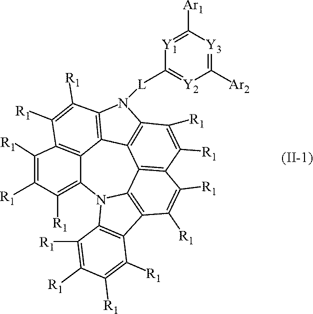

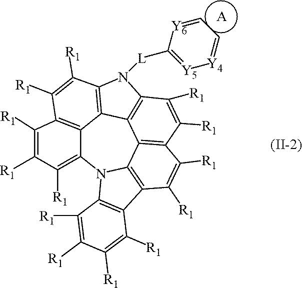

Where Ar₁, Ar₂, R₁, L, Y₁-Y₆, A are identically defined as described above.

In some embodiments, the adjacent two R₁s of formula (II-1) or (II-2) may be fused to form a ring.

In some embodiments, ETU of formula (I) is selected from the following groups:

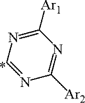

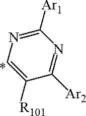

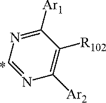

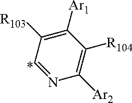

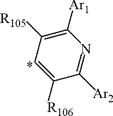

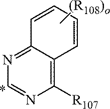

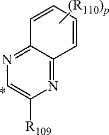

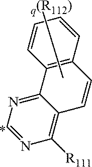

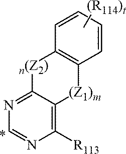

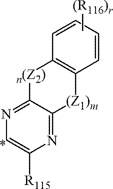

Where each of Z₁ and Z₂ is independently selected from S, O, CR₁₁₇R₁₁₈, or NR₁₁₉; each of R₁₀₁ to R₁₁₉ can be the same or different groups with the same meaning as the above-mentioned R₁; o, p, t, r are integers from 0 to 4; q is integer from 0 to 6; each of m and n is 0 or 1, and m+n=1; Ar₁ to Ar₂, “*” are the same as those defined above.

In some embodiments, ETU is selected from the following groups:

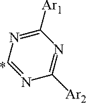

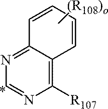

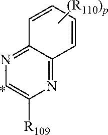

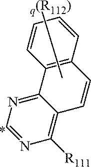

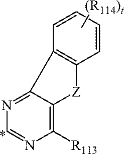

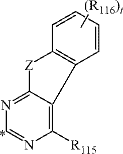

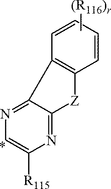

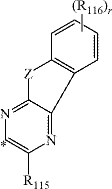

Where Ar₁, Ar₂, “*”, R₁₀₇-R₁₁₆, o, p, t, r are identically defined as described above.

In some embodiments, Ar₁ to Ar₂, and L of the organic compound are independently selected from one or combinations of more than one of the following groups:

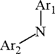

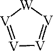

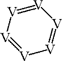

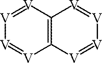

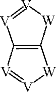

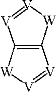

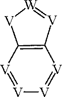

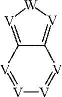

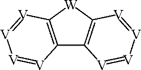

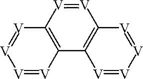

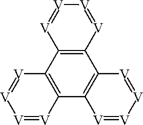

Where V in multiple occurrences, is independently selected from CR₃ or N; W in multiple occurrences, is independently selected from CR₄R₅, SiR₆R₇, NR₈, C(═O), S, or O.

Each of Ar₁ and Ar₂ is independently selected from the group consisting of a C₅-C₄₀ substituted/unsubstituted aromatic or heteroaromatic group, a C₅-C₄₀ aryloxy or heteroaryloxy group, and any combination thereof; each of R₃ to R₈ is a substituent, and is independently selected from the group consisting of -H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, a CF₃ group, Cl, Br, F, a cross-linkable group, a substituted/unsubstituted aromatic or heteroaromatic group containing 5 to 40 ring atoms, an aryloxy or heteroaryloxy group containing 5 to 40 ring atoms, and any combination thereof, where one or more R₃-R₈ may form a ring system with each other and/or with the ring bonded thereto.

Particularly, each of R₃ to R₈ at each occurrence is independently selected from the group consisting of -D, a cyano group, a C₁-C₁₈ linear alkyl group, a C₃-C₁₈ branched/cyclic alkyl group, a C₃-C₁₈ branched/cyclic alkoxy group, a C₃-C₁₈ branched/cyclic thioalkoxy group, a C₃-C₁₈ branched/cyclic silyl group, and a C₅-C₃₀ substituted/unsubstituted aryl or heteroaryl or aryloxy or heteroaryloxy group. In some embodiments, each of R₃ to R₈ at each occurrence is independently selected from the group consisting of -D, a C₁-C₁₂ linear alkyl group, and a C₅-C₂₀ substituted/unsubstituted aryl or heteroaryl or aryloxy or heteroaryloxy group.

In some embodiments, each of R₃ to R₈ at each occurrence is independently selected from -D, a

C₁-C₆ linear alkyl group, and a C₅-C₁₅ substituted/unsubstituted aryl or heteroaryl or aryloxy or heteroaryloxy group.

In some embodiments, Ar₁ to Ar₂, and L of the organic compound are independently selected from one or combinations of more than one of the following groups:

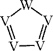

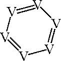

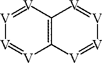

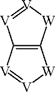

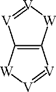

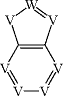

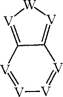

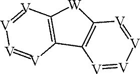

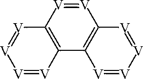

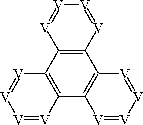

Where W, V are the same as those defined above.

In some embodiments, L is biphenyl, or a structure in which one or more carbon atoms in biphenyl are substituted with N atoms.

In some embodiments, L is benzene, or a structure in which one or more carbon atoms in benzene are substituted with N atoms.

In some embodiments, L of the organic compound is preferably selected from combinations of one or more structures of the following formulas in which the H atom on the ring can be arbitrarily substituted:

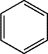

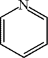

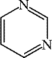

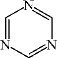

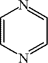

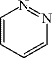

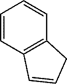

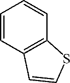

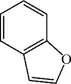

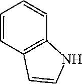

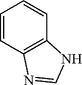

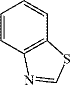

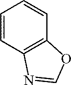

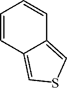

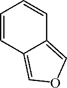

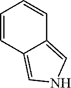

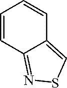

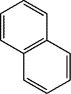

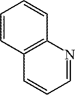

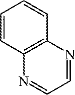

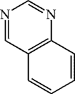

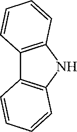 [SMILES: c1ccc2c(c1)[nH]c1ccccc12]

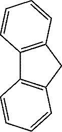 [SMILES: c1ccc2c(c1)Cc1ccccc1-2]

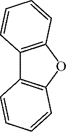 [SMILES: c1ccc2c(c1)oc1ccccc12]

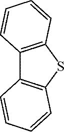 [SMILES: c1ccc2c(c1)sc1ccccc12]

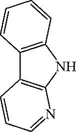 [SMILES: c1ccc2c(c1)[nH]c1ncccc12]

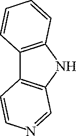 [SMILES: c1ccc2c(c1)[nH]c1cnccc12]

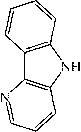 [SMILES: c1ccc2c(c1)[nH]c1cccnc12]

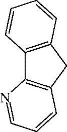 [SMILES: c1ccc2c(c1)Cc1cccnc1-2]

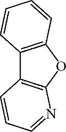 [SMILES: c1ccc2c(c1)oc1ncccc12]

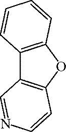 [SMILES: c1ccc2c(c1)oc1ccncc12]

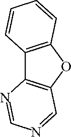 [SMILES: c1ccc2c(c1)oc1cncnc12]

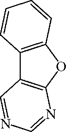 [SMILES: c1ccc2c(c1)oc1ncncc12]

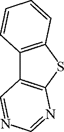 [SMILES: c1ccc2c(c1)sc1ncncc12]

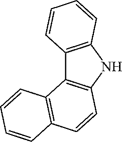 [SMILES: c1ccc2c(c1)ccc1[nH]c3ccccc3c12]

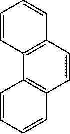 [SMILES: c1ccc2c(c1)ccc1ccccc12]

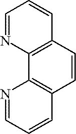 [SMILES: c1cnc2c(c1)ccc1cccnc12]

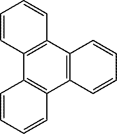 [SMILES: c1ccc2c(c1)c1ccccc1c1ccccc21]

In some embodiments, L of the organic compound may comprise a structure of the following formulas in which the H atom on the ring can be arbitrarily substituted:

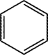

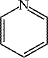

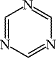

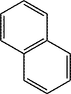

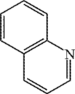

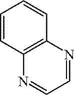

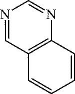

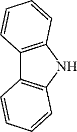 [SMILES: c1ccc2c(c1)[nH]c1ccccc12]

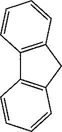 [SMILES: c1ccc2c(c1)Cc1ccccc1-2]

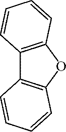 [SMILES: c1ccc2c(c1)oc1ccccc12]

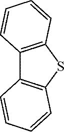 [SMILES: c1ccc2c(c1)sc1ccccc12]

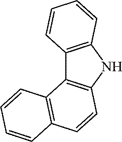 [SMILES: c1ccc2c(c1)ccc1[nH]c3ccccc3c12]

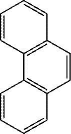 [SMILES: c1ccc2c(c1)ccc1ccccc12]

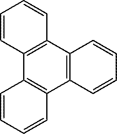 [SMILES: c1ccc2c(c1)c1ccccc1c1ccccc21]

each of Ar₁ and Ar₂ at each occurrence is independently selected from the group consisting of a C₅-C₄₀ substituted/unsubstituted aromatic or heteroaromatic or aryloxy or heteroaryloxy group, and any combination thereof, where one or more Ar₁-Ar₂ may form a polycyclic aliphatic or aromatic ring system with each other and/or with the rings bonded thereto.

In some embodiments, Ar may be selected from the group consisting of a C₅-C₂₀ deuterated/undeuterated substituted/unsubstituted aromatic or heteroaromatic group, a C₅-C₂₀ deuterated/undeuterated aryloxy or heteroaryloxy group, and any combination thereof, where one or more Ar₁-Ar₂ may form a monocyclic or polycyclic aliphatic or aromatic ring system with each other and/or with the rings bonded thereto.

In some embodiments, Ar may be selected from the group consisting of a C₅-C₁₅ deuterated/undeuterated substituted/unsubstituted aromatic or heteroaromatic group, a C₅-C₁₅ deuterated/undeuterated aryloxy or heteroaryloxy group, and any combination thereof, where one or more Ar₁-Ar₂ may form a monocyclic or polycyclic aliphatic or aromatic ring system with each other and/or with the ring bonded thereto.

In some embodiments, Ar is selected from benzene, naphthalene, phenanthrene, triphenylene, dibiphenyl, terphenyl, or a structure in which one or more carbon atoms of these structures are substituted with N atoms.

In the present disclosure, the term “aromatic group” refers to a hydrocarbon group consisting of an aromatic ring, including monocyclic groups and polycyclic systems. The term “heteroaromatic group” refers to a heteroaromatic group consisting of at least one heteroaromatic ring, including monocyclic groups and polycyclic systems. The polycyclic systems contain two or more rings, in which two carbon atoms are shared by two adjacent rings, i.e. fused rings. Specifically, at least one of the rings in the polycyclic rings are aromatic or heteroaromatic. For the purposes of the present disclosure, the aromatic ring groups or heteroaromatic groups comprise not only aromatic or heteroaromatic systems, but also a plurality of aromatic or heteroaromatic groups are interconnected by short non-aromatic units (for example C, N, O, Si, S, P atoms). Therefore, systems such as 9,9′-spirobifluorene, 9,9-diaryl fluorene, triarylamine, diaryl ethers, and other systems, should also be considered as aromatic groups for the purpose of this disclosure.

Specifically, examples of the aromatic groups include benzene, naphthalene, anthracene, phenanthrene, perylene, tetracene, pyrene, benzopyrene, triphenylene, acenaphthylene, fluorene, and derivatives thereof.

Specifically, examples of heteroaromatic groups include: furan, benzofuran, thiophene, benzothiophene, pyrrole, pyrazole, triazole, imidazole, oxazole, oxadiazole, thiazole, tetrazole, indole, carbazole, pyrroloimidazole, pyrrolopyrrole, thienopyrrole, thienothiophene, furopyrrole, furofuran, thienofuran, benzisoxazole, benzisothiazole, benzimidazole, pyridine, pyrazine, pyridazine, pyrimidine, triazine, quinoline, isoquinoline, o-diazonaphthalene, quinoxaline, phenanthridine, pyrimidine, quinazoline, quinazolinone, and derivatives thereof.

In some embodiments, the organic compound as described herein has electron-transport property.

In some embodiments, Ar₁ and/or Ar₂ of the organic compound comprises an electron-accepting group, or is substituted with an electron-accepting group. The suitable electron-accepting groups may be independently selected from F, a cyano group, or one of the following groups:

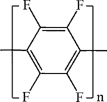

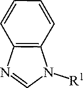

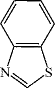

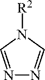

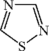

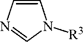

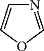

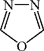

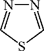

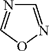

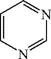

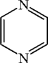

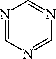

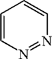

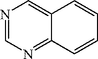

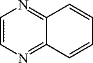

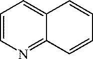

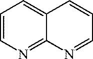

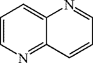

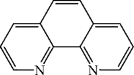

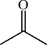

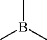

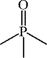

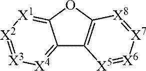

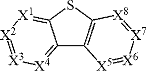

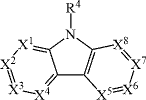

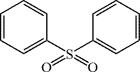

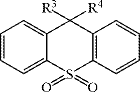

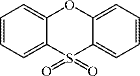

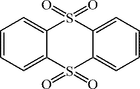

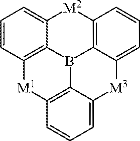

Where n is an integer from 1 to 3; each of X¹ to X⁸ is CR⁵ or N, and at least one is N. Meanwhile, R⁵ at any two adjacent positions can be a monocyclic or polycyclic aliphatic or aromatic ring system; M¹, M², M³ independently represent N(R⁶), C(R⁷)₂, Si(R⁸)₂, O, C═N(R⁹), C═C(R¹⁰)₂, P(R¹¹), P(═O)R¹², S, S═O, SO₂, or null; R¹ to R¹² are identically defined as the above-mentioned R₁.

In some embodiments, the electron-accepting group of the organic compound is selected from combinations of one or more following groups:

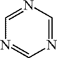

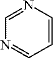

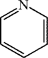

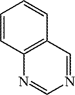

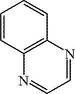

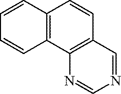 [SMILES: c1ccc2c(c1)ccc1cncnc12]

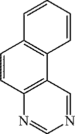 [SMILES: c1ccc2c(c1)ccc1ncncc12]

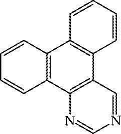 [SMILES: c1ccc2c(c1)c1ccccc1c1ncncc21]

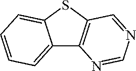 [SMILES: c1ccc2c(c1)sc1cncnc12]

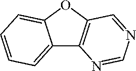 [SMILES: c1ccc2c(c1)oc1cncnc12]

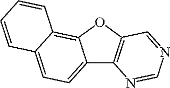 [SMILES: c1ccc2c(c1)ccc1c3ncncc3oc21]

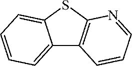 [SMILES: c1ccc2c(c1)sc1ncccc12]

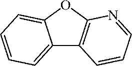 [SMILES: c1ccc2c(c1)oc1ncccc12]

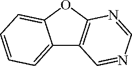 [SMILES: c1ccc2c(c1)oc1ncncc12]

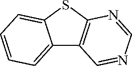 [SMILES: c1ccc2c(c1)sc1ncncc12]

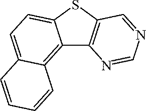 [SMILES: c1ccc2c(c1)ccc1sc3cncnc3c12]

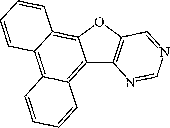 [SMILES: c1ccc2c(c1)c1ccccc1c1c3ncncc3oc21]

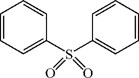 [SMILES: O=S(=O)(c1ccccc1)c1ccccc1]

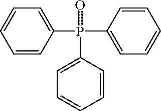 [SMILES: O=P(c1ccccc1)(c1ccccc1)c1ccccc1]

In some embodiments, the organic compound as described herein has a small singlet-triplet energy level difference (ΔEₛₜ), typically ΔEₛₜ ≤0.3 eV, preferably ΔEₛₜ ≤0.2 eV, more preferably ΔEₛₜ ≤0.15 eV, and most preferably ΔEₛₜ ≤0.10 eV.

Specific examples of the organic compounds of formula (I) as described herein are listed below, but not limited thereto.

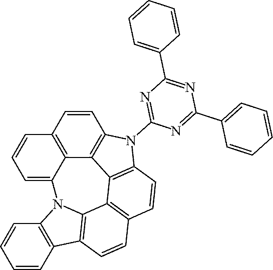 [SMILES: c1ccc(-c2nc(-c3ccccc3)nc(-n3c4ccc5cccc6c5c4c4c5c(ccc7c8ccccc8n6c75)ccc43)n2)cc1]

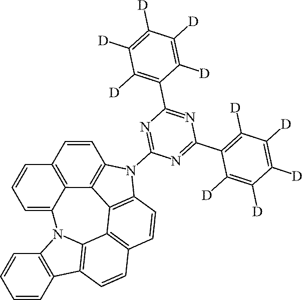 [SMILES: [2H]c1c([2H])c([2H])c(-c2nc(-c3c([2H])c([2H])c([2H])c([2H])c3[2H])nc(-n3c4ccc5cccc6c5c4c4c5c(ccc7c8ccccc8n6c75)ccc43)n2)c([2H])c1[2H]]

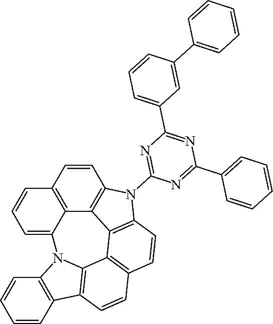 [SMILES: c1ccc(-c2cccc(-c3nc(-c4ccccc4)nc(-n4c5ccc6cccc7c6c5c5c6c(ccc8c9ccccc9n7c86)ccc54)n3)c2)cc1]

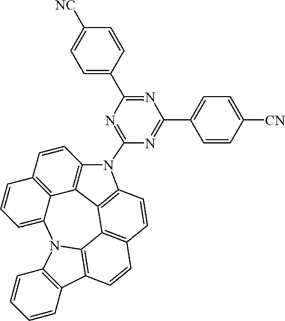

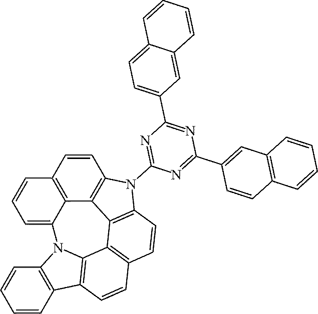 [SMILES: c1ccc2cc(-c3nc(-c4ccc5ccccc5c4)nc(-n4c5ccc6cccc7c6c5c5c6c(ccc8c9ccccc9n7c86)ccc54)n3)ccc2c1]

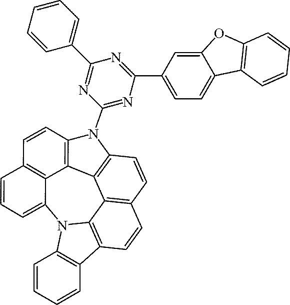 [SMILES: c1ccc(-c2nc(-c3ccc4c(c3)oc3ccccc34)nc(-n3c4ccc5cccc6c5c4c4c5c(ccc7c8ccccc8n6c75)ccc43)n2)cc1]

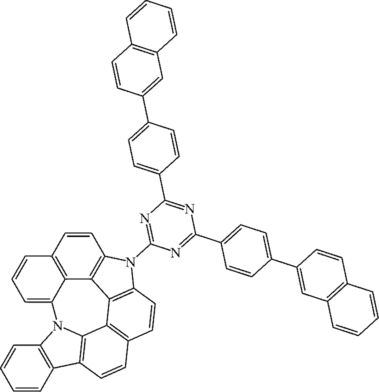 [SMILES: c1ccc2cc(-c3ccc(-c4nc(-c5ccc(-c6ccc7ccccc7c6)cc5)nc(-n5c6ccc7cccc8c7c6c6c7c(ccc9c%10ccccc%10n8c97)ccc65)n4)cc3)ccc2c1]

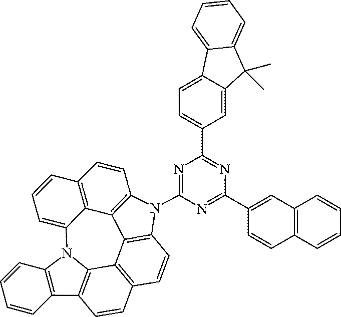 [SMILES: CC1(C)c2ccccc2-c2ccc(-c3nc(-c4ccc5ccccc5c4)nc(-n4c5ccc6cccc7c6c5c5c6c(ccc8c9ccccc9n7c86)ccc54)n3)cc21]

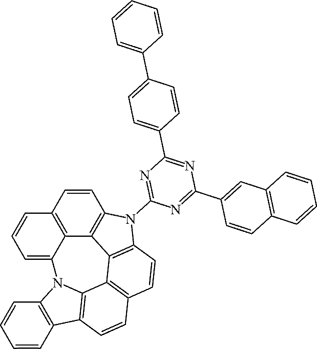 [SMILES: c1ccc(-c2ccc(-c3nc(-c4ccc5ccccc5c4)nc(-n4c5ccc6cccc7c6c5c5c6c(ccc8c9ccccc9n7c86)ccc54)n3)cc2)cc1]

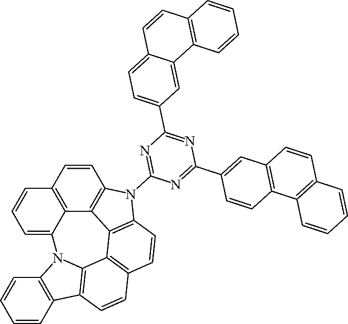 [SMILES: c1ccc2c(c1)ccc1cc(-c3nc(-c4ccc5ccc6ccccc6c5c4)nc(-n4c5ccc6cccc7c6c5c5c6c(ccc8c9ccccc9n7c86)ccc54)n3)ccc12]

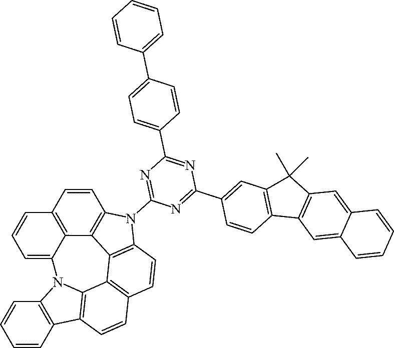 [SMILES: CC1(C)c2cc(-c3nc(-c4ccc(-c5ccccc5)cc4)nc(-n4c5ccc6cccc7c6c5c5c6c(ccc8c9ccccc9n7c86)ccc54)n3)ccc2-c2cc3ccccc3cc21]

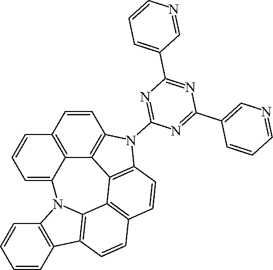 [SMILES: c1cncc(-c2nc(-c3cccnc3)nc(-n3c4ccc5cccc6c5c4c4c5c(ccc7c8ccccc8n6c75)ccc43)n2)c1]

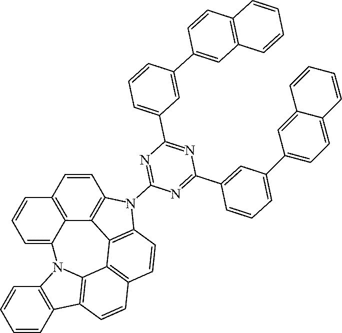 [SMILES: c1cc(-c2ccc3ccccc3c2)cc(-c2nc(-c3cccc(-c4ccc5ccccc5c4)c3)nc(-n3c4ccc5cccc6c5c4c4c5c(ccc7c8ccccc8n6c75)ccc43)n2)c1]

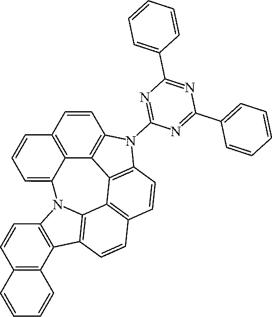 [SMILES: c1ccc(-c2nc(-c3ccccc3)nc(-n3c4ccc5cccc6c5c4c4c5c(ccc7c8c9ccccc9ccc8n6c75)ccc43)n2)cc1]

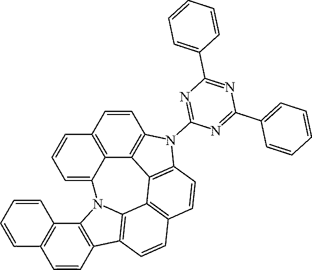 [SMILES: c1ccc(-c2nc(-c3ccccc3)nc(-n3c4ccc5cccc6c5c4c4c5c(ccc7c8ccc9ccccc9c8n6c75)ccc43)n2)cc1]

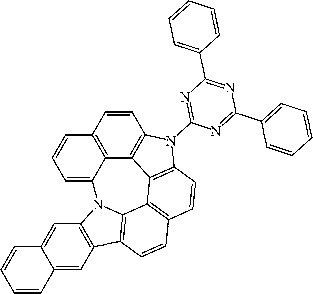 [SMILES: c1ccc(-c2nc(-c3ccccc3)nc(-n3c4ccc5cccc6c5c4c4c5c(ccc7c8cc9ccccc9cc8n6c75)ccc43)n2)cc1]

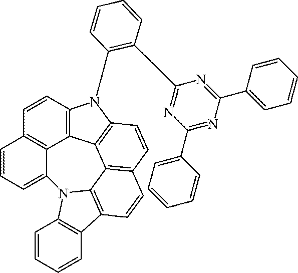 [SMILES: c1ccc(-c2nc(-c3ccccc3)nc(-c3ccccc3-n3c4ccc5cccc6c5c4c4c5c(ccc7c8ccccc8n6c75)ccc43)n2)cc1]

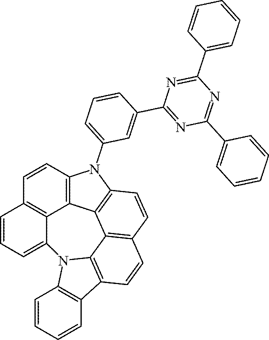 [SMILES: c1ccc(-c2nc(-c3ccccc3)nc(-c3cccc(-n4c5ccc6cccc7c6c5c5c6c(ccc8c9ccccc9n7c86)ccc54)c3)n2)cc1]

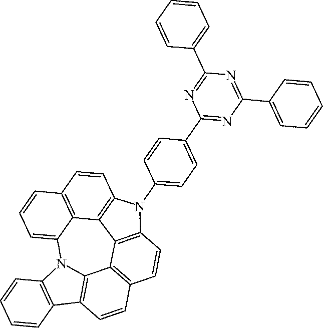 [SMILES: c1ccc(-c2nc(-c3ccccc3)nc(-c3ccc(-n4c5ccc6cccc7c6c5c5c6c(ccc8c9ccccc9n7c86)ccc54)cc3)n2)cc1]

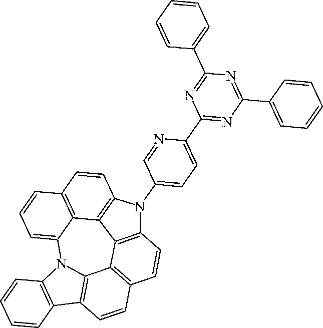 [SMILES: c1ccc(-c2nc(-c3ccccc3)nc(-c3ccc(-n4c5ccc6cccc7c6c5c5c6c(ccc8c9ccccc9n7c86)ccc54)cn3)n2)cc1]

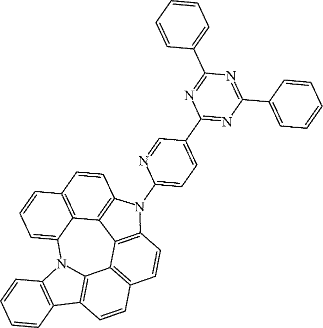 [SMILES: c1ccc(-c2nc(-c3ccccc3)nc(-c3ccc(-n4c5ccc6cccc7c6c5c5c6c(ccc8c9ccccc9n7c86)ccc54)nc3)n2)cc1]

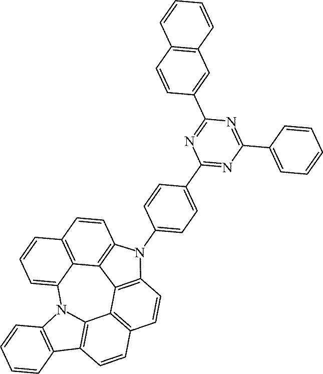 [SMILES: c1ccc(-c2nc(-c3ccc(-n4c5ccc6cccc7c6c5c5c6c(ccc8c9ccccc9n7c86)ccc54)cc3)nc(-c3ccc4ccccc4c3)n2)cc1]

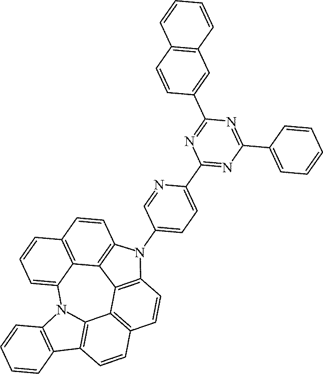 [SMILES: c1ccc(-c2nc(-c3ccc4ccccc4c3)nc(-c3ccc(-n4c5ccc6cccc7c6c5c5c6c(ccc8c9ccccc9n7c86)ccc54)cn3)n2)cc1]

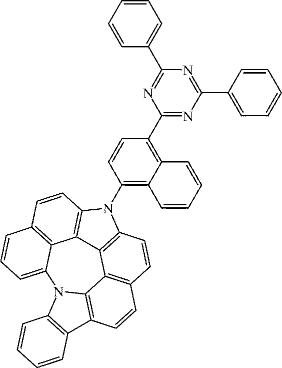 [SMILES: c1ccc(-c2nc(-c3ccccc3)nc(-c3ccc(-n4c5ccc6cccc7c6c5c5c6c(ccc8c9ccccc9n7c86)ccc54)c4ccccc34)n2)cc1]

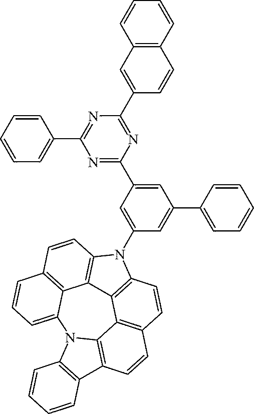 [SMILES: c1ccc(-c2cc(-c3nc(-c4ccccc4)nc(-c4ccc5ccccc5c4)n3)cc(-n3c4ccc5cccc6c5c4c4c5c(ccc7c8ccccc8n6c75)ccc43)c2)cc1]

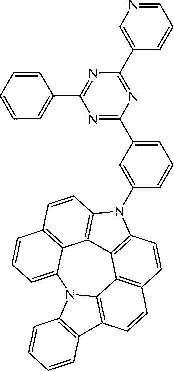 [SMILES: c1ccc(-c2nc(-c3cccnc3)nc(-c3cccc(-n4c5ccc6cccc7c6c5c5c6c(ccc8c9ccccc9n7c86)ccc54)c3)n2)cc1]

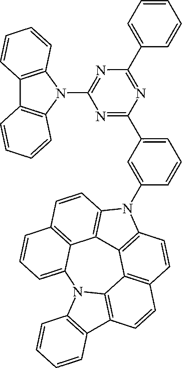 [SMILES: c1ccc(-c2nc(-c3cccc(-n4c5ccc6cccc7c6c5c5c6c(ccc8c9ccccc9n7c86)ccc54)c3)nc(-n3c4ccccc4c4ccccc43)n2)cc1]

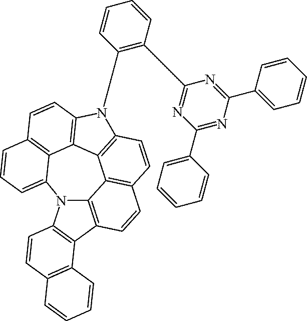 [SMILES: c1ccc(-c2nc(-c3ccccc3)nc(-c3ccccc3-n3c4ccc5cccc6c5c4c4c5c(ccc7c8c9ccccc9ccc8n6c75)ccc43)n2)cc1]

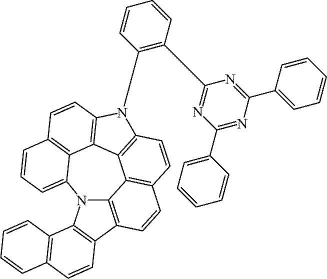 [SMILES: c1ccc(-c2nc(-c3ccccc3)nc(-c3ccccc3-n3c4ccc5cccc6c5c4c4c5c(ccc7c8ccc9ccccc9c8n6c75)ccc43)n2)cc1]

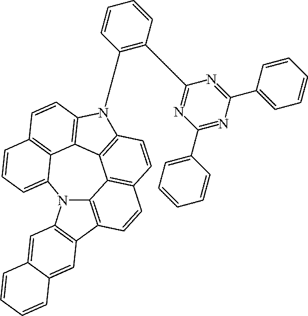 [SMILES: c1ccc(-c2nc(-c3ccccc3)nc(-c3ccccc3-n3c4ccc5cccc6c5c4c4c5c(ccc7c8cc9ccccc9cc8n6c75)ccc43)n2)cc1]

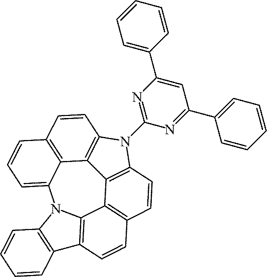 [SMILES: c1ccc(-c2cc(-c3ccccc3)nc(-n3c4ccc5cccc6c5c4c4c5c(ccc7c8ccccc8n6c75)ccc43)n2)cc1]

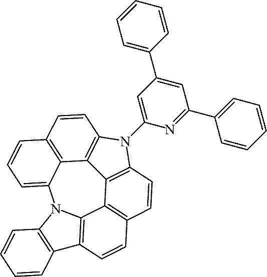 [SMILES: c1ccc(-c2cc(-c3ccccc3)nc(-n3c4ccc5cccc6c5c4c4c5c(ccc7c8ccccc8n6c75)ccc43)c2)cc1]

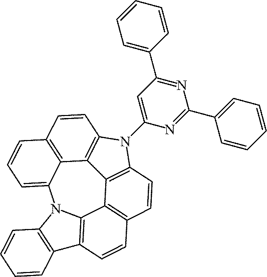 [SMILES: c1ccc(-c2cc(-n3c4ccc5cccc6c5c4c4c5c(ccc7c8ccccc8n6c75)ccc43)nc(-c3ccccc3)n2)cc1]

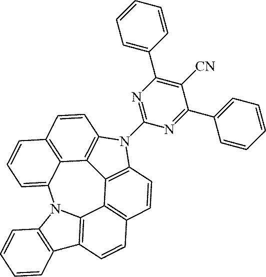 [SMILES: N#Cc1c(-c2ccccc2)nc(-n2c3ccc4cccc5c4c3c3c4c(ccc6c7ccccc7n5c64)ccc32)nc1-c1ccccc1]

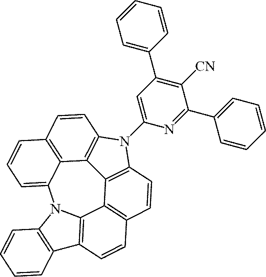 [SMILES: N#Cc1c(-c2ccccc2)cc(-n2c3ccc4cccc5c4c3c3c4c(ccc6c7ccccc7n5c64)ccc32)nc1-c1ccccc1]

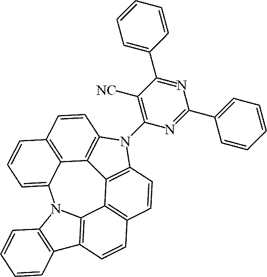

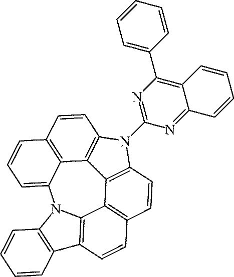 [SMILES: c1ccc(-c2nc(-n3c4ccc5cccc6c5c4c4c5c(ccc7c8ccccc8n6c75)ccc43)nc3ccccc23)cc1]

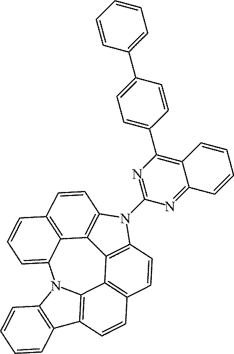 [SMILES: c1ccc(-c2ccc(-c3nc(-n4c5ccc6cccc7c6c5c5c6c(ccc8c9ccccc9n7c86)ccc54)nc4ccccc34)cc2)cc1]

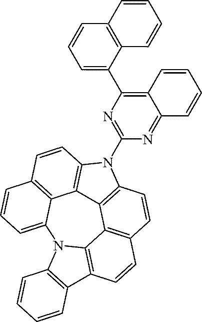 [SMILES: c1ccc2c(-c3nc(-n4c5ccc6cccc7c6c5c5c6c(ccc8c9ccccc9n7c86)ccc54)nc4ccccc34)cccc2c1]

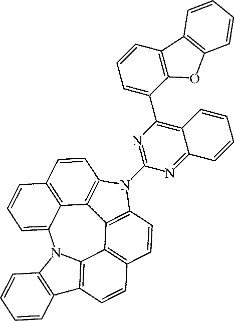 [SMILES: c1ccc2c(-c3cccc4c3oc3ccccc34)nc(-n3c4ccc5cccc6c5c4c4c5c(ccc7c8ccccc8n6c75)ccc43)nc2c1]

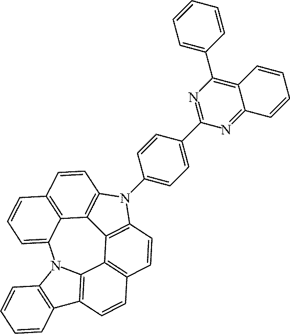 [SMILES: c1ccc(-c2nc(-c3ccc(-n4c5ccc6cccc7c6c5c5c6c(ccc8c9ccccc9n7c86)ccc54)cc3)nc3ccccc23)cc1]

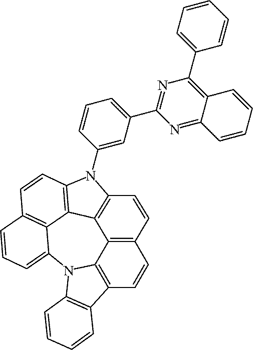 [SMILES: c1ccc(-c2nc(-c3cccc(-n4c5ccc6cccc7c6c5c5c6c(ccc8c9ccccc9n7c86)ccc54)c3)nc3ccccc23)cc1]

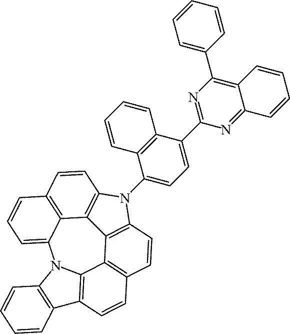 [SMILES: c1ccc(-c2nc(-c3ccc(-n4c5ccc6cccc7c6c5c5c6c(ccc8c9ccccc9n7c86)ccc54)c4ccccc34)nc3ccccc23)cc1]

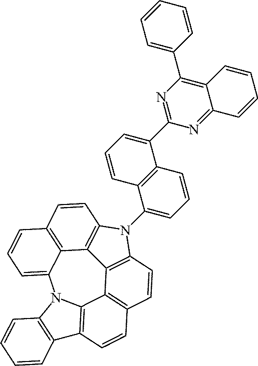 [SMILES: c1ccc(-c2nc(-c3cccc4c(-n5c6ccc7cccc8c7c6c6c7c(ccc9c%10ccccc%10n8c97)ccc65)cccc34)nc3ccccc23)cc1]

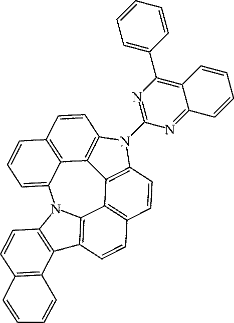 [SMILES: c1ccc(-c2nc(-n3c4ccc5cccc6c5c4c4c5c(ccc7c8c9ccccc9ccc8n6c75)ccc43)nc3ccccc23)cc1]

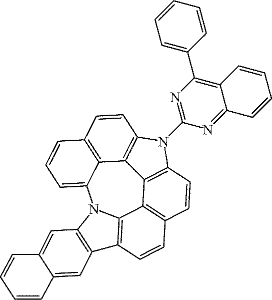 [SMILES: c1ccc(-c2nc(-n3c4ccc5cccc6c5c4c4c5c(ccc7c8cc9ccccc9cc8n6c75)ccc43)nc3ccccc23)cc1]

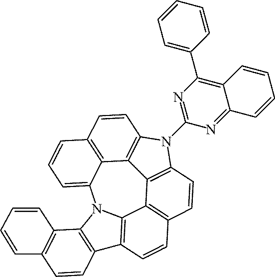 [SMILES: c1ccc(-c2nc(-n3c4ccc5cccc6c5c4c4c5c(ccc7c8ccc9ccccc9c8n6c75)ccc43)nc3ccccc23)cc1]

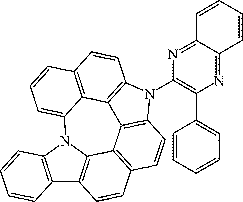 [SMILES: c1ccc(-c2nc3ccccc3nc2-n2c3ccc4cccc5c4c3c3c4c(ccc6c7ccccc7n5c64)ccc32)cc1]

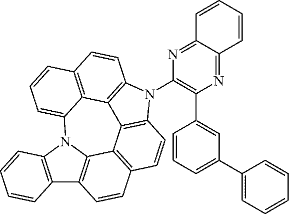 [SMILES: c1ccc(-c2cccc(-c3nc4ccccc4nc3-n3c4ccc5cccc6c5c4c4c5c(ccc7c8ccccc8n6c75)ccc43)c2)cc1]

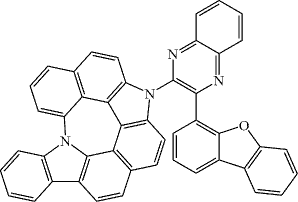 [SMILES: c1ccc2nc(-n3c4ccc5cccc6c5c4c4c5c(ccc7c8ccccc8n6c75)ccc43)c(-c3cccc4c3oc3ccccc34)nc2c1]

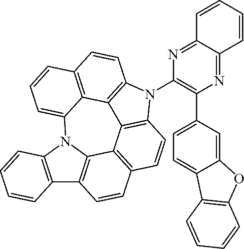 [SMILES: c1ccc2nc(-n3c4ccc5cccc6c5c4c4c5c(ccc7c8ccccc8n6c75)ccc43)c(-c3ccc4c(c3)oc3ccccc34)nc2c1]

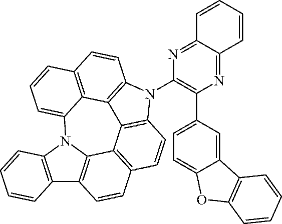 [SMILES: c1ccc2nc(-n3c4ccc5cccc6c5c4c4c5c(ccc7c8ccccc8n6c75)ccc43)c(-c3ccc4oc5ccccc5c4c3)nc2c1]

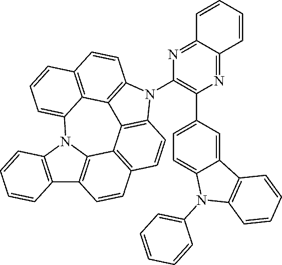 [SMILES: c1ccc(-n2c3ccccc3c3cc(-c4nc5ccccc5nc4-n4c5ccc6cccc7c6c5c5c6c(ccc8c9ccccc9n7c86)ccc54)ccc32)cc1]

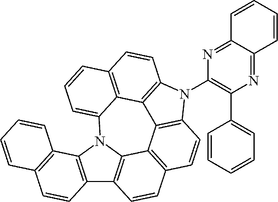 [SMILES: c1ccc(-c2nc3ccccc3nc2-n2c3ccc4cccc5c4c3c3c4c(ccc6c7ccc8ccccc8c7n5c64)ccc32)cc1]

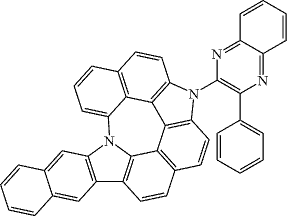 [SMILES: c1ccc(-c2nc3ccccc3nc2-n2c3ccc4cccc5c4c3c3c4c(ccc6c7cc8ccccc8cc7n5c64)ccc32)cc1]

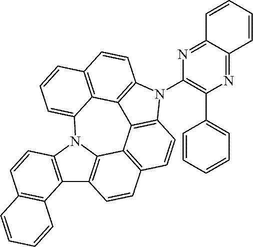 [SMILES: c1ccc(-c2nc3ccccc3nc2-n2c3ccc4cccc5c4c3c3c4c(ccc6c7c8ccccc8ccc7n5c64)ccc32)cc1]

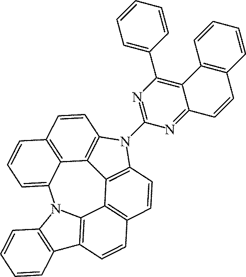 [SMILES: c1ccc(-c2nc(-n3c4ccc5cccc6c5c4c4c5c(ccc7c8ccccc8n6c75)ccc43)nc3ccc4ccccc4c23)cc1]

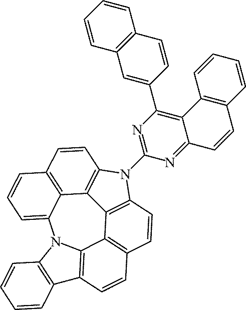 [SMILES: c1ccc2cc(-c3nc(-n4c5ccc6cccc7c6c5c5c6c(ccc8c9ccccc9n7c86)ccc54)nc4ccc5ccccc5c34)ccc2c1]

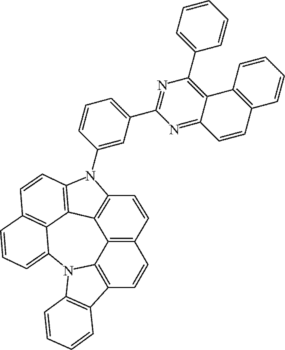 [SMILES: c1ccc(-c2nc(-c3cccc(-n4c5ccc6cccc7c6c5c5c6c(ccc8c9ccccc9n7c86)ccc54)c3)nc3ccc4ccccc4c23)cc1]

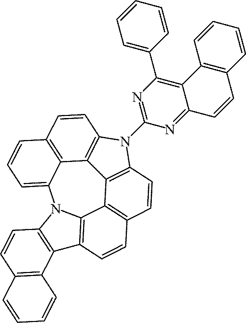 [SMILES: c1ccc(-c2nc(-n3c4ccc5cccc6c5c4c4c5c(ccc7c8c9ccccc9ccc8n6c75)ccc43)nc3ccc4ccccc4c23)cc1]

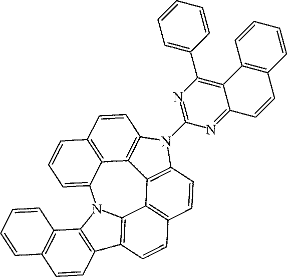 [SMILES: c1ccc(-c2nc(-n3c4ccc5cccc6c5c4c4c5c(ccc7c8ccc9ccccc9c8n6c75)ccc43)nc3ccc4ccccc4c23)cc1]

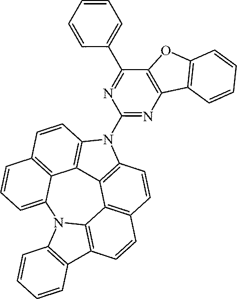 [SMILES: c1ccc(-c2nc(-n3c4ccc5cccc6c5c4c4c5c(ccc7c8ccccc8n6c75)ccc43)nc3c2oc2ccccc23)cc1]

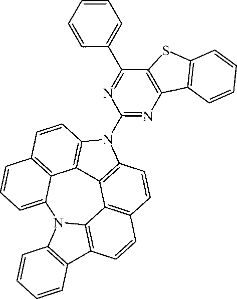 [SMILES: c1ccc(-c2nc(-n3c4ccc5cccc6c5c4c4c5c(ccc7c8ccccc8n6c75)ccc43)nc3c2sc2ccccc23)cc1]

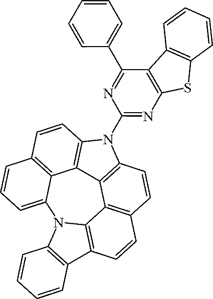 [SMILES: c1ccc(-c2nc(-n3c4ccc5cccc6c5c4c4c5c(ccc7c8ccccc8n6c75)ccc43)nc3sc4ccccc4c23)cc1]

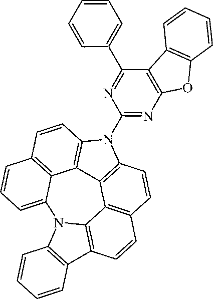 [SMILES: c1ccc(-c2nc(-n3c4ccc5cccc6c5c4c4c5c(ccc7c8ccccc8n6c75)ccc43)nc3oc4ccccc4c23)cc1]

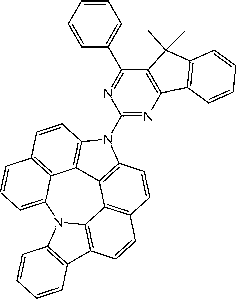 [SMILES: CC1(C)c2ccccc2-c2nc(-n3c4ccc5cccc6c5c4c4c5c(ccc7c8ccccc8n6c75)ccc43)nc(-c3ccccc3)c21]

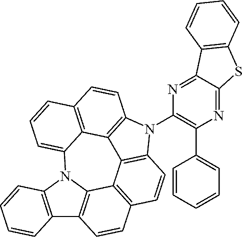 [SMILES: c1ccc(-c2nc3sc4ccccc4c3nc2-n2c3ccc4cccc5c4c3c3c4c(ccc6c7ccccc7n5c64)ccc32)cc1]

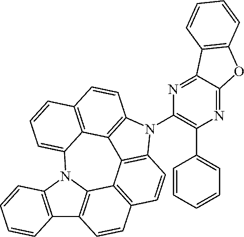 [SMILES: c1ccc(-c2nc3oc4ccccc4c3nc2-n2c3ccc4cccc5c4c3c3c4c(ccc6c7ccccc7n5c64)ccc32)cc1]

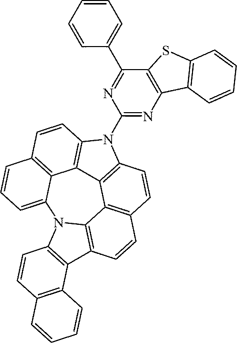 [SMILES: c1ccc(-c2nc(-n3c4ccc5cccc6c5c4c4c5c(ccc7c8c9ccccc9ccc8n6c75)ccc43)nc3c2sc2ccccc23)cc1]

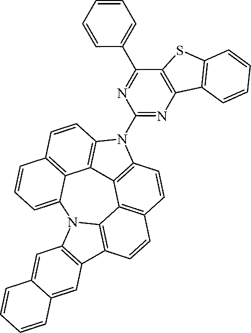 [SMILES: c1ccc(-c2nc(-n3c4ccc5cccc6c5c4c4c5c(ccc7c8cc9ccccc9cc8n6c75)ccc43)nc3c2sc2ccccc23)cc1]

In some embodiments, the glass transition temperature (Tg) of the organic compound as described herein ≥100° C. In some embodiments, Tg ≥120° C. In some embodiments, Tg ≥140° C. In some embodiments, Tg ≥160° C. In some embodiments, Tg ≥180° C.

The organic compound as described herein can be used as a functional material in electronic devices, especially OLED devices.

In some embodiments the compound as described herein can be used as a phosphorescent host material or a co-host material.

As the phosphorescent host material, the organic compound should have an appropriate triplet state energy level, namely T1. In some embodiments, the compound as described herein, its T1 is ≥ 1.9 eV, preferably ≥ 2.0 eV, more preferably ≥ 2.1 eV, and most preferably ≥ 2.2 eV.

In some circumstances, a smaller ΔE_{ST} of the phosphorescent main material is beneficial for the performance of the device. In some embodiments, the compound as described herein, its ΔE_{ST}≤0.35 eV, preferably ≤0.3 eV, more preferably ≤0.2 eV, and most preferably ≤0.15 eV.

In some embodiments, the compound as described herein, its ((HOMO-(HOMO-1))≥0.1 eV, preferably≥0.15 eV, more preferably≥0.2 eV, most preferably≥0.3 eV.

In some embodiments, the compound as described herein, its (((LUMO+1)-LUMO)≥0.15 eV, preferably≥0.25 eV, more preferably≥0.30 eV, most preferably≥0.35 eV.

In some embodiments, the organic compound as described herein is partially deuterated; preferably 10% or more of total H, more preferably 20% or more of total H, further preferably 30% or more of total H, and most preferably 40% or more of total H, are deuterated.

In some embodiments, the organic compound as described herein is a small molecular material.

In some embodiments, the organic compound as described herein is used for evaporation-based OLEDs. For this purpose, the molecular weight of the organic compound as described herein ≤1000 g/mol, preferably ≤900 g/mol, more preferably ≤850 g/mol, further preferably ≤800 g/mol, and most preferably ≤ 700 g/mol.

In another aspect, the present disclosure also provides a synthetic method of the organic compound according to formula (I), where feedstocks containing active groups are used to carry out the reaction. These active feedstocks comprise at least one leaving group, such as, a bromine, an iodine, a boronic acid, or boronic ester. The appropriate reactions for forming C—C linkages are familiar to the person skilled in the art and are described in the literature, particularly appropriate and preferred coupling reactions are the SUZUKI STILLE, and HECK.

In yet another aspect, the present disclosure further provides a polymer comprising at least one repeating unit, where at least one of the repeating unit comprises a structure of formula (I). In some embodiments, the polymer is a non-conjugated polymer in which the structural unit of formula (I) is on a side chain. In some embodiments, the polymer is a conjugated polymer. The term “small molecule” herein refers to a molecule that is no one of following: a polymer, an oligomer, a dendrimer, or a blend. In particular, there are no repeating structures in the small molecule. The molecular weight of the small molecule ≤3000 g/mol, preferably ≤2000 g/mol, and most preferably ≤1500 g/mol.

The term of polymer comprises homopolymer, copolymer, and block copolymer. Also in the present disclosure, the term of polymer comprises dendrimer. For the synthesis and application of the dendrimers, please refer to [Dendrimers and Dendrons, Wiley-VCH Verlag GmbH & Co. KGaA, 2002, Ed. George R. Newkome, Charles N. Moorefield, Fritz Vogtle.].

The term “conjugated polymer” refers to a polymer with backbone mainly comprising sp2 hybrid orbitals of C atoms, well-known examples are polyacetylene and poly(phenylene vinylene). The C atoms on the backbones can also be substituted with other non-C atoms. Moreover, the above-mentioned structure should still be considered as a conjugated polymer when the sp2 hybridization on the backbone is interrupted by natural defects. Also in the present disclosure, the backbone of the conjugated polymer comprises aryl amines, aryl phosphines and other heteroarmotics, organometallic complexes, etc.

In some embodiments, the synthetic method of the polymer is selected from the group consisting of SUZUKI-, YAMAMOTO-, STILLE-, NIGESHI-, KUMADA-, HECK-, SONOGASHIRA-, HIYAMA-, FUKUYAMA-, HARTWIG-, BUCHWALD-, and ULLMAN-.

In some embodiments, the glass transition temperature (Tg) of the polymer as described herein ≥ 100° C., preferably ≥120° C., more preferably ≥140° C., further preferably ≥160° C., and most preferably >180° C.

In some embodiments, in the polymer as described herein, the polydipersity index (PDI) is preferably from 1 to 5, more preferably 1 to 4; even more preferably 1 to 3, further preferably 1 to 2, and most preferably 1 to 1.5.

In some embodiments, in the polymer as described herein, the value range of the weight-average molecular weight (M_{w}) is preferably from 10k to 1 million, more preferably 50k to 500k, even more preferably 100k to 400k, further preferably 150k to 300k, and most preferably 200k to 250k.

In yet another aspect, the present disclosure further provides a mixture comprising an organic compound H1 and an organic compound H2, the organic compound H1 is selected from an organic compound as described herein, the organic compound H2 is another organic functional material, and is selected from a hole-injection material (HIM), a hole-transport material (HTM), a p-dopant material, an electron-transport material (ETM), an electron-injection material (EIM), an electron-blocking material (EBM), a hole-blocking material (HBM), an emitting material (Emitter), a host material (Host), or an organic dye. These organic functional materials are described in detail, for example, in US2017092880A1, US2018006247A1, and EP3301097A2. The entire contents of these three documents are incorporated herein by reference in their entirety.

In some embodiments, the mixture comprises at least one organic compound or polymer as described herein, and a hole-transport material (HTM).

In some embodiments, the mixture comprises at least one organic compound or polymer as described herein, and an emitting material, the emitting material is selected from the group consisting of a singlet emitting material (fluorescent emitting material), a triplet emitting material (phosphorescent emitting material), and a TADF material.

In some embodiments, the mixture comprises at least one organic compound or polymer as described herein, and a fluorescent emitter, where the weight percentage of the fluorescent emitter ≤10 wt%, preferably ≤9 wt%, more preferably ≤8 wt%, further preferably ≤7 wt%, and most preferably ≤5 wt%.

In some embodiments, the mixture comprises at least one organic compound or polymer as described herein, and a phosphorescent emitter, where the weight percentage of the phosphorescent emitter ≤20 wt%, preferably ≤15 wt%, and more preferably ≤10 wt%.

In some embodiments, the mixture comprises at least one organic compound or polymer as described herein, and a TADF material; where the property of the TADF material is preferably: 1) forming a co-host material with the organic compound as described herein in a weight ratio from 1:6 to 6:1; 2) forming an Exciplex with the organic compounds as described herein, or as an auxiliary light emitter, where the weight percentage of the TADF material ≤15 wt%, preferably ≤10 wt%, more preferably ≤8 wt%.

In some embodiments, the mixture comprises at least one organic compound or polymer as described herein, and an organic compound H2 (i. e. another organic functional material). Such a mixture can be used as a phosphorescent mixed host material, and can further comprise a phosphorescent emitter, where the weight percentage of the phosphorescent emitter ≤20 wt%, preferably ≤15 wt%, more preferably ≤10 wt%.

A detailed description of the mixture containing an organic compound H1 and an organic compound H2 as a phosphorescent hybrid host is given below.

In some embodiments, the organic compound H2 has hole-transport property.

More preferably, the organic compound H2 has both hole-transport property and electron-transport property.

Generally, the molar ratio of the organic compound H1 to the organic compound H2 ranges from 1:9 to 9:1.

Preferably, the molar ratio of the organic compound H1 to the organic compound H2 ranges from 3:7 to 7:3.

More preferably, the molar ratio of the organic compound H1 to the organic compound H2 ranges from 4:6 to 6:4.

Most preferably, the molar ratio of the organic compound H1 to the organic compound H2 is 5:5.

In some embodiments, the organic compound H2 of the mixture comprises a structure of formula (II):

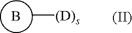

Where B is selected from a C₁-C₃₀ substituted/unsubstituted alkyl group, a C₃-C₃₀ substituted/unsubstituted cycloalkyl group, or a C₅-C₆₀ substituted/unsubstituted aromatic hydrocarbon or aromatic heterocyclic group; D is an electron-rich group; s is an integer from 1 to 6.

In some embodiments, the electron-rich (or electron donor) D group of formula (II) comprises any one of the following groups or any combination thereof:

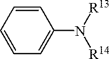

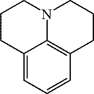

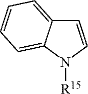

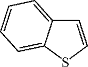

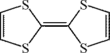

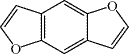

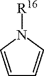

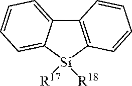

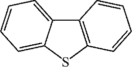

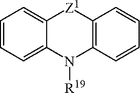

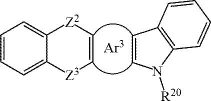

Where Ar³ represents a C₅-C₄₀ aromatic or heteroaromatic group; Z¹, Z², Z³ are independently selected from a single bond, CR²¹R²², SiR²³R²⁴, NR²⁵, O, C(═O), S, S═O, or SO₂, and Z² and Z³ are not single bonds at the same time; R¹³ to R²⁵ are identically defined as the above-mentioned R₁.

In some embodiments, the electron-rich (or electron donor) D group of formula (II) comprises any one of the following groups:

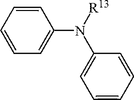

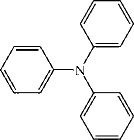 [SMILES: c1ccc(N(c2ccccc2)c2ccccc2)cc1]

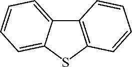 [SMILES: c1ccc2c(c1)sc1ccccc12]

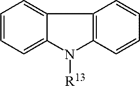

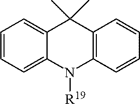

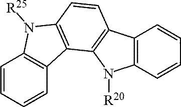

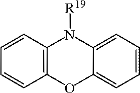

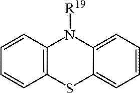

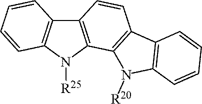

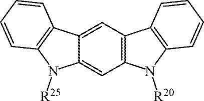

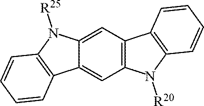

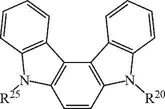

R¹³, R¹⁹, R²⁰, and R²⁵ are identically defined as described above.

In some embodiments, s is an integer from 1 to 4. In some embodiments, s is an integer from 1 to 3. In some embodiments, s is 1 or 2.

In some embodiments, the organic compound H2 of the mixture is selected from one of the following structural formulas:

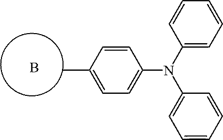

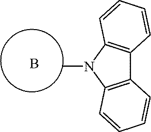

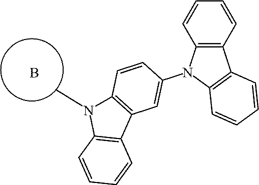

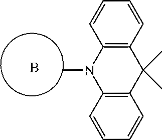

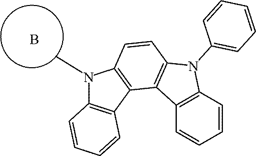

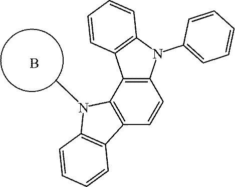

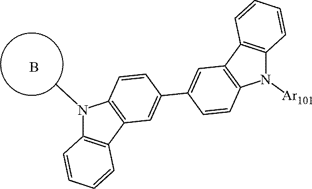

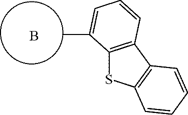

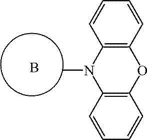

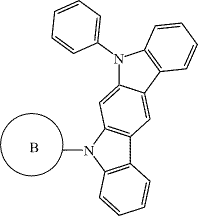

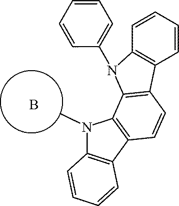

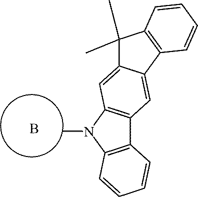

Where each B is defined as described above, and Ar₁₀₁ is defined as Ar₁.

In some embodiments, in the mixture as described herein, the organic compound H1 and/or the organic compound H2 have a high triplet energy level E_{T1}, generally E_{T1} ≥2.0 eV, more preferably E_{T1}≥2.1 eV, even more preferably E_{T1}≥2.2 eV, further preferably E_{T1}≥2.4 eV, and most preferably E_{T1}≥2.6 eV.

In some embodiments, the organic compound H1 and the organic compound H2 of the mixture form a type II heterojunction energy structure, i. e., the highest occupied orbital level (HOMO) of the organic compound H1 is lower than the HOMO of the organic compound H2, and the lowest unoccupied orbital level (LUMO) of the organic compound H1 is lower than the LUMO of the organic compound H2.

In some embodiments, in the mixture as described herein, min (LUMO(H1)-HOMO(H2), LUMO(H2)-HOMO(H1)) ≤min(ET(H1), ET(H2))+0.1 eV, where LUMO(H1), HOMO(H1), and ET(H1) are the lowest unoccupied orbital, the highest occupied orbital, and the triplet energy level of the organic compound H1, respectively. LUMO(H2), HOMO(H2) and ET(H2) are the lowest unoccupied orbital, the highest occupied orbital, and the triplet energy level of the organic compound H2, respectively.

In some embodiments, in the mixture as described herein, min(LUMO(H1)-HOMO(H2), LUMO(H2)-HOMO(H1)) ≤min(ET(H1), ET(H2)).

In some embodiments, in the mixture as described herein, min(LUMO(H1)-HOMO(H2), LUMO(H2)-HOMO(H1)) ≤min(ET(H1), ET(H2))-0.05 eV.

In some embodiments, in the mixture as described herein, min (LUMO(H1)-HOMO(H2), LUMO(H2)-HOMO(H1)) ≤min(ET(H1), ET(H2))-0.1 eV.

In some embodiments, in the mixture as described herein, min(LUMO(H1)-HOMO(H2), LUMO(H2)-HOMO(H1)) ≤min(ET(H1), ET(H2))-0.15 eV.

In some embodiments, in the mixture as described herein, min(LUMO(H1)-HOMO(H2), LUMO(H2)-HOMO(H1)) ≤min(ET(H1), ET(H2))-0.2 eV.

Specific examples of the organic compound H2 according to formula (II) are shown below, but not limited thereto:

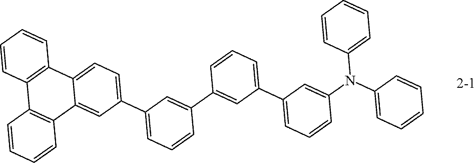 [SMILES: c1ccc(N(c2ccccc2)c2cccc(-c3cccc(-c4cccc(-c5ccc6c7ccccc7c7ccccc7c6c5)c4)c3)c2)cc1]

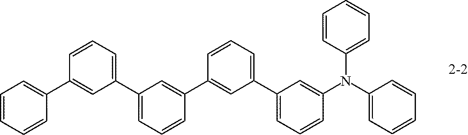 [SMILES: c1ccc(-c2cccc(-c3cccc(-c4cccc(-c5cccc(N(c6ccccc6)c6ccccc6)c5)c4)c3)c2)cc1]

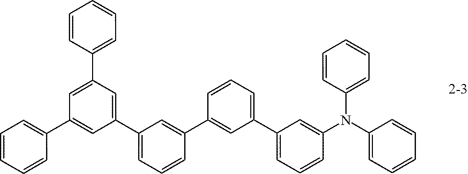 [SMILES: c1ccc(-c2cc(-c3ccccc3)cc(-c3cccc(-c4cccc(-c5cccc(N(c6ccccc6)c6ccccc6)c5)c4)c3)c2)cc1]

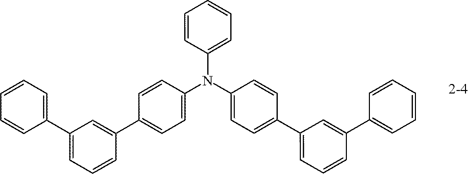 [SMILES: c1ccc(-c2cccc(-c3ccc(N(c4ccccc4)c4ccc(-c5cccc(-c6ccccc6)c5)cc4)cc3)c2)cc1]

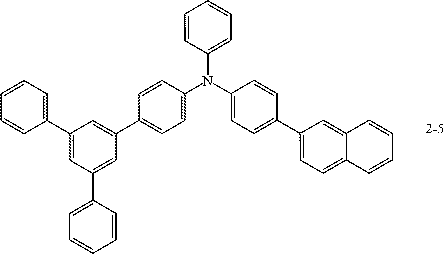 [SMILES: c1ccc(-c2cc(-c3ccccc3)cc(-c3ccc(N(c4ccccc4)c4ccc(-c5ccc6ccccc6c5)cc4)cc3)c2)cc1]

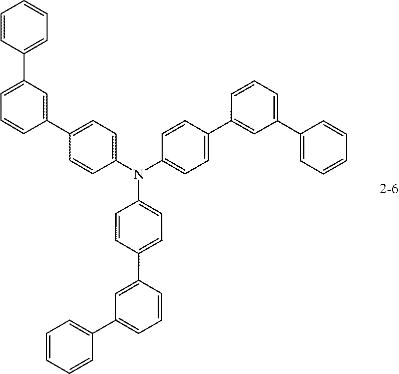 [SMILES: c1ccc(-c2cccc(-c3ccc(N(c4ccc(-c5cccc(-c6ccccc6)c5)cc4)c4ccc(-c5cccc(-c6ccccc6)c5)cc4)cc3)c2)cc1]

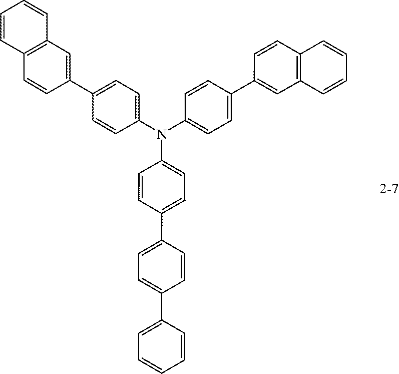 [SMILES: c1ccc(-c2ccc(-c3ccc(N(c4ccc(-c5ccc6ccccc6c5)cc4)c4ccc(-c5ccc6ccccc6c5)cc4)cc3)cc2)cc1]

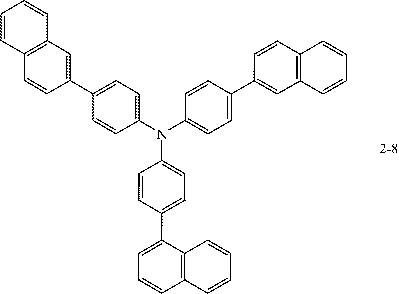 [SMILES: c1ccc2cc(-c3ccc(N(c4ccc(-c5ccc6ccccc6c5)cc4)c4ccc(-c5cccc6ccccc56)cc4)cc3)ccc2c1]

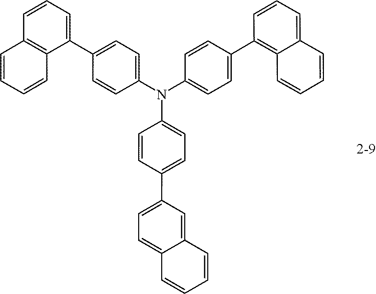 [SMILES: c1ccc2cc(-c3ccc(N(c4ccc(-c5cccc6ccccc56)cc4)c4ccc(-c5cccc6ccccc56)cc4)cc3)ccc2c1]

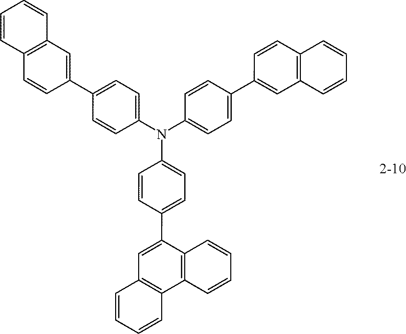 [SMILES: c1ccc2cc(-c3ccc(N(c4ccc(-c5ccc6ccccc6c5)cc4)c4ccc(-c5cc6ccccc6c6ccccc56)cc4)cc3)ccc2c1]

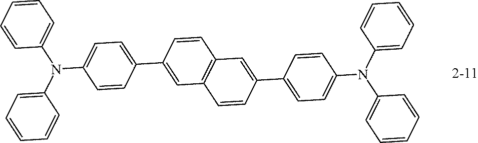 [SMILES: c1ccc(N(c2ccccc2)c2ccc(-c3ccc4cc(-c5ccc(N(c6ccccc6)c6ccccc6)cc5)ccc4c3)cc2)cc1]

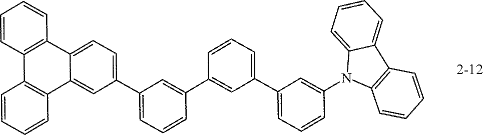 [SMILES: c1cc(-c2cccc(-c3ccc4c5ccccc5c5ccccc5c4c3)c2)cc(-c2cccc(-n3c4ccccc4c4ccccc43)c2)c1]

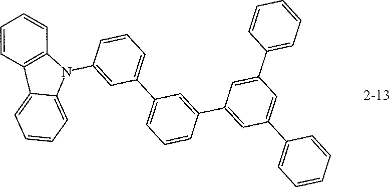 [SMILES: c1ccc(-c2cc(-c3ccccc3)cc(-c3cccc(-c4cccc(-n5c6ccccc6c6ccccc65)c4)c3)c2)cc1]

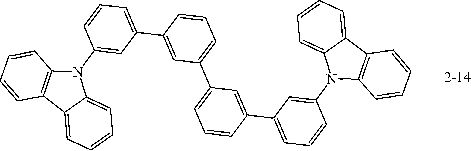 [SMILES: c1cc(-c2cccc(-c3cccc(-n4c5ccccc5c5ccccc54)c3)c2)cc(-c2cccc(-n3c4ccccc4c4ccccc43)c2)c1]

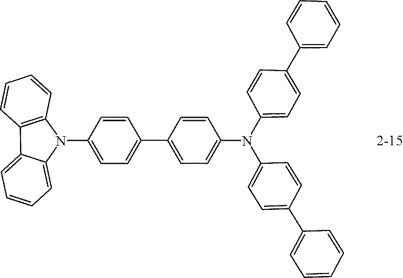 [SMILES: c1ccc(-c2ccc(N(c3ccc(-c4ccccc4)cc3)c3ccc(-c4ccc(-n5c6ccccc6c6ccccc65)cc4)cc3)cc2)cc1]

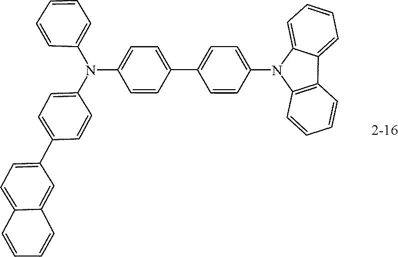 [SMILES: c1ccc(N(c2ccc(-c3ccc(-n4c5ccccc5c5ccccc54)cc3)cc2)c2ccc(-c3ccc4ccccc4c3)cc2)cc1]

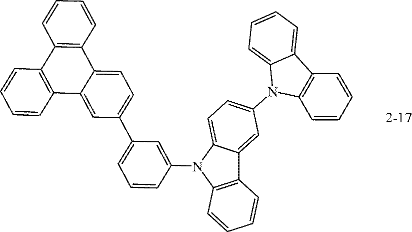 [SMILES: c1cc(-c2ccc3c4ccccc4c4ccccc4c3c2)cc(-n2c3ccccc3c3cc(-n4c5ccccc5c5ccccc54)ccc32)c1]

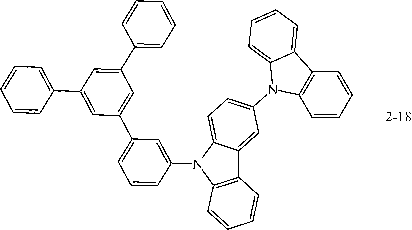 [SMILES: c1ccc(-c2cc(-c3ccccc3)cc(-c3cccc(-n4c5ccccc5c5cc(-n6c7ccccc7c7ccccc76)ccc54)c3)c2)cc1]

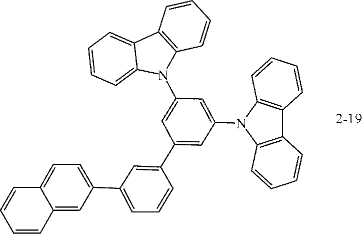 [SMILES: c1cc(-c2cc(-n3c4ccccc4c4ccccc43)cc(-n3c4ccccc4c4ccccc43)c2)cc(-c2ccc3ccccc3c2)c1]

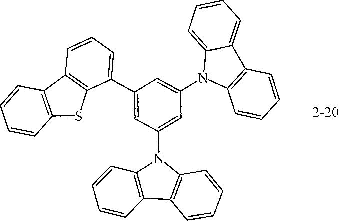 [SMILES: c1ccc2c(c1)sc1c(-c3cc(-n4c5ccccc5c5ccccc54)cc(-n4c5ccccc5c5ccccc54)c3)cccc12]

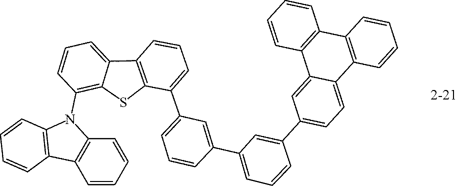 [SMILES: c1cc(-c2cccc(-c3cccc4c3sc3c(-n5c6ccccc6c6ccccc65)cccc34)c2)cc(-c2ccc3c4ccccc4c4ccccc4c3c2)c1]

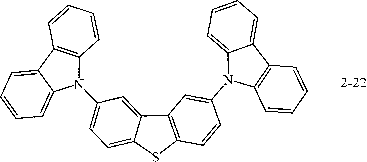 [SMILES: c1ccc2c(c1)c1ccccc1n2-c1ccc2sc3ccc(-n4c5ccccc5c5ccccc54)cc3c2c1]

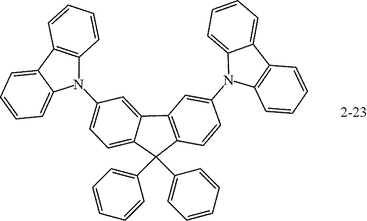 [SMILES: c1ccc(C2(c3ccccc3)c3ccc(-n4c5ccccc5c5ccccc54)cc3-c3cc(-n4c5ccccc5c5ccccc54)ccc32)cc1]

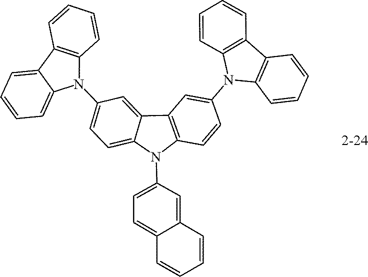 [SMILES: c1ccc2cc(-n3c4ccc(-n5c6ccccc6c6ccccc65)cc4c4cc(-n5c6ccccc6c6ccccc65)ccc43)ccc2c1]

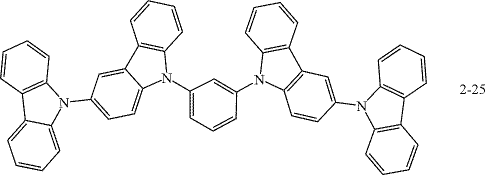 [SMILES: c1cc(-n2c3ccccc3c3cc(-n4c5ccccc5c5ccccc54)ccc32)cc(-n2c3ccccc3c3cc(-n4c5ccccc5c5ccccc54)ccc32)c1]

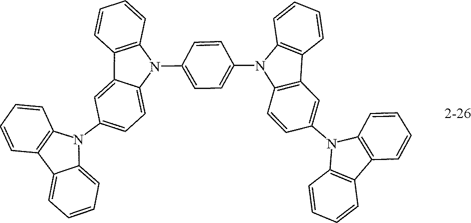 [SMILES: c1ccc2c(c1)c1ccccc1n2-c1ccc2c(c1)c1ccccc1n2-c1ccc(-n2c3ccccc3c3cc(-n4c5ccccc5c5ccccc54)ccc32)cc1]

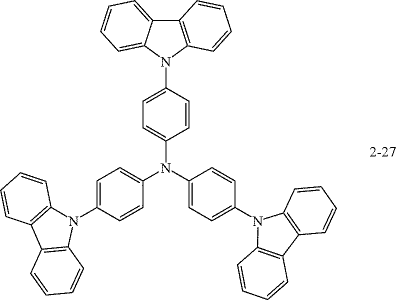 [SMILES: c1ccc2c(c1)c1ccccc1n2-c1ccc(N(c2ccc(-n3c4ccccc4c4ccccc43)cc2)c2ccc(-n3c4ccccc4c4ccccc43)cc2)cc1]

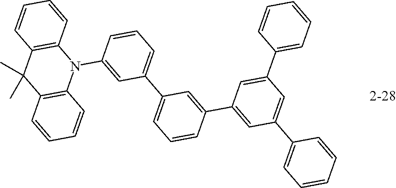 [SMILES: CC1(C)c2ccccc2N(c2cccc(-c3cccc(-c4cc(-c5ccccc5)cc(-c5ccccc5)c4)c3)c2)c2ccccc21]

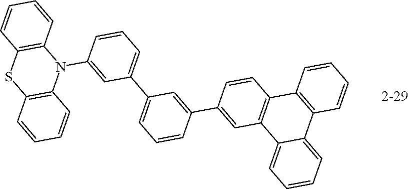 [SMILES: c1cc(-c2cccc(N3c4ccccc4Sc4ccccc43)c2)cc(-c2ccc3c4ccccc4c4ccccc4c3c2)c1]

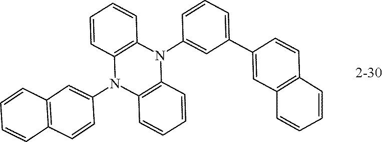 [SMILES: c1cc(-c2ccc3ccccc3c2)cc(N2c3ccccc3N(c3ccc4ccccc4c3)c3ccccc32)c1]

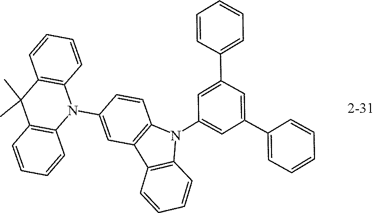 [SMILES: CC1(C)c2ccccc2N(c2ccc3c(c2)c2ccccc2n3-c2cc(-c3ccccc3)cc(-c3ccccc3)c2)c2ccccc21]

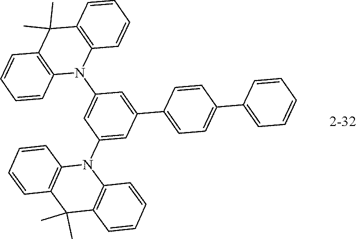 [SMILES: CC1(C)c2ccccc2N(c2cc(-c3ccc(-c4ccccc4)cc3)cc(N3c4ccccc4C(C)(C)c4ccccc43)c2)c2ccccc21]

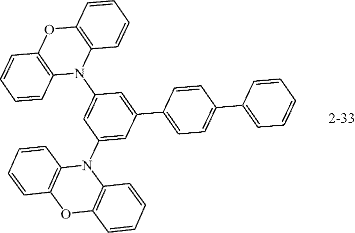 [SMILES: c1ccc(-c2ccc(-c3cc(N4c5ccccc5Oc5ccccc54)cc(N4c5ccccc5Oc5ccccc54)c3)cc2)cc1]

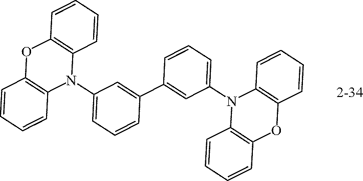 [SMILES: c1cc(-c2cccc(N3c4ccccc4Oc4ccccc43)c2)cc(N2c3ccccc3Oc3ccccc32)c1]

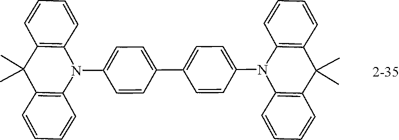

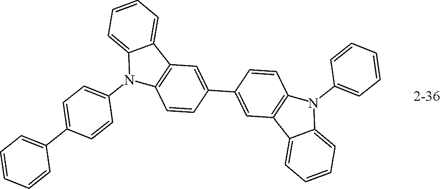 [SMILES: c1ccc(-c2ccc(-n3c4ccccc4c4cc(-c5ccc6c(c5)c5ccccc5n6-c5ccccc5)ccc43)cc2)cc1]

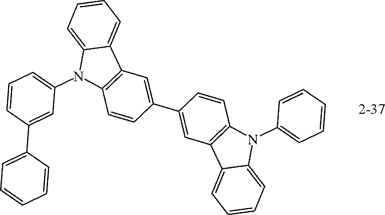 [SMILES: c1ccc(-c2cccc(-n3c4ccccc4c4cc(-c5ccc6c(c5)c5ccccc5n6-c5ccccc5)ccc43)c2)cc1]

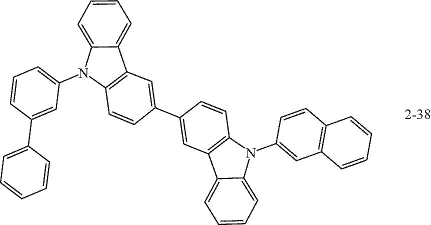 [SMILES: c1ccc(-c2cccc(-n3c4ccccc4c4cc(-c5ccc6c(c5)c5ccccc5n6-c5ccc6ccccc6c5)ccc43)c2)cc1]

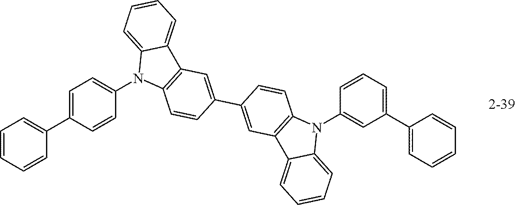 [SMILES: c1ccc(-c2ccc(-n3c4ccccc4c4cc(-c5ccc6c(c5)c5ccccc5n6-c5cccc(-c6ccccc6)c5)ccc43)cc2)cc1]

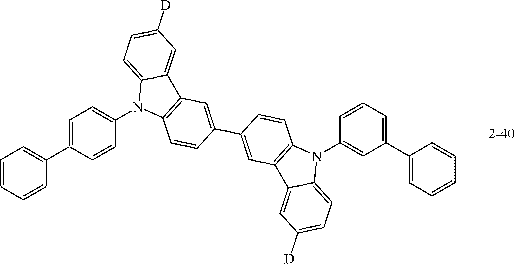 [SMILES: [2H]c1ccc2c(c1)c1cc(-c3ccc4c(c3)c3cc([2H])ccc3n4-c3cccc(-c4ccccc4)c3)ccc1n2-c1ccc(-c2ccccc2)cc1]

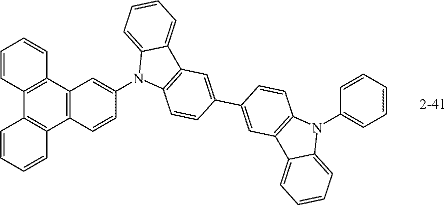 [SMILES: c1ccc(-n2c3ccccc3c3cc(-c4ccc5c(c4)c4ccccc4n5-c4ccc5c6ccccc6c6ccccc6c5c4)ccc32)cc1]

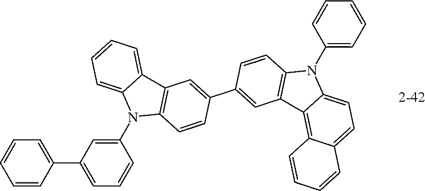 [SMILES: c1ccc(-c2cccc(-n3c4ccccc4c4cc(-c5ccc6c(c5)c5c7ccccc7ccc5n6-c5ccccc5)ccc43)c2)cc1]

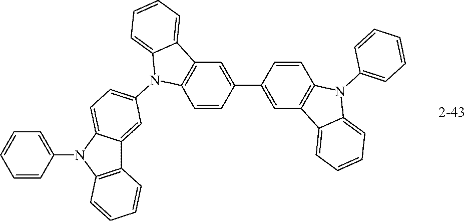 [SMILES: c1ccc(-n2c3ccccc3c3cc(-c4ccc5c(c4)c4ccccc4n5-c4ccc5c(c4)c4ccccc4n5-c4ccccc4)ccc32)cc1]

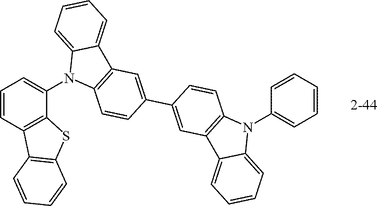 [SMILES: c1ccc(-n2c3ccccc3c3cc(-c4ccc5c(c4)c4ccccc4n5-c4cccc5c4sc4ccccc45)ccc32)cc1]

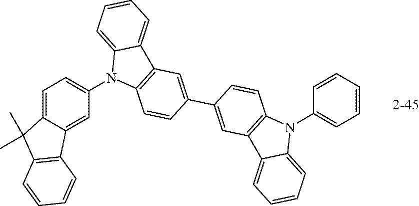 [SMILES: CC1(C)c2ccccc2-c2cc(-n3c4ccccc4c4cc(-c5ccc6c(c5)c5ccccc5n6-c5ccccc5)ccc43)ccc21]

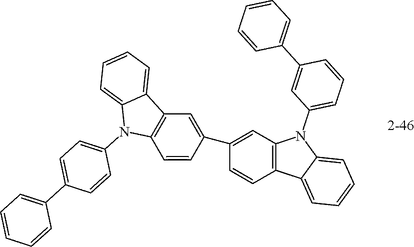 [SMILES: c1ccc(-c2ccc(-n3c4ccccc4c4cc(-c5ccc6c7ccccc7n(-c7cccc(-c8ccccc8)c7)c6c5)ccc43)cc2)cc1]

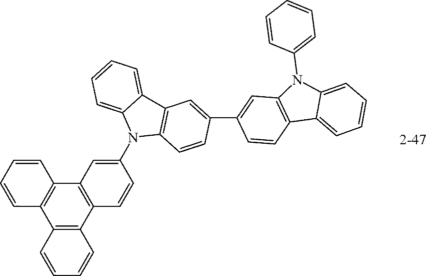 [SMILES: c1ccc(-n2c3ccccc3c3ccc(-c4ccc5c(c4)c4ccccc4n5-c4ccc5c6ccccc6c6ccccc6c5c4)cc32)cc1]

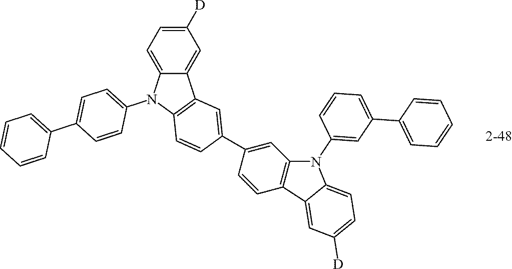 [SMILES: [2H]c1ccc2c(c1)c1cc(-c3ccc4c5cc([2H])ccc5n(-c5cccc(-c6ccccc6)c5)c4c3)ccc1n2-c1ccc(-c2ccccc2)cc1]

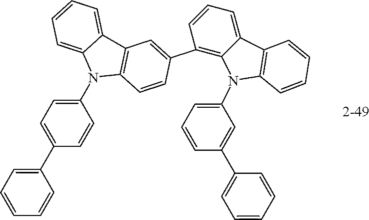 [SMILES: c1ccc(-c2ccc(-n3c4ccccc4c4cc(-c5cccc6c7ccccc7n(-c7cccc(-c8ccccc8)c7)c56)ccc43)cc2)cc1]

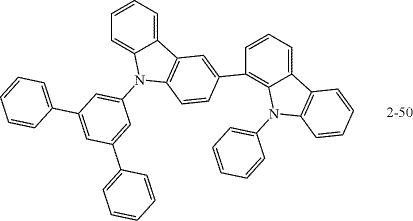 [SMILES: c1ccc(-c2cc(-c3ccccc3)cc(-n3c4ccccc4c4cc(-c5cccc6c7ccccc7n(-c7ccccc7)c56)ccc43)c2)cc1]

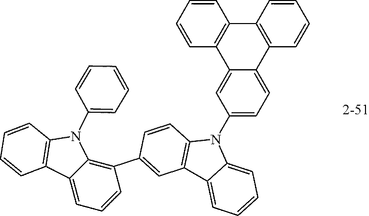 [SMILES: c1ccc(-n2c3ccccc3c3cccc(-c4ccc5c(c4)c4ccccc4n5-c4ccc5c6ccccc6c6ccccc6c5c4)c32)cc1]

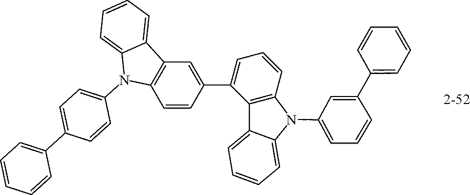 [SMILES: c1ccc(-c2ccc(-n3c4ccccc4c4cc(-c5cccc6c5c5ccccc5n6-c5cccc(-c6ccccc6)c5)ccc43)cc2)cc1]

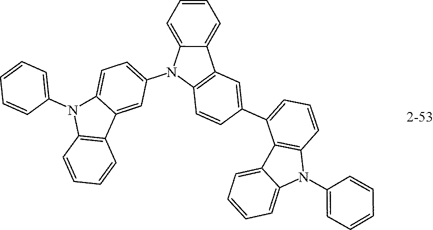 [SMILES: c1ccc(-n2c3ccccc3c3cc(-n4c5ccccc5c5cc(-c6cccc7c6c6ccccc6n7-c6ccccc6)ccc54)ccc32)cc1]

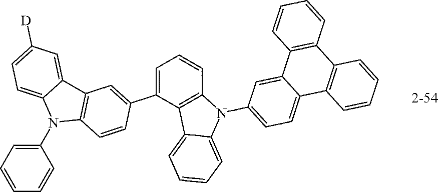 [SMILES: [2H]c1ccc2c(c1)c1cc(-c3cccc4c3c3ccccc3n4-c3ccc4c5ccccc5c5ccccc5c4c3)ccc1n2-c1ccccc1]

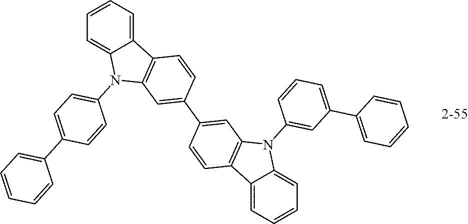 [SMILES: c1ccc(-c2ccc(-n3c4ccccc4c4ccc(-c5ccc6c7ccccc7n(-c7cccc(-c8ccccc8)c7)c6c5)cc43)cc2)cc1]

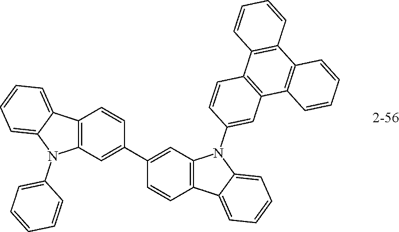 [SMILES: c1ccc(-n2c3ccccc3c3ccc(-c4ccc5c6ccccc6n(-c6ccc7c8ccccc8c8ccccc8c7c6)c5c4)cc32)cc1]

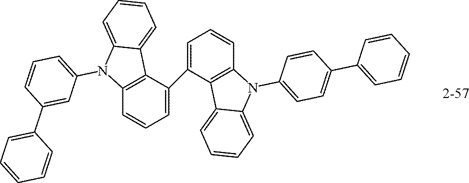 [SMILES: c1ccc(-c2ccc(-n3c4ccccc4c4c(-c5cccc6c5c5ccccc5n6-c5cccc(-c6ccccc6)c5)cccc43)cc2)cc1]

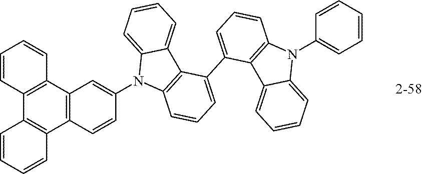 [SMILES: c1ccc(-n2c3ccccc3c3c(-c4cccc5c4c4ccccc4n5-c4ccc5c6ccccc6c6ccccc6c5c4)cccc32)cc1]

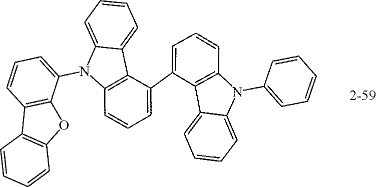 [SMILES: c1ccc(-n2c3ccccc3c3c(-c4cccc5c4c4ccccc4n5-c4cccc5c4oc4ccccc45)cccc32)cc1]

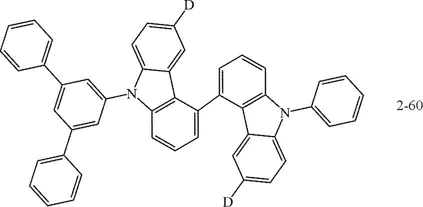 [SMILES: [2H]c1ccc2c(c1)c1c(-c3cccc4c3c3cc([2H])ccc3n4-c3cc(-c4ccccc4)cc(-c4ccccc4)c3)cccc1n2-c1ccccc1]

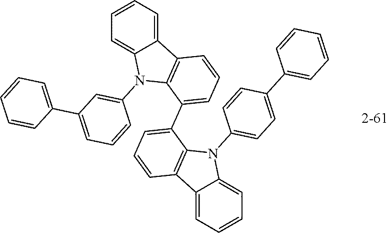 [SMILES: c1ccc(-c2ccc(-n3c4ccccc4c4cccc(-c5cccc6c7ccccc7n(-c7cccc(-c8ccccc8)c7)c56)c43)cc2)cc1]

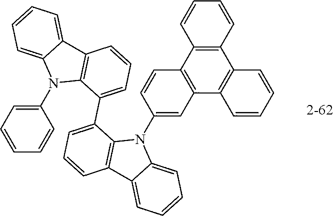 [SMILES: c1ccc(-n2c3ccccc3c3cccc(-c4cccc5c6ccccc6n(-c6ccc7c8ccccc8c8ccccc8c7c6)c45)c32)cc1]

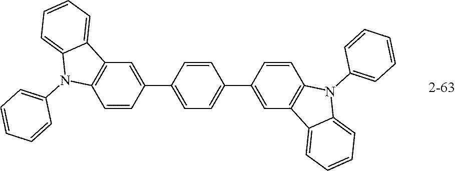 [SMILES: c1ccc(-n2c3ccccc3c3cc(-c4ccc(-c5ccc6c(c5)c5ccccc5n6-c5ccccc5)cc4)ccc32)cc1]

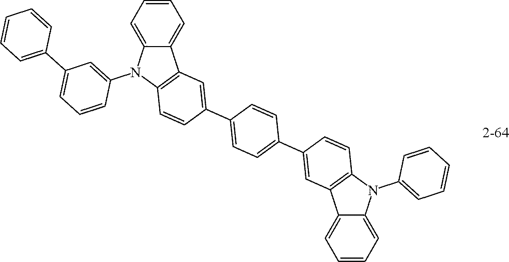 [SMILES: c1ccc(-c2cccc(-n3c4ccccc4c4cc(-c5ccc(-c6ccc7c(c6)c6ccccc6n7-c6ccccc6)cc5)ccc43)c2)cc1]

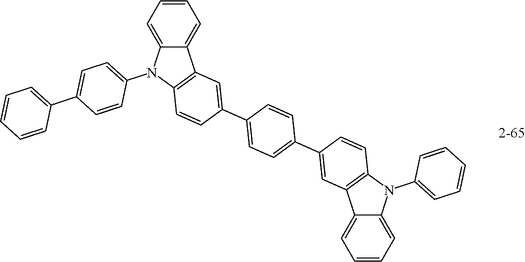 [SMILES: c1ccc(-c2ccc(-n3c4ccccc4c4cc(-c5ccc(-c6ccc7c(c6)c6ccccc6n7-c6ccccc6)cc5)ccc43)cc2)cc1]

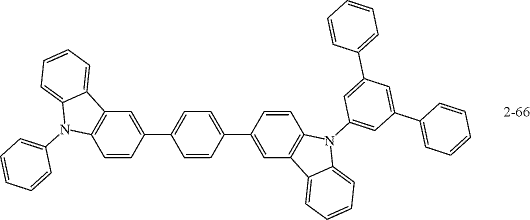 [SMILES: c1ccc(-c2cc(-c3ccccc3)cc(-n3c4ccccc4c4cc(-c5ccc(-c6ccc7c(c6)c6ccccc6n7-c6ccccc6)cc5)ccc43)c2)cc1]

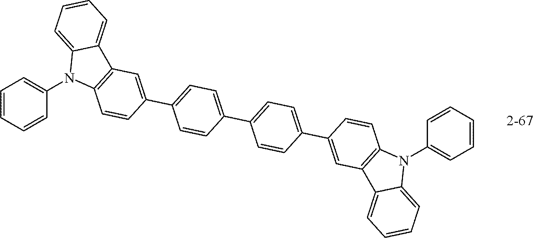 [SMILES: c1ccc(-n2c3ccccc3c3cc(-c4ccc(-c5ccc(-c6ccc7c(c6)c6ccccc6n7-c6ccccc6)cc5)cc4)ccc32)cc1]

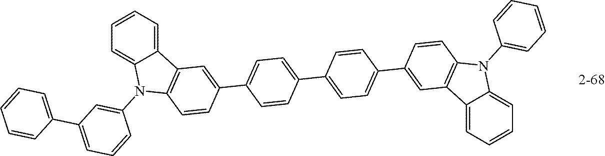 [SMILES: c1ccc(-c2cccc(-n3c4ccccc4c4cc(-c5ccc(-c6ccc(-c7ccc8c(c7)c7ccccc7n8-c7ccccc7)cc6)cc5)ccc43)c2)cc1]

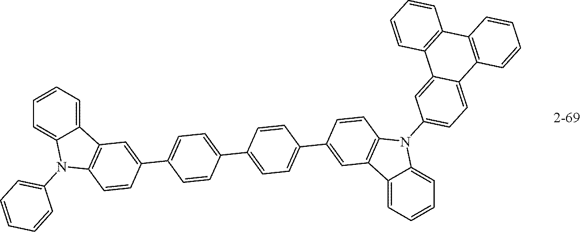 [SMILES: c1ccc(-n2c3ccccc3c3cc(-c4ccc(-c5ccc(-c6ccc7c(c6)c6ccccc6n7-c6ccc7c8ccccc8c8ccccc8c7c6)cc5)cc4)ccc32)cc1]

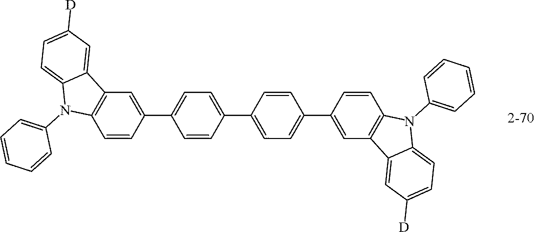 [SMILES: [2H]c1ccc2c(c1)c1cc(-c3ccc(-c4ccc(-c5ccc6c(c5)c5cc([2H])ccc5n6-c5ccccc5)cc4)cc3)ccc1n2-c1ccccc1]

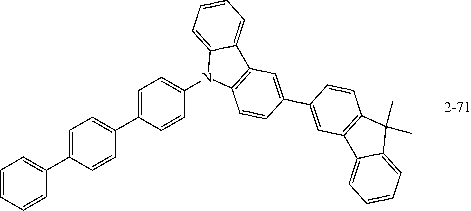 [SMILES: CC1(C)c2ccccc2-c2cc(-c3ccc4c(c3)c3ccccc3n4-c3ccc(-c4ccc(-c5ccccc5)cc4)cc3)ccc21]

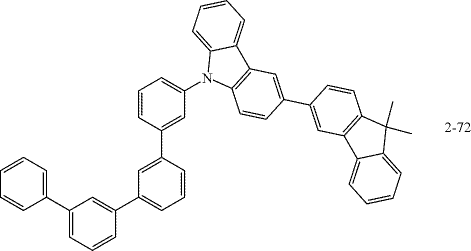 [SMILES: CC1(C)c2ccccc2-c2cc(-c3ccc4c(c3)c3ccccc3n4-c3cccc(-c4cccc(-c5cccc(-c6ccccc6)c5)c4)c3)ccc21]

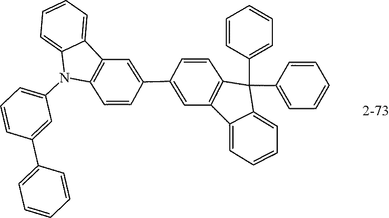 [SMILES: c1ccc(-c2cccc(-n3c4ccccc4c4cc(-c5ccc6c(c5)-c5ccccc5C6(c5ccccc5)c5ccccc5)ccc43)c2)cc1]

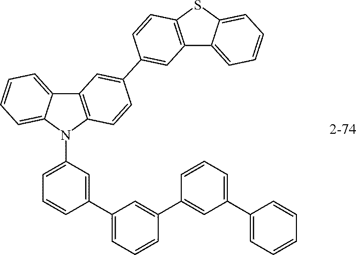 [SMILES: c1ccc(-c2cccc(-c3cccc(-c4cccc(-n5c6ccccc6c6cc(-c7ccc8sc9ccccc9c8c7)ccc65)c4)c3)c2)cc1]

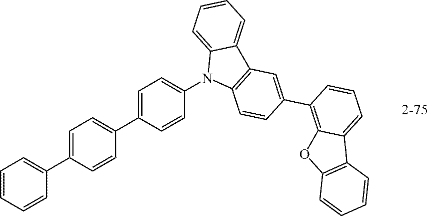 [SMILES: c1ccc(-c2ccc(-c3ccc(-n4c5ccccc5c5cc(-c6cccc7c6oc6ccccc67)ccc54)cc3)cc2)cc1]

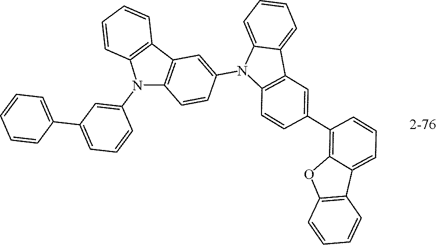 [SMILES: c1ccc(-c2cccc(-n3c4ccccc4c4cc(-n5c6ccccc6c6cc(-c7cccc8c7oc7ccccc78)ccc65)ccc43)c2)cc1]

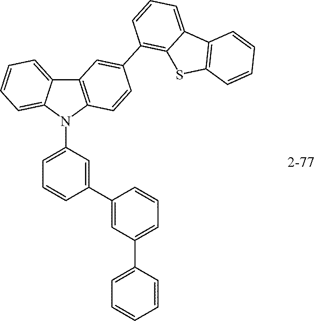 [SMILES: c1ccc(-c2cccc(-c3cccc(-n4c5ccccc5c5cc(-c6cccc7c6sc6ccccc67)ccc54)c3)c2)cc1]

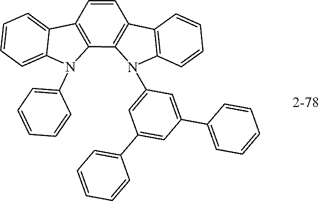 [SMILES: c1ccc(-c2cc(-c3ccccc3)cc(-n3c4ccccc4c4ccc5c6ccccc6n(-c6ccccc6)c5c43)c2)cc1]

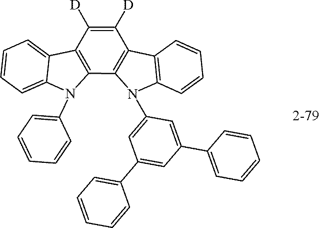 [SMILES: [2H]c1c([2H])c2c3ccccc3n(-c3cc(-c4ccccc4)cc(-c4ccccc4)c3)c2c2c1c1ccccc1n2-c1ccccc1]

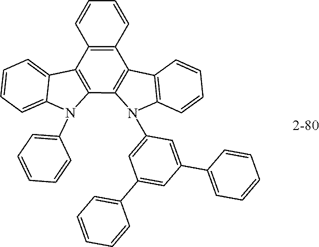 [SMILES: c1ccc(-c2cc(-c3ccccc3)cc(-n3c4ccccc4c4c5ccccc5c5c6ccccc6n(-c6ccccc6)c5c43)c2)cc1]

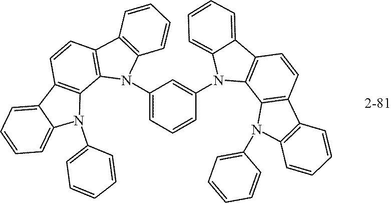 [SMILES: c1ccc(-n2c3ccccc3c3ccc4c5ccccc5n(-c5cccc(-n6c7ccccc7c7ccc8c9ccccc9n(-c9ccccc9)c8c76)c5)c4c32)cc1]

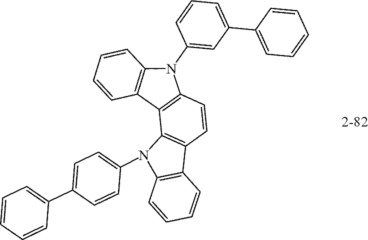 [SMILES: c1ccc(-c2ccc(-n3c4ccccc4c4ccc5c(c6ccccc6n5-c5cccc(-c6ccccc6)c5)c43)cc2)cc1]

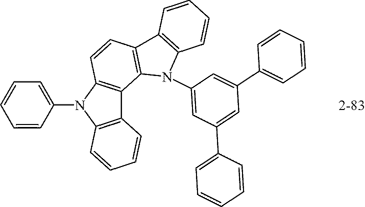 [SMILES: c1ccc(-c2cc(-c3ccccc3)cc(-n3c4ccccc4c4ccc5c(c6ccccc6n5-c5ccccc5)c43)c2)cc1]

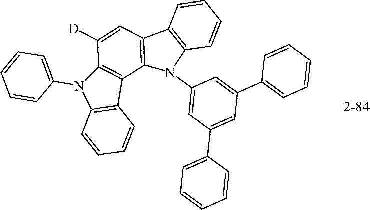 [SMILES: [2H]c1cc2c3ccccc3n(-c3cc(-c4ccccc4)cc(-c4ccccc4)c3)c2c2c3ccccc3n(-c3ccccc3)c12]

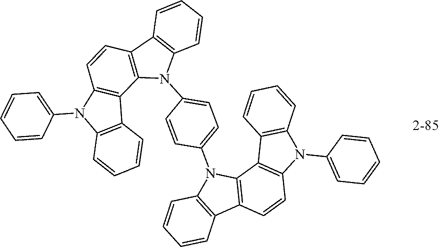 [SMILES: c1ccc(-n2c3ccccc3c3c2ccc2c4ccccc4n(-c4ccc(-n5c6ccccc6c6ccc7c(c8ccccc8n7-c7ccccc7)c65)cc4)c23)cc1]

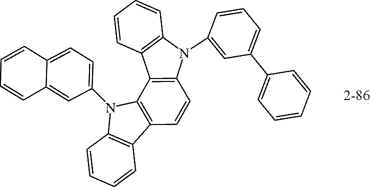 [SMILES: c1ccc(-c2cccc(-n3c4ccccc4c4c3ccc3c5ccccc5n(-c5ccc6ccccc6c5)c34)c2)cc1]

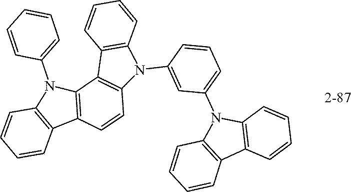 [SMILES: c1ccc(-n2c3ccccc3c3ccc4c(c5ccccc5n4-c4cccc(-n5c6ccccc6c6ccccc65)c4)c32)cc1]

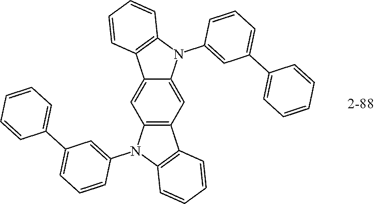 [SMILES: c1ccc(-c2cccc(-n3c4ccccc4c4cc5c(cc43)c3ccccc3n5-c3cccc(-c4ccccc4)c3)c2)cc1]

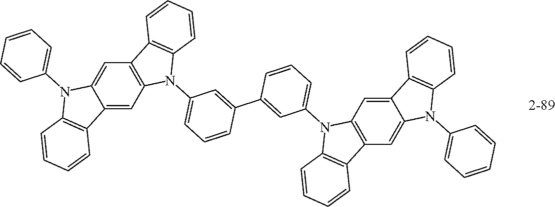 [SMILES: c1ccc(-n2c3ccccc3c3cc4c(cc32)c2ccccc2n4-c2cccc(-c3cccc(-n4c5ccccc5c5cc6c(cc54)c4ccccc4n6-c4ccccc4)c3)c2)cc1]

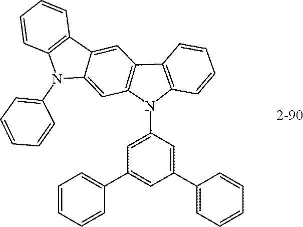 [SMILES: c1ccc(-c2cc(-c3ccccc3)cc(-n3c4ccccc4c4cc5c6ccccc6n(-c6ccccc6)c5cc43)c2)cc1]

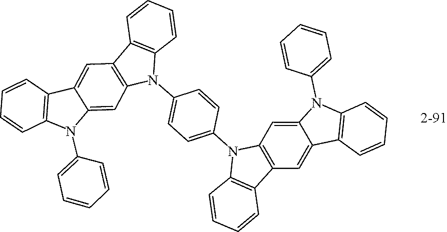 [SMILES: c1ccc(-n2c3ccccc3c3cc4c5ccccc5n(-c5ccc(-n6c7ccccc7c7cc8c9ccccc9n(-c9ccccc9)c8cc76)cc5)c4cc32)cc1]

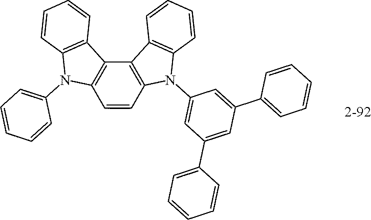 [SMILES: c1ccc(-c2cc(-c3ccccc3)cc(-n3c4ccccc4c4c5c6ccccc6n(-c6ccccc6)c5ccc43)c2)cc1]

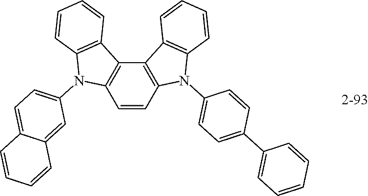 [SMILES: c1ccc(-c2ccc(-n3c4ccccc4c4c5c6ccccc6n(-c6ccc7ccccc7c6)c5ccc43)cc2)cc1]

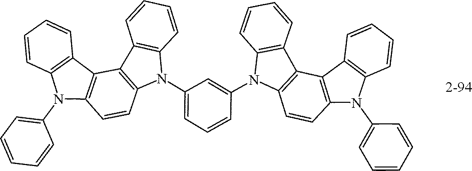 [SMILES: c1ccc(-n2c3ccccc3c3c4c5ccccc5n(-c5cccc(-n6c7ccccc7c7c8c9ccccc9n(-c9ccccc9)c8ccc76)c5)c4ccc32)cc1]

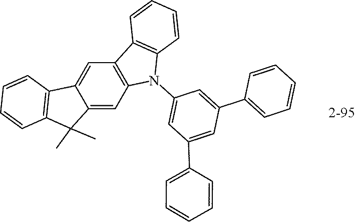 [SMILES: CC1(C)c2ccccc2-c2cc3c4ccccc4n(-c4cc(-c5ccccc5)cc(-c5ccccc5)c4)c3cc21]

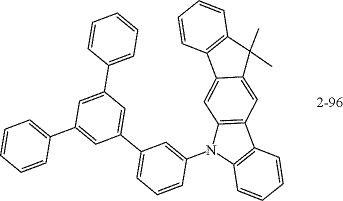 [SMILES: CC1(C)c2ccccc2-c2cc3c(cc21)c1ccccc1n3-c1cccc(-c2cc(-c3ccccc3)cc(-c3ccccc3)c2)c1]

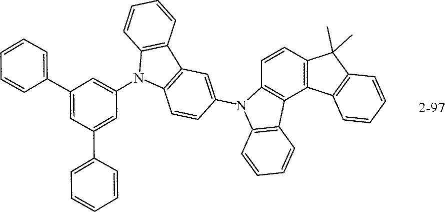 [SMILES: CC1(C)c2ccccc2-c2c1ccc1c2c2ccccc2n1-c1ccc2c(c1)c1ccccc1n2-c1cc(-c2ccccc2)cc(-c2ccccc2)c1]

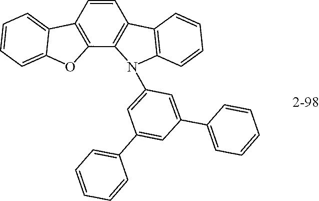 [SMILES: c1ccc(-c2cc(-c3ccccc3)cc(-n3c4ccccc4c4ccc5c6ccccc6oc5c43)c2)cc1]

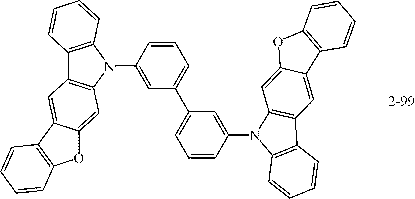 [SMILES: c1cc(-c2cccc(-n3c4ccccc4c4cc5c(cc43)oc3ccccc35)c2)cc(-n2c3ccccc3c3cc4c(cc32)oc2ccccc24)c1]

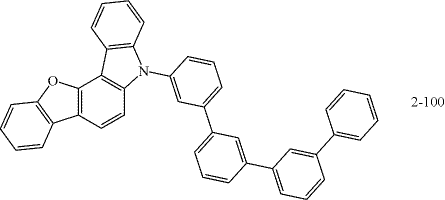 [SMILES: c1ccc(-c2cccc(-c3cccc(-c4cccc(-n5c6ccccc6c6c7oc8ccccc8c7ccc65)c4)c3)c2)cc1]

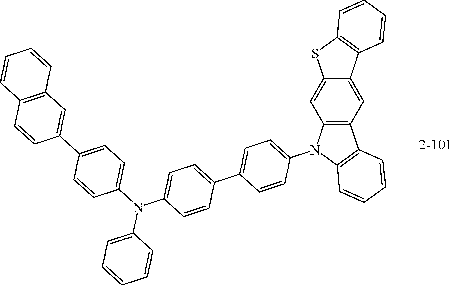 [SMILES: c1ccc(N(c2ccc(-c3ccc(-n4c5ccccc5c5cc6c(cc54)sc4ccccc46)cc3)cc2)c2ccc(-c3ccc4ccccc4c3)cc2)cc1]

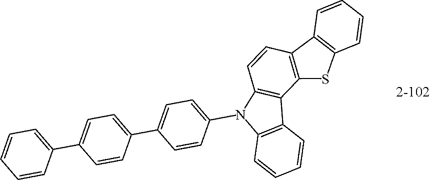 [SMILES: c1ccc(-c2ccc(-c3ccc(-n4c5ccccc5c5c6sc7ccccc7c6ccc54)cc3)cc2)cc1]

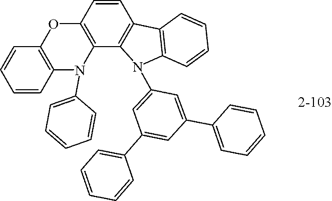 [SMILES: c1ccc(-c2cc(-c3ccccc3)cc(-n3c4ccccc4c4ccc5c(c43)N(c3ccccc3)c3ccccc3O5)c2)cc1]

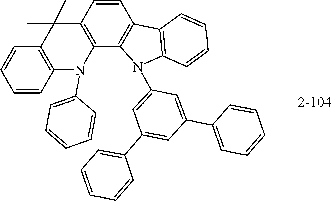 [SMILES: CC1(C)c2ccccc2N(c2ccccc2)c2c1ccc1c3ccccc3n(-c3cc(-c4ccccc4)cc(-c4ccccc4)c3)c21]

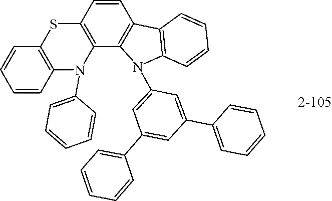 [SMILES: c1ccc(-c2cc(-c3ccccc3)cc(-n3c4ccccc4c4ccc5c(c43)N(c3ccccc3)c3ccccc3S5)c2)cc1]

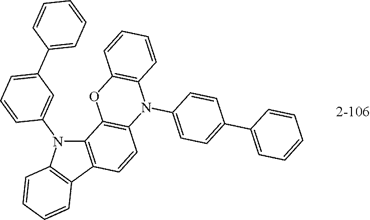 [SMILES: c1ccc(-c2ccc(N3c4ccccc4Oc4c3ccc3c5ccccc5n(-c5cccc(-c6ccccc6)c5)c43)cc2)cc1]

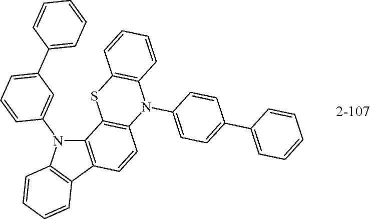 [SMILES: c1ccc(-c2ccc(N3c4ccccc4Sc4c3ccc3c5ccccc5n(-c5cccc(-c6ccccc6)c5)c43)cc2)cc1]

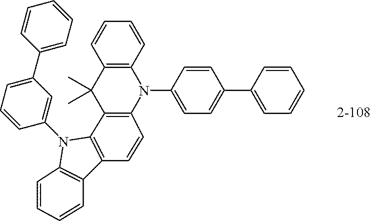 [SMILES: CC1(C)c2ccccc2N(c2ccc(-c3ccccc3)cc2)c2ccc3c4ccccc4n(-c4cccc(-c5ccccc5)c4)c3c21]

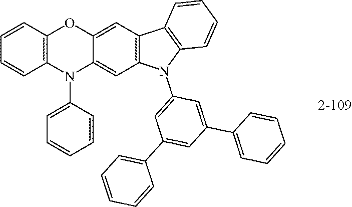 [SMILES: c1ccc(-c2cc(-c3ccccc3)cc(-n3c4ccccc4c4cc5c(cc43)N(c3ccccc3)c3ccccc3O5)c2)cc1]

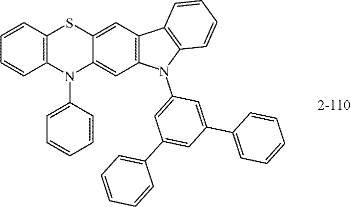 [SMILES: c1ccc(-c2cc(-c3ccccc3)cc(-n3c4ccccc4c4cc5c(cc43)N(c3ccccc3)c3ccccc3S5)c2)cc1]

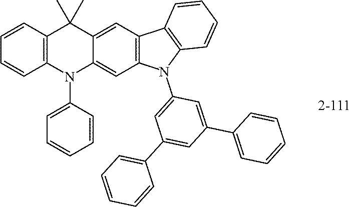 [SMILES: CC1(C)c2ccccc2N(c2ccccc2)c2cc3c(cc21)c1ccccc1n3-c1cc(-c2ccccc2)cc(-c2ccccc2)c1]

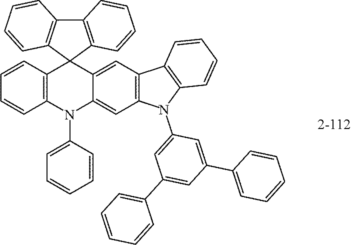 [SMILES: c1ccc(-c2cc(-c3ccccc3)cc(-n3c4ccccc4c4cc5c(cc43)N(c3ccccc3)c3ccccc3C53c4ccccc4-c4ccccc43)c2)cc1]

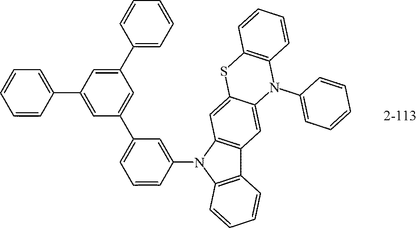 [SMILES: c1ccc(-c2cc(-c3ccccc3)cc(-c3cccc(-n4c5ccccc5c5cc6c(cc54)Sc4ccccc4N6c4ccccc4)c3)c2)cc1]

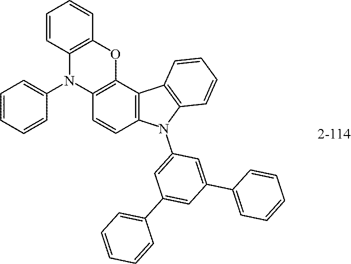 [SMILES: c1ccc(-c2cc(-c3ccccc3)cc(-n3c4ccccc4c4c5c(ccc43)N(c3ccccc3)c3ccccc3O5)c2)cc1]

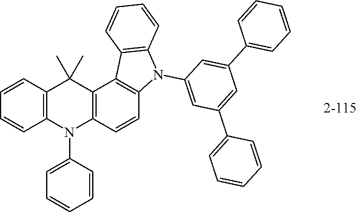 [SMILES: CC1(C)c2ccccc2N(c2ccccc2)c2ccc3c(c21)c1ccccc1n3-c1cc(-c2ccccc2)cc(-c2ccccc2)c1]

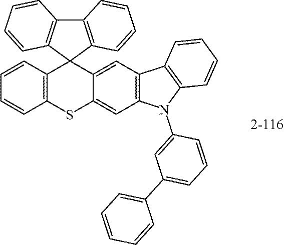 [SMILES: c1ccc(-c2cccc(-n3c4ccccc4c4cc5c(cc43)Sc3ccccc3C53c4ccccc4-c4ccccc43)c2)cc1]

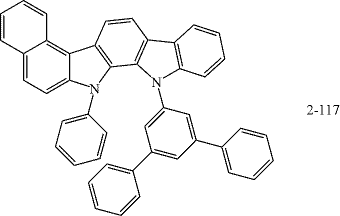 [SMILES: c1ccc(-c2cc(-c3ccccc3)cc(-n3c4ccccc4c4ccc5c6c7ccccc7ccc6n(-c6ccccc6)c5c43)c2)cc1]

In some embodiments, in the mixture as described herein, at least one of the organic compound H1 or the organic compound H2, preferentially the organic compound H1 has ((LUMO+1)-LUMO) ≥0.1 eV, more preferably ≥0.15 eV, even more preferably ≥0.20 eV, further preferably ≥0.25 eV, and most preferably ≥0.30 eV.

In some embodiments, in the mixture as described herein, at least one of the organic compound H1 or the organic compound H2, preferentially the organic compound H2 has (HOMO-(HOMO-1)) ≥0.2 eV, preferably ≥0.25 eV, more preferably ≥0.30 eV, further preferably ≥0.35 eV, and most preferably ≥0.40 eV.

In some embodiments, in the mixture as described herein, the organic compound H1 and the organic compound H2 form a type I heterojunction energy structure, i. e., the highest occupied molecular orbital (HOMO) of the organic compound H1 is higher than the HOMO of the organic compound H2, and the lowest unoccupied molecular orbital (LUMO) of the organic compound H1 is lower than the LUMO of the organic compound H2. Preferably, the organic compound H1 is an organic compound as described herein, and have ΔEₛₜ ≤0.3 eV, preferably ΔEₛₜ ≤0.2 eV, more preferably ΔEₛₜ ≤0.15 eV,, and most preferably ΔEₛₜ ≤0.10 eV.

It is an object of the present disclosure to provide a material for the evaporation-based OLEDs.

In some embodiments, the mixture as described herein are used for evaporation-based OLEDs. For this purpose, the organic compound H₁ and the organic compound H₂ of the organic compound/mixture as described herein have a molecular weight ≤1000 g/mol, preferably ≤900 g/mol, more preferably ≤850 g/mol, further preferably ≤800 g/mol, and most preferably ≤700 g/mol.

In some embodiments, in the mixture as described herein, the molecular weight difference between the organic compound H₁ and the organic compound H₂ ≤100 Daltons, preferably ≤60 Daltons, more preferably ≤30 Daltons.

In some embodiments, in the mixture as described herein, the sublimation temperature difference between the organic compound H₁ and the organic compound H₂ ≤30 K; preferably ≤20 K; more preferably ≤10 K.

Another object of the present disclosure is to provide a material for the printed OLEDs.

For this purpose, at least one of, preferably both the organic compound H₁ and the organic compound H₂ of the organic compound/mixture as described herein have a molecular weight ≥700 g/mol, preferably ≥800 g/mol, more preferably ≥900 g/mol, further preferably ≥1000 g/mol, and most preferably ≥1100 g/mol.

For the evaporation-based OLED, the co-host applied in the form of a Premix requires that the two host materials have similar chemical properties or physical properties, such as molecular weight and sublimation temperature. Meanwhile in solution-processed OLEDs, two host materials with different properties may improve film-forming performance, thereby improving the performance of the devices. The properties mentioned can be glass transition temperature, molecular volumes, etc, in addition to molecular weight, sublimation temperature. For printing OLEDs, further preferred embodiments of the organic mixture as described herein include:

1) The molecular weight difference between the organic compound H₁ and the organic compound H₂ ≥120 g/mol, preferably ≥140 g/mol, more preferably ≥160 g/mol, and most preferably ≥180 g/mol.

2) The sublimation temperature difference between the organic compound H₁ and the organic compound H₂ ≥60 K, preferably ≥70 K, more preferably ≥75 K, and most preferably ≥80 K.

3) The glass transition temperature difference between the organic compound H₁ and the organic compound H₂ ≥20 K, preferably ≥30 K, more preferably ≥40 K, and most preferably ≥45 K.

4) The molecular volume difference between the organic compound H₁ and the organic compound H₂ ≥20%, preferably ≥30%, more preferably ≥40%, and most preferably ≥45%.

In some embodiments, at least one of, preferably both the organic compound H₁ and the organic compound H₂ of the organic compound/mixture as described herein has a solubility of ≥2 mg/mL in toluene at 25° C., preferably ≥3 mg/mL, more preferably ≥4 mg/mL, and most preferably ≥5 mg/mL.

In some embodiments, in the mixture as described herein, the molar ratio of the organic compound H1 and the organic compound H2 is from 2:8 to 8:2; the preferred molar ratio is from 3:7 to 7:3; the more preferred molar ratio is from 4:6 to 6:4.

The phosphorescent host material (triplet host), phosphorescent emitter (triplet emitter), TADF material, and HTM are described in detail below (but not limited thereto).

### 1. Triplet Host

Examples of triplet host material are not particularly limited and any metal complex or organic compound may be used as the host material as long as its triplet energy is greater than that of the light emitter, especially a triplet emitter or phosphorescent emitter. Examples of metal complexes that may be used as triplet hosts may include, but are not limited to, the general structure as follows:

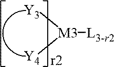

Where M3 is a metal; (Y³ - Y⁴) is a bidentate ligand, Y³ and Y⁴ are independently selected from C, N, O, P, or S; L is an auxiliary ligand; r2 is an integer with the value from 1 to the maximum coordination number of the metal.

In some embodiments, the metal complex which may be used as the triplet host have the following form:

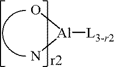

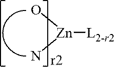

(O—N) is a bidentate ligand in which the metal is coordinated to O and N atoms, r2 is an integer with the value from 1 to the maximum coordination number of the metal.

In some embodiments, M3 may be selected from Ir and Pt.

Examples of organic compounds used as triplet hosts are selected from: compounds comprising cyclic aryl groups, such as benzene, biphenyl, triphenyl, benzofluorene; compounds comprising heterocyclic aryl groups, such as dibenzothiophene, dibenzofuran, dibenzoselenophene, furan, thiophene, benzofuran, benzothiophene, benzoselenophene, carbazole, dibenzocarbazole, indocarbazole, pyridoindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, oxazole, dibenzooxazole, benzisoxazole, benzothiazole, quinoline, isoquinoline, o-diazonaphthalene, quinazoline, quinoxaline, naphthalene, phthalein, pteridine, xanthene, acridine, phenazine, phenothiazine, phenoxazine, dibenzoselenophene, benzofuranopyridine, furazopyridine, benzothienopyridine, thienodipyridine, benzoselenophenopyridine, and selenophenodipyridine; and groups comprising 2 to 10 ring structures which may be the same or different types of cyclic aryl or aromatic heterocyclic group and are bonded to each other directly or through at least one of the following groups: oxygen atom, nitrogen atom, sulfur atom, silicon atom, phosphorus atom, boron atom, chain structural unit, and aliphatic cyclic group.

In some embodiments, the triplet host material may be selected from the compounds comprising at least one of the following groups:

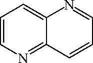

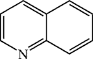

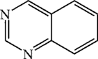

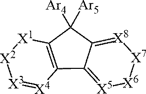

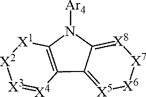

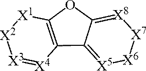

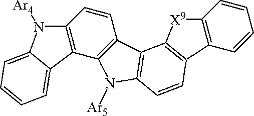

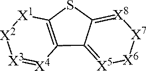

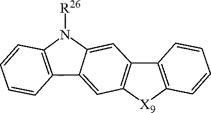

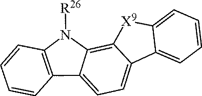

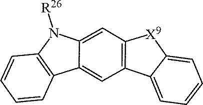

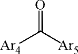

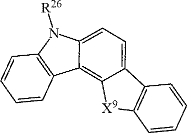

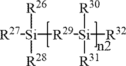

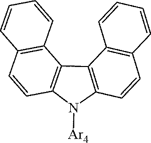

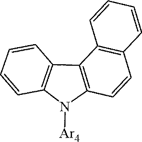

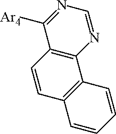

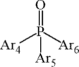

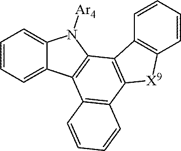

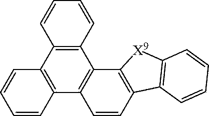

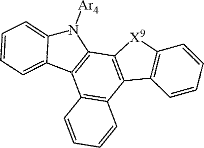

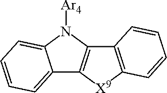

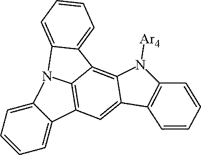

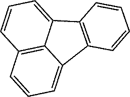 [SMILES: c1ccc2c(c1)-c1cccc3cccc-2c13]

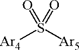

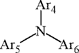

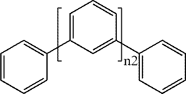

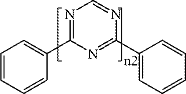

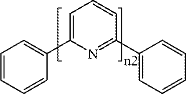

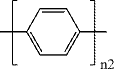

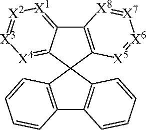

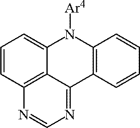

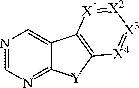

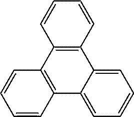 [SMILES: c1ccc2c(c1)c1ccccc1c1ccccc21]

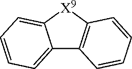

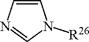

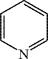

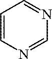

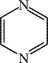

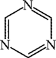

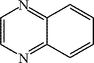

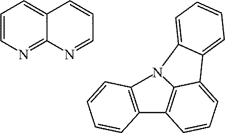 [SMILES: c1cnc2ncccc2c1 ; c1ccc2c(c1)c1cccc3c4ccccc4n2c13]

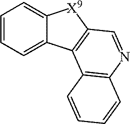

X⁹ is CR³³R³⁴ or NR³⁵; Y is CR³⁶R³⁷, NR³⁸, O, or S; R²⁶ to R³⁸ are the same defined as above-mentioned R¹; n2 is an integer from 1 to 3; X¹ to X⁸ are identically defined as described above; Ar₄ to Ar₆ are identically defined as the above-mentioned Ar₁.

Examples of suitable triplet host materials are listed below, but not limited to:

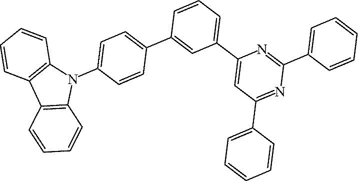 [SMILES: c1ccc(-c2cc(-c3cccc(-c4ccc(-n5c6ccccc6c6ccccc65)cc4)c3)nc(-c3ccccc3)n2)cc1]

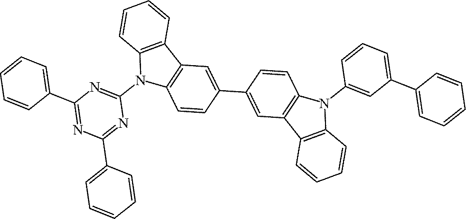 [SMILES: c1ccc(-c2cccc(-n3c4ccccc4c4cc(-c5ccc6c(c5)c5ccccc5n6-c5nc(-c6ccccc6)nc(-c6ccccc6)n5)ccc43)c2)cc1]

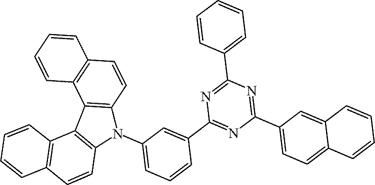 [SMILES: c1ccc(-c2nc(-c3cccc(-n4c5ccc6ccccc6c5c5c6ccccc6ccc54)c3)nc(-c3ccc4ccccc4c3)n2)cc1]

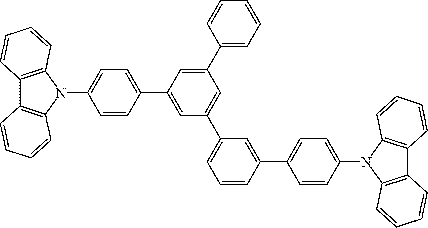 [SMILES: c1ccc(-c2cc(-c3ccc(-n4c5ccccc5c5ccccc54)cc3)cc(-c3cccc(-c4ccc(-n5c6ccccc6c6ccccc65)cc4)c3)c2)cc1]

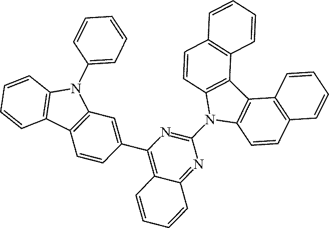 [SMILES: c1ccc(-n2c3ccccc3c3ccc(-c4nc(-n5c6ccc7ccccc7c6c6c7ccccc7ccc65)nc5ccccc45)cc32)cc1]

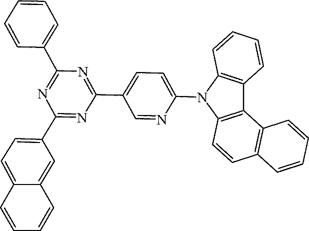 [SMILES: c1ccc(-c2nc(-c3ccc(-n4c5ccccc5c5c6ccccc6ccc54)nc3)nc(-c3ccc4ccccc4c3)n2)cc1]

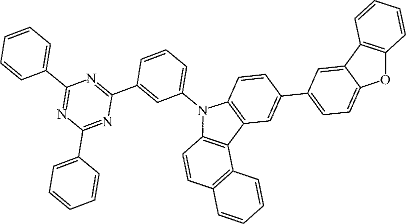 [SMILES: c1ccc(-c2nc(-c3ccccc3)nc(-c3cccc(-n4c5ccc(-c6ccc7oc8ccccc8c7c6)cc5c5c6ccccc6ccc54)c3)n2)cc1]

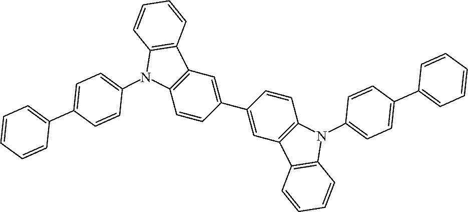 [SMILES: c1ccc(-c2ccc(-n3c4ccccc4c4cc(-c5ccc6c(c5)c5ccccc5n6-c5ccc(-c6ccccc6)cc5)ccc43)cc2)cc1]

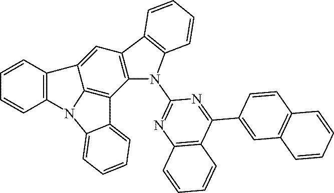 [SMILES: c1ccc2cc(-c3nc(-n4c5ccccc5c5cc6c7ccccc7n7c8ccccc8c(c54)c67)nc4ccccc34)ccc2c1]

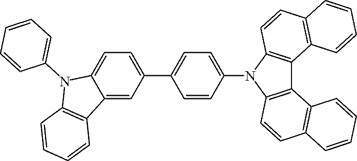 [SMILES: c1ccc(-n2c3ccccc3c3cc(-c4ccc(-n5c6ccc7ccccc7c6c6c7ccccc7ccc65)cc4)ccc32)cc1]

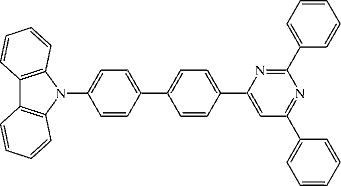 [SMILES: c1ccc(-c2cc(-c3ccc(-c4ccc(-n5c6ccccc6c6ccccc65)cc4)cc3)nc(-c3ccccc3)n2)cc1]

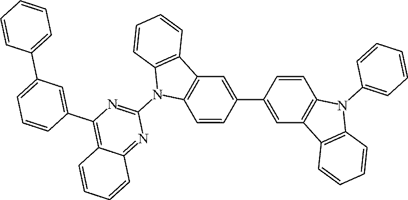 [SMILES: c1ccc(-c2cccc(-c3nc(-n4c5ccccc5c5cc(-c6ccc7c(c6)c6ccccc6n7-c6ccccc6)ccc54)nc4ccccc34)c2)cc1]

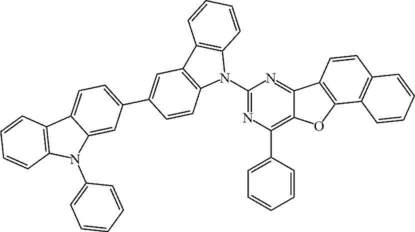 [SMILES: c1ccc(-c2nc(-n3c4ccccc4c4cc(-c5ccc6c7ccccc7n(-c7ccccc7)c6c5)ccc43)nc3c2oc2c4ccccc4ccc32)cc1]

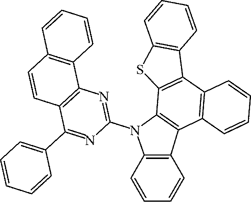 [SMILES: c1ccc(-c2nc(-n3c4ccccc4c4c5ccccc5c5c6ccccc6sc5c43)nc3c2ccc2ccccc23)cc1]

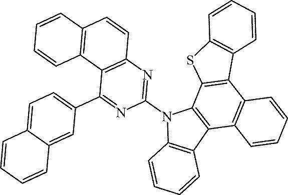 [SMILES: c1ccc2cc(-c3nc(-n4c5ccccc5c5c6ccccc6c6c7ccccc7sc6c54)nc4ccc5ccccc5c34)ccc2c1]

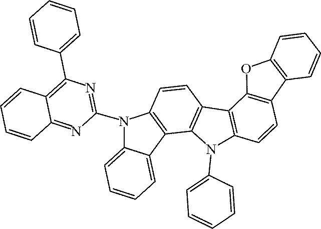 [SMILES: c1ccc(-c2nc(-n3c4ccccc4c4c3ccc3c5c6oc7ccccc7c6ccc5n(-c5ccccc5)c34)nc3ccccc23)cc1]

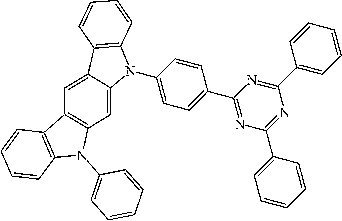 [SMILES: c1ccc(-c2nc(-c3ccccc3)nc(-c3ccc(-n4c5ccccc5c5cc6c7ccccc7n(-c7ccccc7)c6cc54)cc3)n2)cc1]

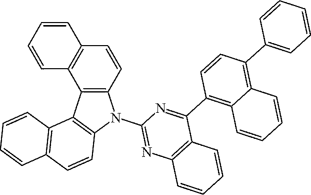 [SMILES: c1ccc(-c2ccc(-c3nc(-n4c5ccc6ccccc6c5c5c6ccccc6ccc54)nc4ccccc34)c3ccccc23)cc1]

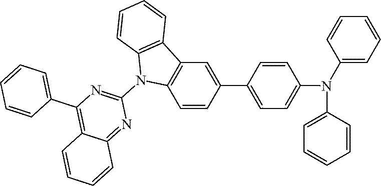 [SMILES: c1ccc(-c2nc(-n3c4ccccc4c4cc(-c5ccc(N(c6ccccc6)c6ccccc6)cc5)ccc43)nc3ccccc23)cc1]

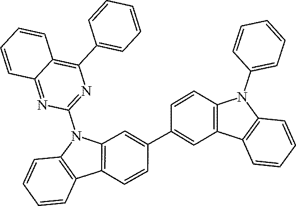 [SMILES: c1ccc(-c2nc(-n3c4ccccc4c4ccc(-c5ccc6c(c5)c5ccccc5n6-c5ccccc5)cc43)nc3ccccc23)cc1]

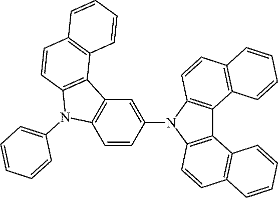 [SMILES: c1ccc(-n2c3ccc(-n4c5ccc6ccccc6c5c5c6ccccc6ccc54)cc3c3c4ccccc4ccc32)cc1]

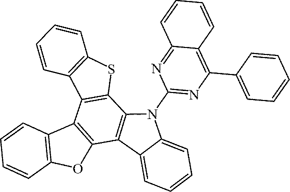 [SMILES: c1ccc(-c2nc(-n3c4ccccc4c4c5oc6ccccc6c5c5c6ccccc6sc5c43)nc3ccccc23)cc1]

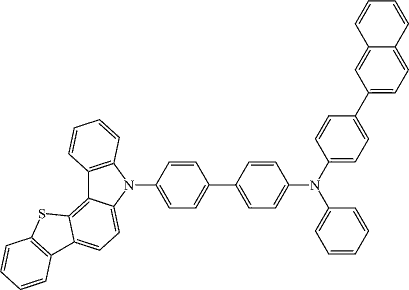 [SMILES: c1ccc(N(c2ccc(-c3ccc(-n4c5ccccc5c5c6sc7ccccc7c6ccc54)cc3)cc2)c2ccc(-c3ccc4ccccc4c3)cc2)cc1]

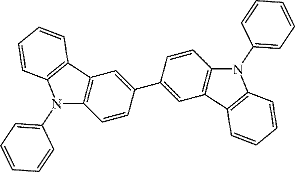 [SMILES: c1ccc(-n2c3ccccc3c3cc(-c4ccc5c(c4)c4ccccc4n5-c4ccccc4)ccc32)cc1]

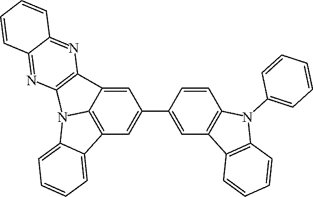 [SMILES: c1ccc(-n2c3ccccc3c3cc(-c4cc5c6ccccc6n6c7nc8ccccc8nc7c(c4)c56)ccc32)cc1]

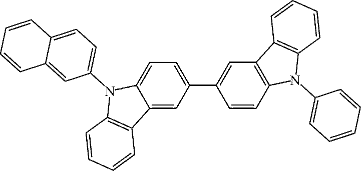 [SMILES: c1ccc(-n2c3ccccc3c3cc(-c4ccc5c(c4)c4ccccc4n5-c4ccc5ccccc5c4)ccc32)cc1]

### 2. Triplet Emitter

The triplet emitter is also called a phosphorescent emitter. In some embodiments, the triplet emitter is a metal complex of formula M(L)ₙ, where M may be a metal atom; L may be a same or different organic ligand each time it is present, and may be bonded or coordinated to the metal atom M at one or more positions; n is an integer greater than 1, preferably is 1, 2, 3, 4, 5, or 6. Alternatively, these metal complexes may be attached to a polymer by one or more positions, most preferably through an organic ligand.

In some embodiments, the metal atom M may be selected from the group consisting of transition metal elements or lanthanides or actinides, preferably Ir, Pt, Pd, Au, Rh, Ru, Os, Sm, Eu, Gd, Tb, Dy, Re, Cu or Ag, and particularly preferably Os, Ir, Ru, Rh, Re, Pd, Au, or Pt.

Preferably, the triplet emitter comprises a chelating ligand (i.e., a ligand) coordinating to the metal by at least two bonding sites, and it is particularly preferred that the triplet emitter comprises two or three identical or different bidentate or multidentate ligand. Chelating ligands help to improve stability of metal complexes.

Examples of organic ligands may be selected from the group consisting of phenylpyridine derivatives, 7,8-benzoquinoline derivatives, 2 (2-thienyl) pyridine derivatives, 2 (1-naphthyl) pyridine derivatives, or 2 phenylquinoline derivatives. All of these organic ligands may be substituted, for example, substituted with fluoromethyl or trifluoromethyl. The auxiliary ligand may be preferably selected from acetylacetonate or picric acid.

In some embodiments, the metal complex which may be used as the triplet emitter may have the following form:

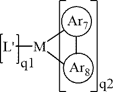

Where M is a metal selected from the group consisting of transition metal elements or lanthanides or actinides, and particularly preferably Ir, Pt, Au.

Ar₇ may be the same or different cyclic group each time it is present, which comprises a donor atom, that is an atom with a lone pair of electrons, such as nitrogen atom or phosphorus atom, which is coordinated to the metal through its ring group; Ar₈ may be the same or different cyclic group comprising a C atom and is coordinated to the metal through its ring group; Ar₇ and Ar₈ are covalently bonded together, where each of them may carry one or more substituents which may also be joined together by substituents; L′ may be the same or different at each occurrence and is a bidentate chelating ligand, and most preferably a monoanionic bidentate chelating ligand; q1 is 1, 2 or 3, preferably 2 or 3; q2 is 0, 1, 2, or 3, and preferably 0 or 1.

Examples of triplet emitter materials that are extremely useful may be found in the following patent documents and references: WO200070655, WO200141512, WO200202714, WO200215645, EP1191613, EP1191612, EP1191614, WO2005033244, WO2005019373, US20050258742, WO2009146770, WO2010015307, WO2010031485, WO2010054731, WO2010054728, WO2010086089, WO2010099852, WO2010102709, US20070087219A1, US20090061681A1, US20010053462A1, Baldo, Thompson et al. Nature 403, (2000), 750-753, Adachi et al. Appl. Phys. Lett. 78 (2001),1622-1624, J. Kido et al. Appl. Phys. Lett. 65 (1994), 2124, Kido et al. Chem. Lett. 657, 1990, US20070252517A1, Johnson et al., JACS 105, 1983, 1795, Wrighton, JACS 96, 1974, 998, Ma et al., Synth. Metals 94, 1998, 245, US6824895, US7029766, US6835469, US6830828, US20010053462A1, WO2007095118A1, US2012004407A1, WO2012007088A1, WO2012007087A1, WO2012007086A1, US2008027220A1, WO2011157339A1, CN102282150A, WO2009118087A1, WO2013107487A1, WO2013094620A1, WO2013174471A1, WO2014031977A1, WO2014112450A1, WO2014007565A1, WO2014038456A1, WO2014024131A1, WO2014008982A1, WO201402337A1. The patent documents listed above are specially incorporated herein by reference in their entirety.

Examples of some suitable triplet emitters are listed below:

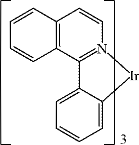

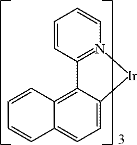

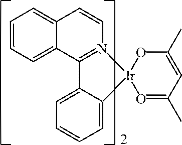

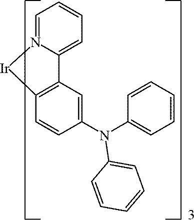

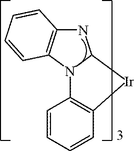

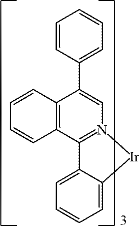

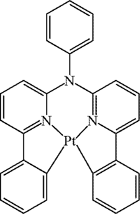

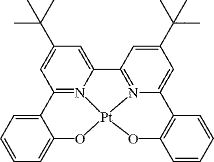

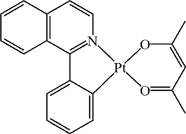

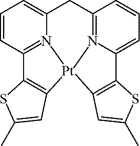

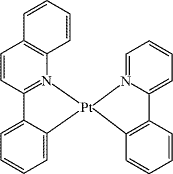

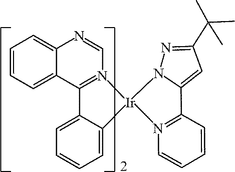

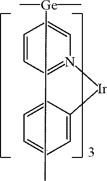

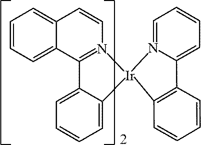

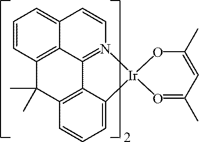

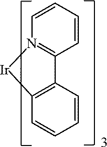

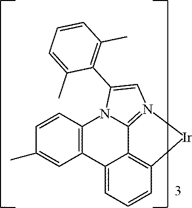

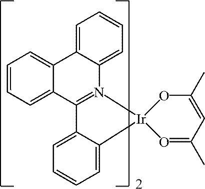

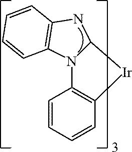

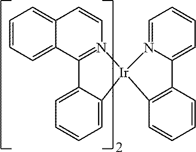

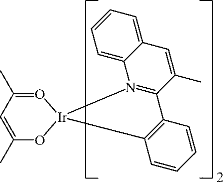

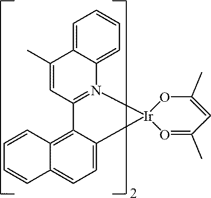

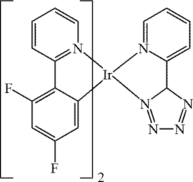

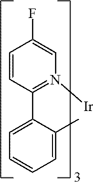

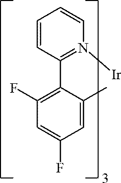

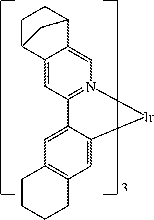

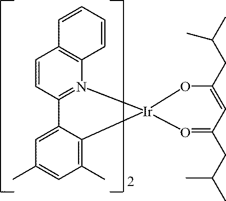

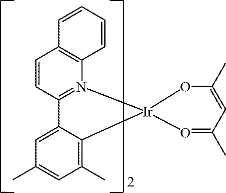

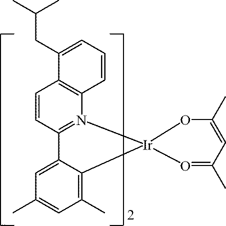

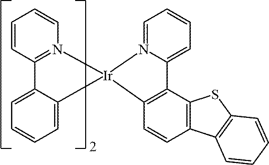

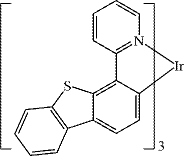

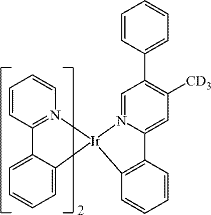

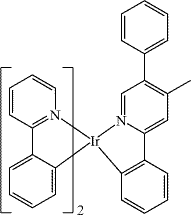

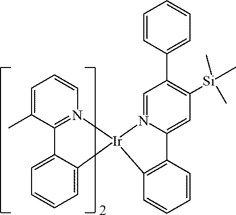

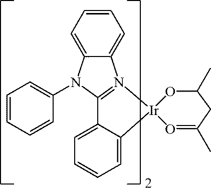

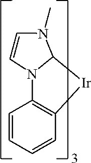

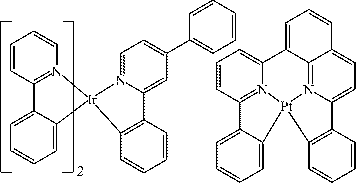

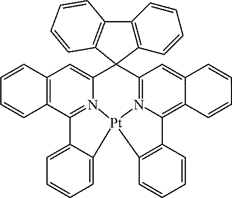

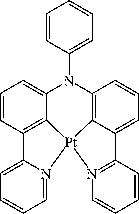

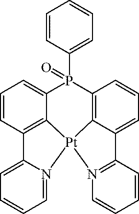

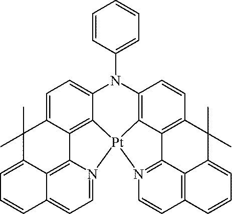

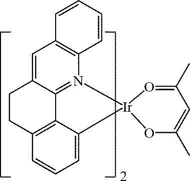

### 3. TADF Material

Conventional organic fluorescent materials can only emit light using 25% singlet excitonic luminescence formed by electrical excitation, and the devices have relatively low internal quantum efficiency (up to 25%). The phosphorescent material enhances the intersystem crossing due to the strong spin-orbit coupling of the heavy atom center, the singlet exciton and the triplet exciton luminescence formed by the electric excitation can be effectively utilized, so that the internal quantum efficiency of the device can reach 100%. However, the phosphorescent materials are expensive, the material stability is poor, and the device efficiency roll-off is a serious problem, which limit its application in OLED. Thermally-activated delayed fluorescent (TADF) materials are the third generation of organic light-emitting materials developed after organic fluorescent materials and organic phosphorescent materials. This type of material generally has a small singlet-triplet energy level difference (ΔEst), and triplet excitons can be converted to singlet excitons by intersystem crossing. This can make full use of the singlet excitons and triplet excitons formed under electric excitation. The device can achieve 100% internal quantum efficiency. Meanwhile, due to the controllable structure, stable property, low cost, TADF materials without precious metals have a wide application prospect in the OLED field.

The TADF material needs to have a small singlet-triplet energy level difference, preferably ΔEst <0.3 eV, more preferably ΔEst <0.2 eV, and most preferably ΔEst <0.1 eV. In some embodiments, the TADF material has a relatively small ΔEst. In some embodiments, the TADF has a high fluorescence quantum efficiency. Some TADF materials can be found in the following patent documents: CN103483332A, TW201309696A, TW201309778A, TW201343874A, TW201350558A, US20120217869A1, WO2013133359A1, WO2013154064A1, Adachi, et. al. Adv. Mater., 21, 2009, 4802, Adachi, et. al. Appl. Phys. Lett., 98, 2011, 083302, Adachi, et. al. Appl. Phys. Lett., 101, 2012, 093306, Adachi, et. al. Chem. Commun., 48, 2012, 11392, Adachi, et. al. Nature Photonics, 6, 2012, 253, Adachi, et. al. Nature, 492, 2012, 234, Adachi, et. al. J. Am. Chem. Soc,134, 2012, 14706, Adachi, et. al. Angew. Chem. Int. Ed, 51, 2012, 11311, Adachi, et. al. Chem. Commun., 48, 2012, 9580, Adachi, et. al. Chem. Commun., 48, 2013, 10385, Adachi, et. al. Adv. Mater., 25, 2013, 3319, Adachi, et. al. Adv. Mater., 25, 2013, 3707, Adachi, et. al. Chem. Mater., 25, 2013, 3038, Adachi, et. al. Chem. Mater., 25, 2013,3766, Adachi, et. al. J. Mater. Chem. C., 1, 2013, 4599, Adachi, et. al. J. Phys. Chem. A., 117, 2013, 5607. The entire contents of the above listed patents or literature documents are hereby incorporated by reference.

Examples of some suitable TADF materials are listed below:

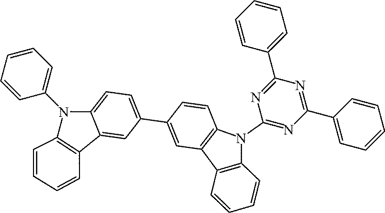 [SMILES: c1ccc(-c2nc(-c3ccccc3)nc(-n3c4ccccc4c4cc(-c5ccc6c(c5)c5ccccc5n6-c5ccccc5)ccc43)n2)cc1]

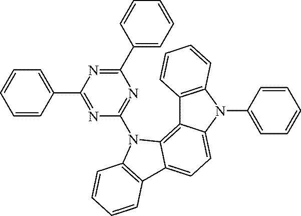 [SMILES: c1ccc(-c2nc(-c3ccccc3)nc(-n3c4ccccc4c4ccc5c(c6ccccc6n5-c5ccccc5)c43)n2)cc1]

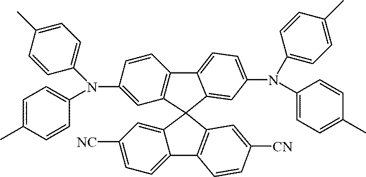

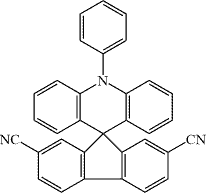

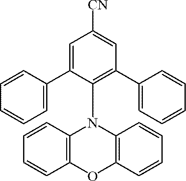 [SMILES: N#Cc1cc(-c2ccccc2)c(N2c3ccccc3Oc3ccccc32)c(-c2ccccc2)c1]

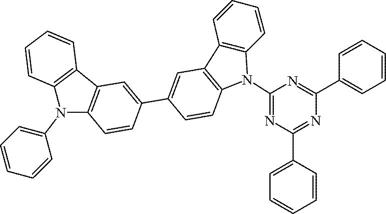 [SMILES: c1ccc(-c2nc(-c3ccccc3)nc(-n3c4ccccc4c4cc(-c5ccc6c(c5)c5ccccc5n6-c5ccccc5)ccc43)n2)cc1]

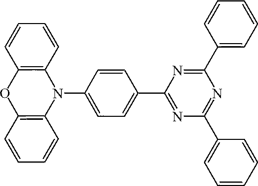 [SMILES: c1ccc(-c2nc(-c3ccccc3)nc(-c3ccc(N4c5ccccc5Oc5ccccc54)cc3)n2)cc1]

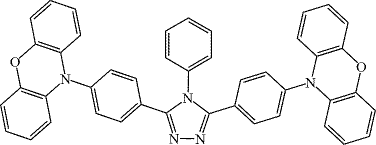 [SMILES: c1ccc(-n2c(-c3ccc(N4c5ccccc5Oc5ccccc54)cc3)nnc2-c2ccc(N3c4ccccc4Oc4ccccc43)cc2)cc1]

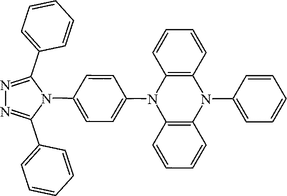 [SMILES: c1ccc(-c2nnc(-c3ccccc3)n2-c2ccc(N3c4ccccc4N(c4ccccc4)c4ccccc43)cc2)cc1]

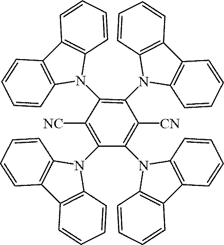

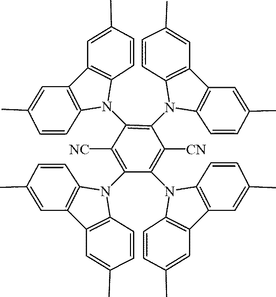

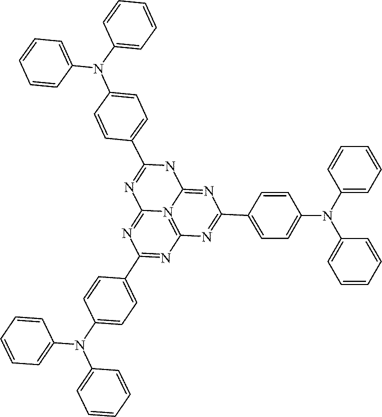

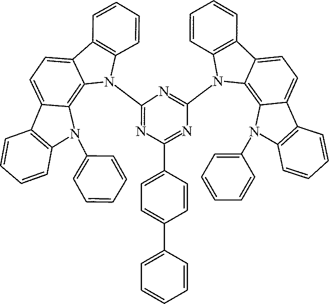 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1]

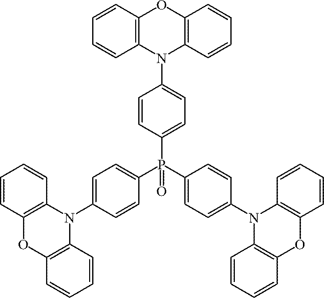 [SMILES: O=P(c1ccc(N2c3ccccc3Oc3ccccc32)cc1)(c1ccc(N2c3ccccc3Oc3ccccc32)cc1)c1ccc(N2c3ccccc3Oc3ccccc32)cc1]

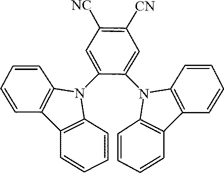

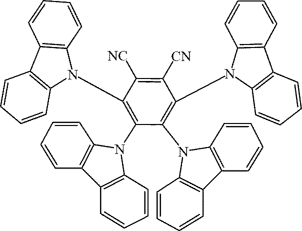

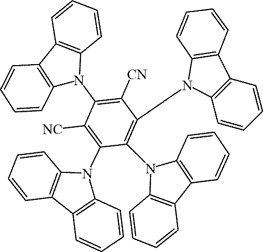

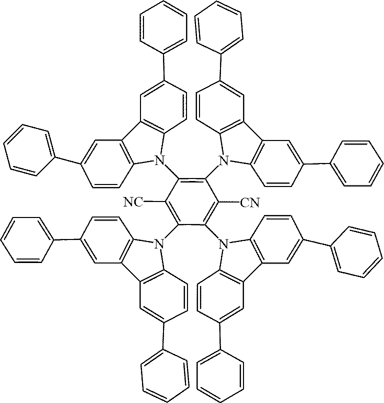

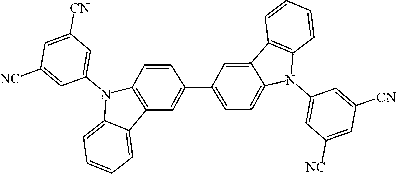

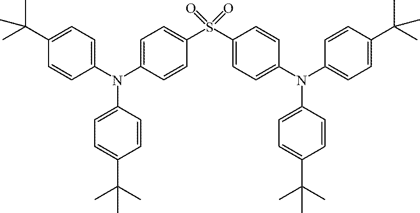 [SMILES: CC(C)(C)c1ccc(N(c2ccc(C(C)(C)C)cc2)c2ccc(S(=O)(=O)c3ccc(N(c4ccc(C(C)(C)C)cc4)c4ccc(C(C)(C)C)cc4)cc3)cc2)cc1]

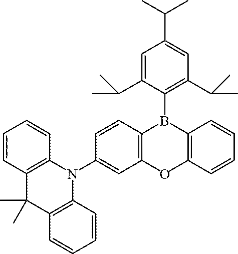 [SMILES: CC(C)c1cc(C(C)C)c(B2c3ccccc3Oc3cc(N4c5ccccc5C(C)(C)c5ccccc54)ccc32)c(C(C)C)c1]

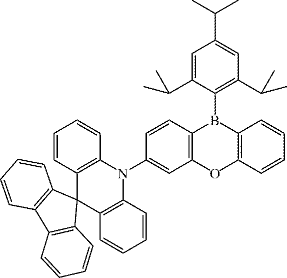 [SMILES: CC(C)c1cc(C(C)C)c(B2c3ccccc3Oc3cc(N4c5ccccc5C5(c6ccccc6-c6ccccc65)c5ccccc54)ccc32)c(C(C)C)c1]

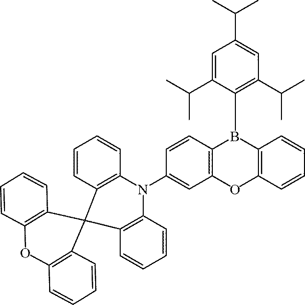 [SMILES: CC(C)c1cc(C(C)C)c(B2c3ccccc3Oc3cc(N4c5ccccc5C5(c6ccccc6Oc6ccccc65)c5ccccc54)ccc32)c(C(C)C)c1]

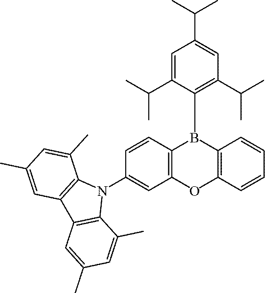 [SMILES: Cc1cc(C)c2c(c1)c1cc(C)cc(C)c1n2-c1ccc2c(c1)Oc1ccccc1B2c1c(C(C)C)cc(C(C)C)cc1C(C)C]

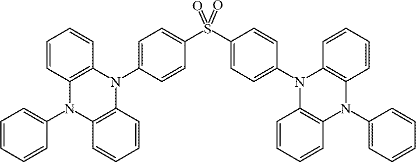 [SMILES: O=S(=O)(c1ccc(N2c3ccccc3N(c3ccccc3)c3ccccc32)cc1)c1ccc(N2c3ccccc3N(c3ccccc3)c3ccccc32)cc1]

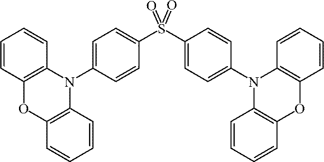 [SMILES: O=S(=O)(c1ccc(N2c3ccccc3Oc3ccccc32)cc1)c1ccc(N2c3ccccc3Oc3ccccc32)cc1]

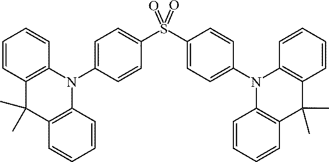

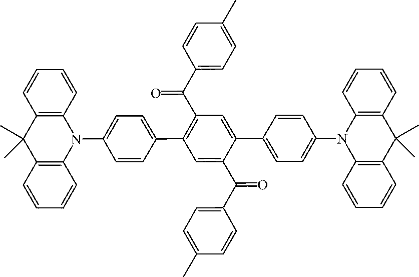

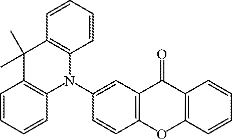 [SMILES: CC1(C)c2ccccc2N(c2ccc3oc4ccccc4c(=O)c3c2)c2ccccc21]

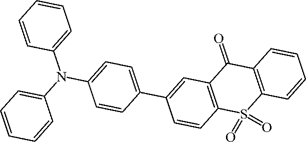 [SMILES: O=C1c2ccccc2S(=O)(=O)c2ccc(-c3ccc(N(c4ccccc4)c4ccccc4)cc3)cc21]

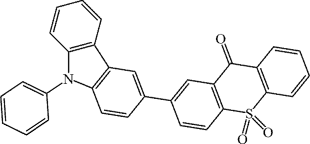 [SMILES: O=C1c2ccccc2S(=O)(=O)c2ccc(-c3ccc4c(c3)c3ccccc3n4-c3ccccc3)cc21]

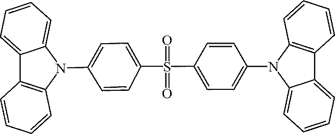 [SMILES: O=S(=O)(c1ccc(-n2c3ccccc3c3ccccc32)cc1)c1ccc(-n2c3ccccc3c3ccccc32)cc1]

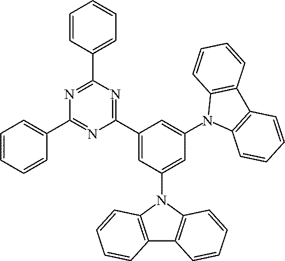 [SMILES: c1ccc(-c2nc(-c3ccccc3)nc(-c3cc(-n4c5ccccc5c5ccccc54)cc(-n4c5ccccc5c5ccccc54)c3)n2)cc1]

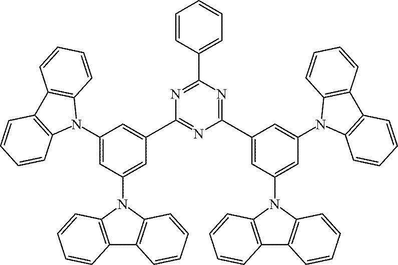 [SMILES: c1ccc(-c2nc(-c3cc(-n4c5ccccc5c5ccccc54)cc(-n4c5ccccc5c5ccccc54)c3)nc(-c3cc(-n4c5ccccc5c5ccccc54)cc(-n4c5ccccc5c5ccccc54)c3)n2)cc1]

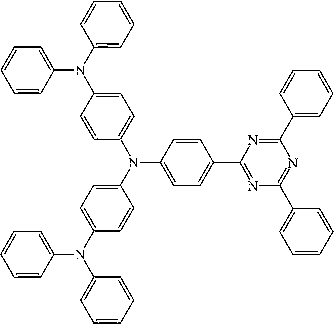 [SMILES: c1ccc(-c2nc(-c3ccccc3)nc(-c3ccc(N(c4ccc(N(c5ccccc5)c5ccccc5)cc4)c4ccc(N(c5ccccc5)c5ccccc5)cc4)cc3)n2)cc1]

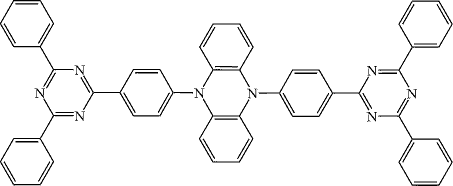 [SMILES: c1ccc(-c2nc(-c3ccccc3)nc(-c3ccc(N4c5ccccc5N(c5ccc(-c6nc(-c7ccccc7)nc(-c7ccccc7)n6)cc5)c5ccccc54)cc3)n2)cc1]

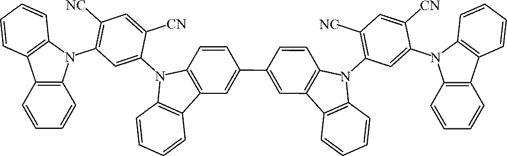

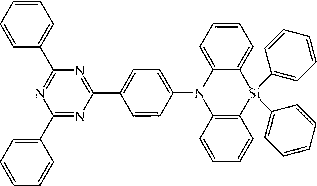 [SMILES: c1ccc(-c2nc(-c3ccccc3)nc(-c3ccc(N4c5ccccc5[Si](c5ccccc5)(c5ccccc5)c5ccccc54)cc3)n2)cc1]

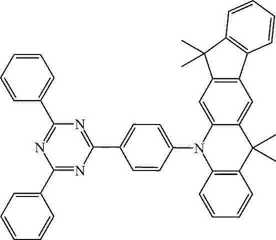 [SMILES: CC1(C)c2ccccc2-c2cc3c(cc21)N(c1ccc(-c2nc(-c4ccccc4)nc(-c4ccccc4)n2)cc1)c1ccccc1C3(C)C]

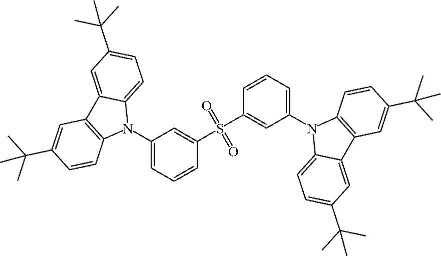 [SMILES: CC(C)(C)c1ccc2c(c1)c1cc(C(C)(C)C)ccc1n2-c1cccc(S(=O)(=O)c2cccc(-n3c4ccc(C(C)(C)C)cc4c4cc(C(C)(C)C)ccc43)c2)c1]

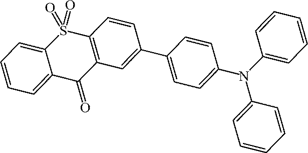 [SMILES: O=C1c2ccccc2S(=O)(=O)c2ccc(-c3ccc(N(c4ccccc4)c4ccccc4)cc3)cc21]

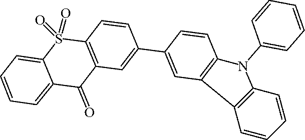 [SMILES: O=C1c2ccccc2S(=O)(=O)c2ccc(-c3ccc4c(c3)c3ccccc3n4-c3ccccc3)cc21]

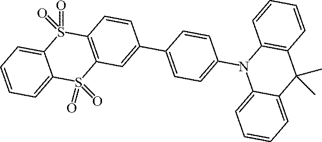 [SMILES: CC1(C)c2ccccc2N(c2ccc(-c3ccc4c(c3)S(=O)(=O)c3ccccc3S4(=O)=O)cc2)c2ccccc21]

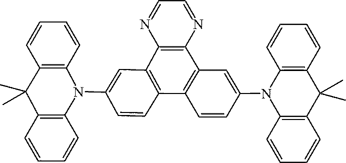

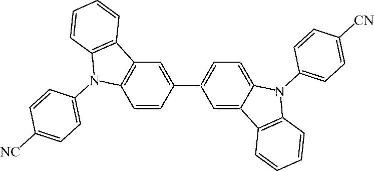

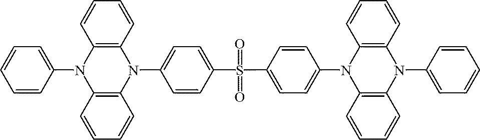 [SMILES: O=S(=O)(c1ccc(N2c3ccccc3N(c3ccccc3)c3ccccc32)cc1)c1ccc(N2c3ccccc3N(c3ccccc3)c3ccccc32)cc1]

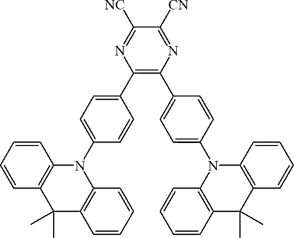

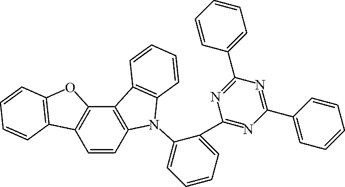 [SMILES: c1ccc(-c2nc(-c3ccccc3)nc(-c3ccccc3-n3c4ccccc4c4c5oc6ccccc6c5ccc43)n2)cc1]

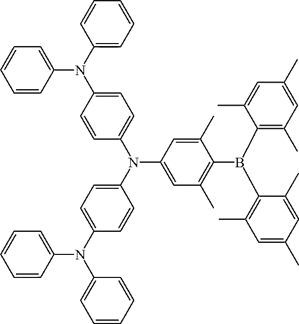 [SMILES: Cc1cc(C)c(B(c2c(C)cc(C)cc2C)c2c(C)cc(N(c3ccc(N(c4ccccc4)c4ccccc4)cc3)c3ccc(N(c4ccccc4)c4ccccc4)cc3)cc2C)c(C)c1]

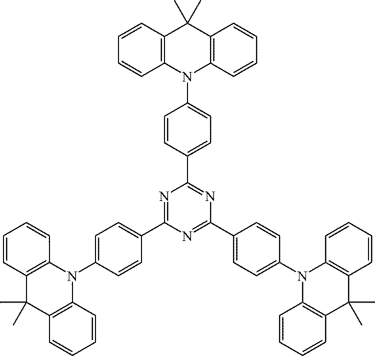

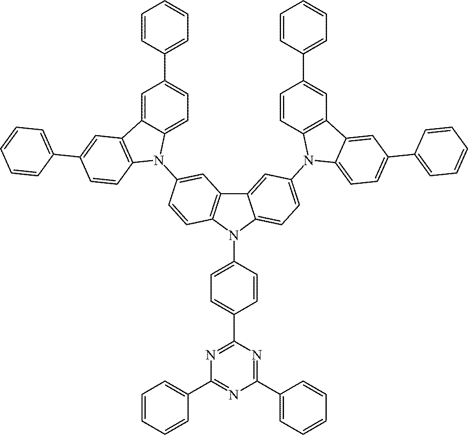 [SMILES: c1ccc(-c2ccc3c(c2)c2cc(-c4ccccc4)ccc2n3-c2ccc3c(c2)c2cc(-n4c5ccc(-c6ccccc6)cc5c5cc(-c6ccccc6)ccc54)ccc2n3-c2ccc(-c3nc(-c4ccccc4)nc(-c4ccccc4)n3)cc2)cc1]

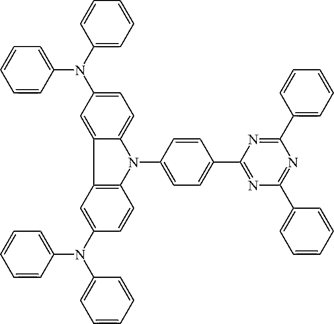 [SMILES: c1ccc(-c2nc(-c3ccccc3)nc(-c3ccc(-n4c5ccc(N(c6ccccc6)c6ccccc6)cc5c5cc(N(c6ccccc6)c6ccccc6)ccc54)cc3)n2)cc1]

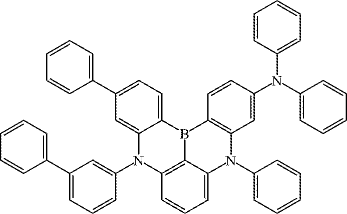 [SMILES: c1ccc(-c2cccc(N3c4cc(-c5ccccc5)ccc4B4c5ccc(N(c6ccccc6)c6ccccc6)cc5N(c5ccccc5)c5cccc3c54)c2)cc1]

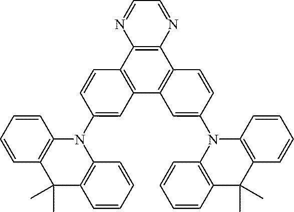 [SMILES: CC1(C)c2ccccc2N(c2ccc3c(c2)c2cc(N4c5ccccc5C(C)(C)c5ccccc54)ccc2c2nccnc32)c2ccccc21]

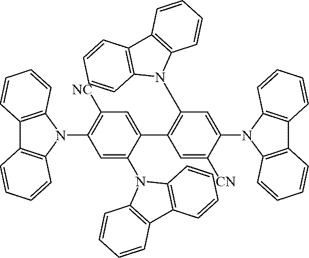

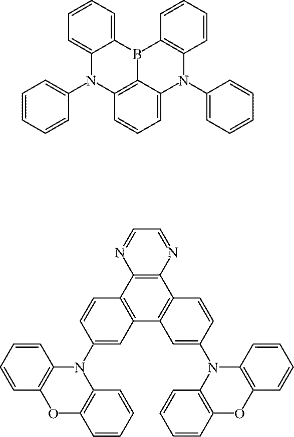 [SMILES: c1ccc(N2c3ccccc3B3c4ccccc4N(c4ccccc4)c4cccc2c43)cc1 ; c1ccc2c(c1)Oc1ccccc1N2c1ccc2c(c1)c1cc(N3c4ccccc4Oc4ccccc43)ccc1c1nccnc21]

### 4. Htm

Suitable organic HIM materials may include any one or any combination of the compounds having the following structural units: phthalocyanines, porphyrins, amines, aryl amines, biphenyl triaryl amines, thiophenes, thiophenes, pyrroles, anilines, carbazoles, indenofluorenes, and derivatives thereof.

Examples of cyclic aromatic amine-derived compounds that can be used as HTM include, but are not limited to, the general structure as follows:

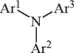

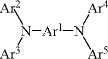

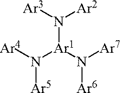

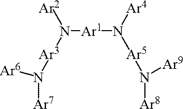

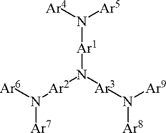

Where each Ar¹ to Ar⁹ may be independently selected from: cyclic aryl groups such as benzene, biphenyl, triphenyl, benzo, naphthalene, anthracene, phenalene, phenanthrene, fluorene, pyrene, chrysene, perylene, azulene; and aromatic heterocyclic groups such as dibenzothiophene,dibenzofuran, furan, thiophene, benzofuran, benzothiophene, benzoselenophene, carbazole, pyrazole, imidazole,triazole, isoxazole, thiazole, oxadiazole, oxatriazole, dioxazole, thiadiazole, pyridine, pyridazine, pyrimidine, pyrazine, triazine, oxazine, oxathiazin, oxadiazine, indole, benzimidazole, indazole, indoxazine, bisbenzoxazole, benzisoxazole, benzothiazole, quinoline, isoquinoline, cinnoline, quinazoline, quinoxaline, naphthalene, phthalein, pteridine, xanthene, acridine, phenazine, phenothiazine, phenoxazine, dibenzoselenophene, benzoselenophene, benzofuropyridine, indolocarbazole, pyridylindole, pyrrolodipyridine, furodipyridine, benzothienopyridine, thienodipyridine, benzoselenophenopyridine, and selenophenodipyridine; groups comprising 2 to 10 ring structures which may be the same or different types of cyclic aryl or aromatic heterocyclic group and are bonded to each other directly or through at least one of the following groups, for example: oxygen atom, nitrogen atom, sulfur atom, silicon atom, phosphorus atom, boron atom, chain structural unit, and aliphatic cyclic group; and where each Ar¹ to Ar⁹ may be further optionally substituted, and the substituents may optionally be hydrogen, alkyl, alkoxy, amino, alkene, alkyne, aralkyl, heteroalkyl, aryl and heteroaryl.

In one aspect, Ar¹ to Ar⁹ may be independently selected from the group consisting of:

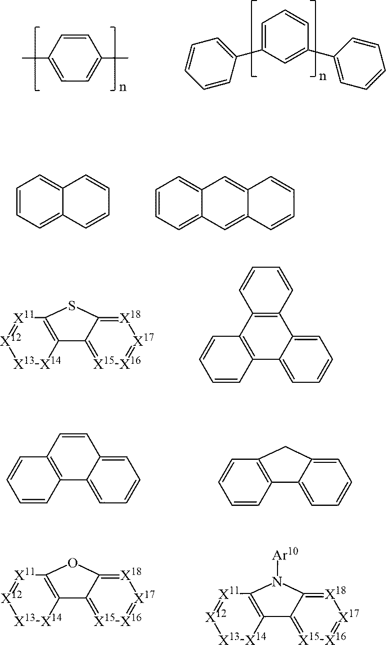

Where n is an integer from 1 to 20; each of X¹¹ to X¹⁸ is CH or N: Ar¹⁰ is defined as the above-mentioned Ar₁.

Additional examples of cyclic aromatic amine-derived compounds may be found in US3567450, US4720432, US5061569, and US3615404.

Examples of suitable HTM compounds are listed below:

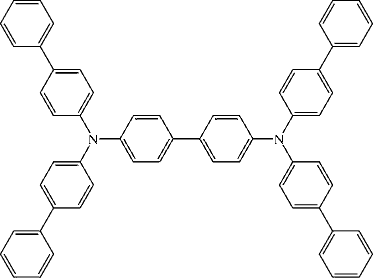 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1]

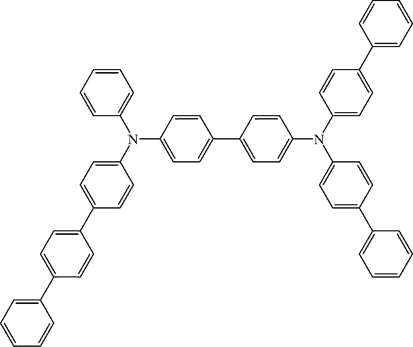 [SMILES: c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

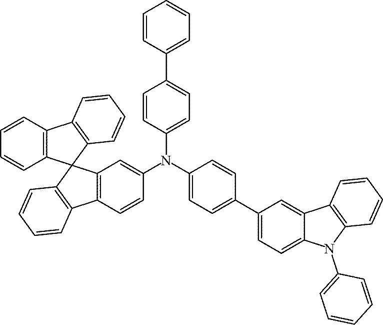 [SMILES: c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

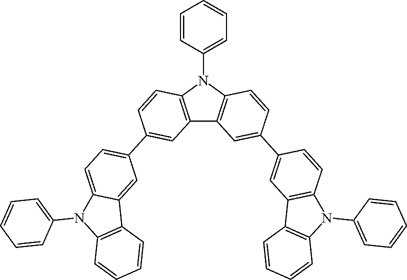 [SMILES: c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1]

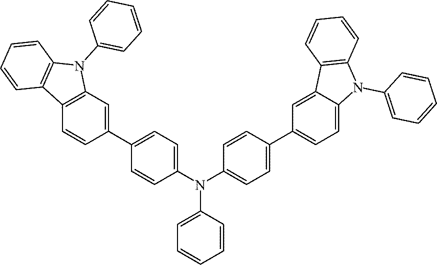 [SMILES: c1ccc(N(c2ccc(-c3ccc4c(c3)c3ccccc3n4-c3ccccc3)cc2)c2ccc(-c3ccc4c5ccccc5n(-c5ccccc5)c4c3)cc2)cc1]

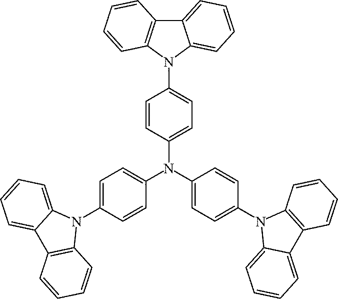 [SMILES: c1ccc2c(c1)c1ccccc1n2-c1ccc(N(c2ccc(-n3c4ccccc4c4ccccc43)cc2)c2ccc(-n3c4ccccc4c4ccccc43)cc2)cc1]

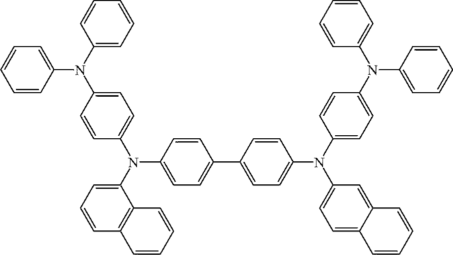 [SMILES: c1ccc(N(c2ccccc2)c2ccc(N(c3ccc(-c4ccc(N(c5ccc(N(c6ccccc6)c6ccccc6)cc5)c5cccc6ccccc56)cc4)cc3)c3ccc4ccccc4c3)cc2)cc1]

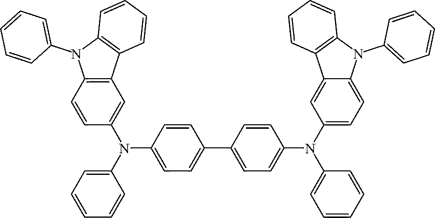 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

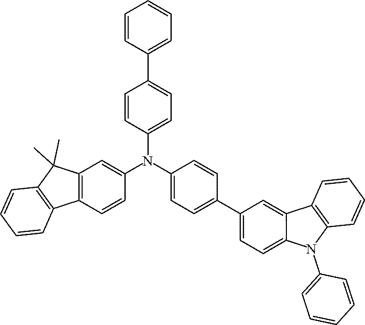 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21]

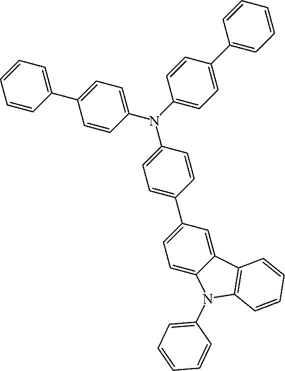 [SMILES: c1ccc(-c2ccc(N(c3ccc(-c4ccccc4)cc3)c3ccc(-c4ccc5c(c4)c4ccccc4n5-c4ccccc4)cc3)cc2)cc1]

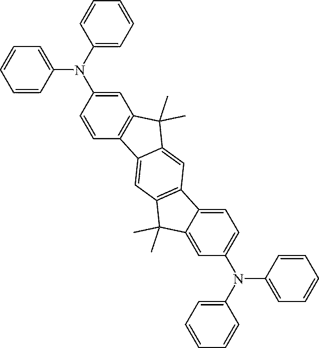 [SMILES: CC1(C)c2cc(N(c3ccccc3)c3ccccc3)ccc2-c2cc3c(cc21)-c1ccc(N(c2ccccc2)c2ccccc2)cc1C3(C)C]

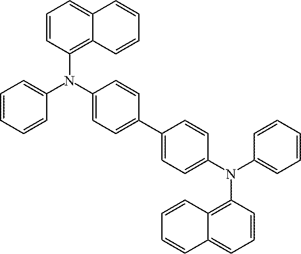 [SMILES: c1ccc(N(c2ccc(-c3ccc(N(c4ccccc4)c4cccc5ccccc45)cc3)cc2)c2cccc3ccccc23)cc1]

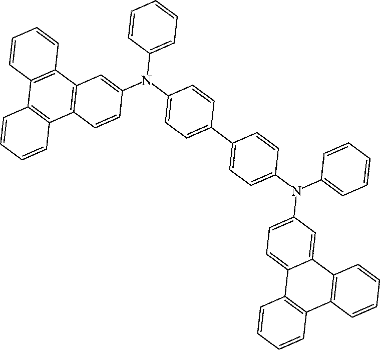 [SMILES: c1ccc(N(c2ccc(-c3ccc(N(c4ccccc4)c4ccc5c6ccccc6c6ccccc6c5c4)cc3)cc2)c2ccc3c4ccccc4c4ccccc4c3c2)cc1]

In yet another aspect, the present disclosure further provides a formulation or ink comprising an organic compound or a mixture as described herein, and at least one organic solvent.

The viscosity and surface tension of the ink are important parameters in printing processes. A suitable ink surface tension is required for the specific substrates and the specific printing methods.

In some embodiments, the surface tension of the ink as described herein at 25° C. is in the range of 19 dynes/cm to 50 dynes/cm; more preferably in the range of 22 dyn/cm to 35 dyne/cm; and most preferably in the range of 25 dyne/cm to 33 dyne/cm.

In some embodiments, the viscosity of the ink as described herein at 25° C. is in the range of from about 1 cps to 100 cps; particularly in the range of 1 cps to 50 cps; more particularly in the range of 1.5 cps to 20 cps; and most particularly in the range of 4.0 cps to 20 cps. The resulting formulation will be particularly suitable for ink-jet printing.

The viscosity can be adjusted by different methods, such as by the selection of appropriate organic solvent and the concentration of the functional materials in the ink. In the ink comprising the above-mentioned metal-organic complexes or polymers as described herein facilitate the adjustment of the printing ink in the appropriate range according to the printing method used. Generally, in the formulation comprising the functional material as described herein, the weight ratio of the functional material ranges from 0.3 wt% to 30 wt%, preferably in the range of 0.5 wt% to 20 wt%, more preferably in the range of 0.5 wt% to 15 wt%, further preferably in the range of 0.5 wt% to 10 wt%, and most preferably in the range of 1 wt% to 5 wt%.

In the formulation as described herein, the at least one organic solvent is selected from aromatics, heteroaromatics, esters, aromatic ketones, aromatic ethers, aliphatic ketones, aliphatic ethers, alicyclic or olefinic compounds, borate, phosphate, or mixtures of two or more of them.

In some embodiments, in the formulation as described herein, the at least one organic solvent is selected from aromatic or heteroaromatic-based solvents.

The aromatic or heteroaromatic-based solvents suitable for the present disclosure include, but not limited to: p-diisopropylbenzene, amylbenzene, tetrahydronaphthalene, cyclohexylbenzene, chloronaphthalene, 1,4-dimethylnaphthalene, 3-isopropylbenzene, p-methylisopropylbenzene, dipentylbenzene, tripentylbenzene, pentyltoluene, o-diethylbenzene, m-diethylbenzene, p-diethylbenzene, 1,2,3,4-tetramethylbenzene, 1,2,3,5-tetramethylbenzene, 1,2,4,5-tetramethylbenzene, butylbenzene, dodecylbenzene, dihexylbenzene, dibutylbenzene, p-diiisopropylbenzene, cyclohexylbenzene, benzylbutylbenzene, dimethylnaphthalene, 3-isopropylbiphenyl, p-methylisopropylbenzene, 1-methylnaphthalene,1,2,4-trichlorobenzene, 4,4-difluorobenzenemethane, 1,2-dimethoxy-4-(1-propenyl) benzene, diphenylmethane, 2-phenylpyridine, 3-phenylpyridine, N-methyldiphenylamine, 4-isopropylbipheny, dichlorodiphenylmethane, 4-(3-phenylpropyl) pyridine, benzyl benzoate, 1,1-bis (3,4-dimethylphenyl) ethane, 2-isopropylnaphthalene, quinoline, isoquinoline, methyl 2-furanecarboxylate, ethyl 2-furanicarboxylate, etc.

The aromatic ketone-based solvents suitable for the present disclosure include, but not limited to: 1-tetrahydronaphthalone, 2-tetrahydronaphthalone, 2-(phenylepoxy)tetrahydronaphthalone, 6-(methoxy)tetrahydronaphthalone, acetophenone, phenylacetone, benzophenone, and derivatives thereof such as 4-methyl acetophenone, 3-methyl acetophenone, 2-methyl acetophenone, 4-methyl propanone, 3-methyl propanone, 2-methyl propanone, etc.

The aromatic ether solvents suitable for the present disclosure include, but not limited to: 3-phenoxytoluene, butoxybenzene, p-anisaldehyde dimethyl acetal, tetrahydro-2-phenoxy-2H-pyran, 1,2-dimethoxy-4-(1-propenyl)benzene, 1,4-benzodioxane, 1,3-dipropylbenzene, 2,5-dimethoxytoluene, 4-ethylphenyl ether, 1,3-dipropoxybenzene, 1,2,4-trimethoxybenzene, 4-(1-propenyl)-1,2-dimethoxybenzene, 1,3-dimethoxybenzene, glycidyl phenyl ether, dibenzyl ether, 4-tert-butyl anisole, trans-anethole, 1,2-dimethoxybenzene, 1-methoxynaphthalene, diphenyl ether, 2-phenoxymethyl ether, 2-phenoxytetrahydrofuran, ethyl-2-naphthyl ether.

In some embodiments, in the formulation as described herein, the at least one organic solvent can be selected from aliphatic ketones, such as, 2-nonanone, 3-nonanone, 5-nonanone, 2-decanone, 2,5-hexanedione, 2,6,8-trimethyl-4-nonanone, fenchone, phoron, isophorone, di-n-amyl ketone, etc; and the at least one organic solvent as described herein can be selected from aliphatic, ethers, such as, dipentyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethylene glycol ethyl methyl ether, triethylene glycol butyl methyl ether, tripropylene glycol dimethyl ether, tetraethylene glycol dimethyl ether, etc.

In some embodiments, in the formulation as described herein, the at least one organic solvent can be selected from: ester-based solvents including alkyl octanoate, alkyl sebacate, alkyl stearate, alkyl benzoate, alkyl phenylacetate, alkyl cinnamate, alkyl oxalate, alkyl maleate, alkyl lactone, alkyl oleate, etc. Particular preferred as octyl octanoate, diethyl sebacate, diallyl phthalate and isononyl isononanoate.

The solvent may be used alone or as mixtures of two or more organic solvents.

In some embodiments, the formulation as described herein comprises an organic compound or a mixture as described herein, and at least one solvent, and can further comprise another organic solvent. Examples of the another organic solvent include, but not limited to: methanol, ethanol, 2-methoxyethanol, dichloromethane, trichloromethane, chlorobenzene, o-dichlorobenzene, tetrahydrofuran, anisole, morpholine, toluene, o-xylene, m-xylene, p-xylene, 1,4 dioxane, acetone, methyl ethyl ketone, 1,2 dichloroethane, 3-phenoxytoluene, 1,1,1-trichloroethane, 1,1,2,2-tetrachloroethane, ethyl acetate, butyl acetate, dimethylformamide, dimethylacetamide, dimethylsulfoxide, tetrahydronaphthalene, decalin, indene, and/or mixtures thereof.

In yet another aspect, the present disclosure further provides the use of the formulation as coatings or printing inks in the preparation of organic electronic devices, particularly preferably by printing or coating processing methods.

Where suitable printing or coating techniques include, but are not limited to, ink-jet printing, gravure printing, typographic printing, screen printing, dip coating, spin coating, blade coating, roller printing, torsion roll printing, planographic printing, flexographic printing, rotary printing, spray printing, brush coating, pad printing, slit die coating, and so on. Preferred techniques are gravure printing, screen printing, and ink-jet printing. Gravure printing and ink-jet printing will be applied in the embodiments of the present disclosure. The solution or dispersion may additionally comprise one or more components, such as surfactants, lubricants, wetting agents, dispersing agents, hydrophobic agents, binders, etc, which are used to adjust the viscosity and film forming properties, or to improve adhesion, etc. For more information about printing technologys and their requirements for solutions, such as solvent, concentration, viscosity, etc, please refer to “Handbook of Print Media: Technologies and Production Methods”, edited by Helmut Kipphan, ISBN 3-540-67326-1.

The preparation methods as described herein, where the formed functional layer has a thickness of 5 nm-1000 nm.

In yet another aspect, the present disclosure further provides the application of an organic compound or a mixture as described herein in organic electronic devices.

In yet another aspect, the present disclosure further provides an organic electronic device comprising an organic compound, or a polymer, or a mixture as described herein.

In some embodiments, the organic electronic device comprising a functional layer, the functional layer comprises an organic compound, or a polymer, or a mixture as described herein.

The organic electronic device may be selected from, but not limited to an organic light emitting diode (OLED), an organic photovoltaic cell (OPV), an organic light emitting electrochemical cell (OLEEC), an organic field effect transistor (OFET), an organic light emitting field effect transistor, an organic laser, an organic spintronic device, a photoelectric sensor, an organic sensor, an organic plasmon emitting diode (OPED), etc., particularly preferably an organic electroluminescent device, such as an OLED, an OLEEC, an organic light emitting field effect transistor.

In some embodiments, the organic electronic device is an organic electroluminescent device comprising an organic compound or a mixture as described herein.

In some embodiments, the organic electronic device is an organic electroluminescent device comprising a light-emitting layer, and the light-emitting layer comprises an organic compound or a mixture as described herein.

In the organic electroluminescent as devices described herein, in particular an OLED, a substrate, an anode, at least one light-emitting layer, and a cathode are included.

The substrate should be opaque or transparent. A transparent substrate could be used to produce a transparent light emitting device (for example: Bulovic et al., Nature 1996, 380, p29, and Gu et al., Appl. Phys. Lett. 1996, 68, p2606). The substrate can be rigid/flexible, and can also be plastic, metal, semiconductor wafer, or glass. Preferably, the substrate has a smooth surface. Particularly desirable are substrates without surface defects. In some embodiments, the substrate is flexible and can be selected from a polymer film or plastic with a glass transition temperature Tg over 150° C., preferably over 200° C., more preferably over 250° C., and most preferably over 300° C. Examples of the suitable flexible substrate includes poly (ethylene terephthalate) (PET) and polyethylene glycol (2,6-naphthalene) (PEN).

The choice of anodes may include a conductive metal, a metal oxide, or a conductive polymer. The anode should be able to easily inject holes into a hole-injection layer (HIL), a hole-transport layer (HTL), or a light emitting layer. In some embodiments, the absolute value of the difference between the work function of the anode and the HOMO energy level of the emitter of the emitting layer, or the HOMO energy level/valence band energy level of the p-type semiconductor material for the hole-inj ection layer (HIL)/hole-transport layer (HTL)/electron-blocking layer (EBL) is less than 0.5 eV, preferably less than 0.3 eV, more preferably less than 0.2 eV. Examples of anode materials may include, but not limited to: Al, Cu, Au, Ag, Mg, Fe, Co, Ni, Mn, Pd, Pt, ITO, aluminum-doped zinc oxide (AZO), etc. Other suitable anode materials are known and can be readily selected for use by one of ordinary skill in the art. The anode material can be deposited using any suitable technique, such as a suitable physical vapor deposition method, including RF magnetron sputtering, vacuum thermal evaporation, e-beam, etc. In some embodiments, the anode is patterned. Patterned conductive ITO substrates are commercially available and can be used to produce the devices as described herein.

The choice of cathode may include a conductive metal and a metal oxide. The cathode should be able to easily inject electrons into the EIL, the ETL, or the directly into the emitting layer. In some embodiments, the absolute value of the difference between the work function of the cathode and the LUMO energy level of the emitter of the emitting layer, or the LUMO energy level/conduction band energy level of the n-type semiconductor material for electron-injection layer (EIL)/electron-transport layer (ETL)/hole-blocking layer (HBL) is less than 0.5 eV, preferably less than 0.3 eV, most preferably less than 0.2 eV. In principle, all materials that may be used as cathodes for OLEDs are possible to apply as cathode materials for the present disclosure. Examples of cathode materials include, but not limited to: Al, Au, Ag, Ca, Ba, Mg, LiF/Al, MgAg alloys, BaF2/Al, Cu, Fe, Co, Ni, Mn, Pd, Pt, ITO, etc. The cathode material can be deposited using any suitable technique, such as the suitable physical vapor deposition method, including RF magnetron sputtering, vacuum thermal evaporation, e-beam, etc.

The OLED may also comprise other functional layers, such as a hole-injection layer (HIL), a hole-transport layer (HTL), an electron-blocking layer (EBL), an electron-injection layer (EIL), an electron-transport layer (ETL), and a hole-blocking layer (HBL). Materials suitable for use in these functional layers are described in detail above and in WO2010135519A1, US20090134784A1, and WO2011110277A1, the entire contents of these three documents are hereby incorporated herein for reference.

In some embodiments, the light-emitting layer of the organic electroluminescent device according to the present disclosure comprises an organic compound or a mixture as described herein.

In some embodiments, the light-emitting layer of the organic electroluminescent device according to the present disclosure can be formed by one of the following methods:

The mixture comprising the organic compound H1 and the organic compound H2 is used as a source and is deposited by vacuum evaporation deposition.

The organic compound H1 and the organic compound H2 are independently used as two separate sources, and are deposited by vacuum evaporation deposition.

The formulation as described herein is deposited by the method of solution processing, especially by printing method.

The light-emitting device as described herein has a light-emitting wavelength between 300 nm and 1000 nm, preferably between 350 nm and 900 nm, more preferably between 400 nm and 800 nm.

In some embodiments, the light-emitting device as described herein has an emission peak wavelength between 500 nm and 800 nm, preferably between 550 nm and 750 nm, and most preferably between 600 nm and 700 nm.

In yet another aspect, the present disclosure further provides the application of organic electroluminescent devices in various electronic equipment, including, but not limited to, display devices, lighting equipment, light sources, sensors, etc.

The present disclosure will be described below in conjunction with the preferred embodiments, but the present disclosure is not limited to the following embodiments. It should be understood that the scope of the present disclosure is covered by the scope of the claims of the present disclosure, and those skilled in the art should understand that certain changes may be made to the embodiments of the present disclosure.

### Specific Embodiment

### 1. Synthesis of Compounds

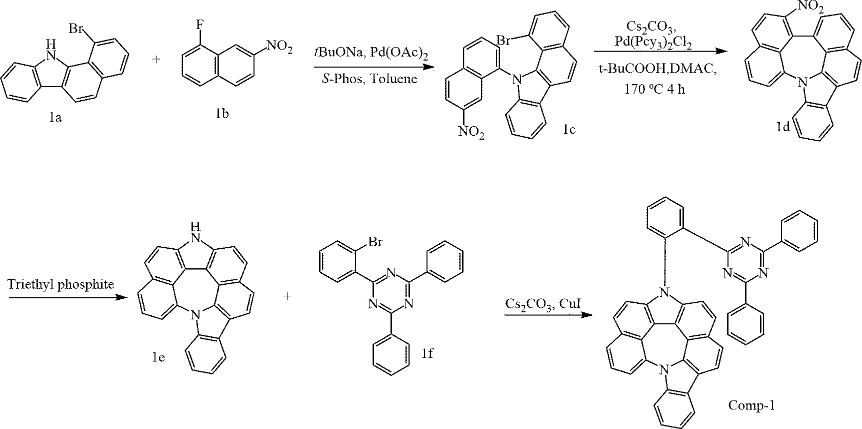

Synthesis of Comp-1:

Compound 1a (20.2 g, 68.4 mmol), compound 1b (13.1 g, 68.4 mmol), palladium acetate (0.77 g, 3.4 mmol), S-Phos (0.77 g, 1.9 mmol), and sodium tert-butoxide (7.9 g, 82 mmol) were dissolved in 200 mL of dry toluene. The resulting mixture was heated to 110° C., and stirred for 6 h under nitrogen atmosphere. After the reaction was completed, the solvent was removed by rotary evaporation, the residue was dissolved in dichloromethane and then washed with water three times. After that, the combined organic phases were separated and purified by silica gel column chromatography (eluent: petroleum ether: dichloromethane = 10:1) to yield 24 g (75% yield) of intermediate 1c.

Intermediate 1c (21 g, 44.9 mmol), caesium carbonate (29.3 g, 89.9 mmol), bis(tricyclohexylphosphine)palladium dichloride (1.7 g, 2.2 mmol), pivalic acid (9.18 g, 89.9 mmol), and 100 mL of N, N-dimethylacetamide were added to a 250 mL three-necked flask. The resulting mixture was heated to 140° C., and stirred for 12 h under nitrogen atmosphere. After the reaction was completed, the mixture was diluted with water. After the filtration, the solid was collected and then purified by silica gel column chromatography (eluent: petroleum ether: dichloromethane=20:1) to yield 16.1 g (93% yield) of Intermediate 1d.

Intermediate 1d (14.9 g, 38.6 mmol) and 350 mL of triethyl phosphite were added to a 500 mL three-necked flask. The resulting mixture was heated to 150° C., and stirred for 12 h under nitrogen atmosphere. After the reaction was completed, the solvent was removed by vacuum distillation, and dissolved in dichloromethane and washed with deionized water many times, then the combined organic phases were separated and purified by silica gel column chromatography (petrol ether: dichloromethane=4:1) to yield 10.9 g (79.8% yield) of intermediate 1e.

Intermediate 1e (10 g, 28.2 mmol), compound 1f(11 g, 28.2 mmol), cesium carbonate (11 g, 33.8 mmol), and copper iodide (0.5 g, 2.8 mmol) were dissovled in 100 mL of dry toluene under nitrogen atmosphere. The resulting mixture was heated to 110° C., and stirred for 6 h. After the reaction was completed, the solvent was removed by rotary evaporation, the residue was then dissolved in dichloromethane and washed with water three times. After that, the combined organic phases were separated and purified by silica gel column chromatography (eluent: petroleum ether: dichloromethane = 20:1) to yield 14 g (75% yield) of compound-1 (solid powder).

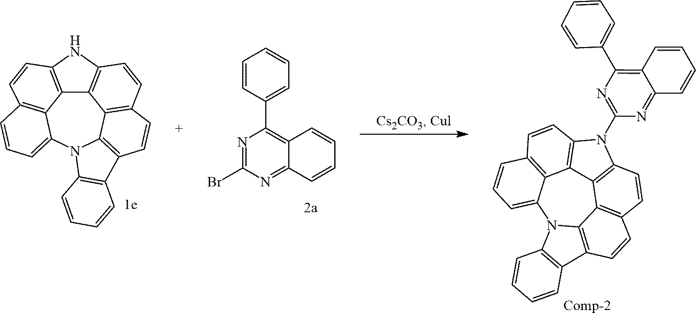

Synthesis of Comp-2:

Intermediate1e (10 g, 28.2 mmol), compound 2a (8 g, 28.2 mmol), cesium carbonate (11 g, 33.8 mmol), and copper iodide (0.5 g, 2.8 mmol) were dissovled in 100 mL of dry toluene under nitrogen atmosphere. The resulting mixture was heated to 110° C., and stirred for 6 h. After the reaction was completed, the solvent was removed by rotary evaporation, the residue was dissolved in dichloromethane and washed with water three times. After that, the combined organic phases were separated and purified by silica gel column chromatography (eluent: petroleum ether: dichloromethane = 15:1) to yield 12.2 g (77.4% yield) of compound-2 (solid powder).

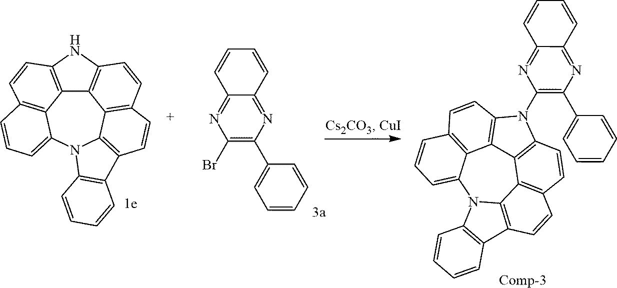

Synthesis of Comp-3:

Intermediate 1e (10 g, 28.2 mmol), compound 3a (8 g, 28.2 mmol), cesium carbonate (11 g, 33.8 mmol), and copper iodide (0.5 g, 2.8 mmol) were dissolved in 100 mL of dry toluene under nitrogen atmosphere. The resulting mixture was heated to 110° C., and stirred for 6 h. After the reaction was completed, the solvent was removed by rotary evaporation, the residue was dissolved in dichloromethane and washed with water three times. After that, the combined organic phases were separated and purified by silica gel column chromatography (eluent: petroleum ether: dichloromethane = 15:1) to yield 11.3 g (72% yield) of compound-3 (solid powder).

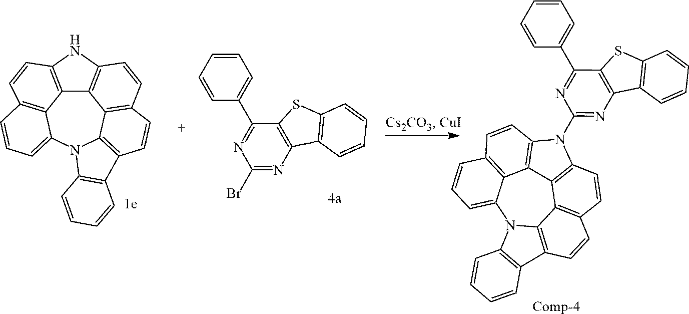

Synthesis of Comp-4:

Intermediate 1e (10 g, 28.2 mmol), compound 4a (9.6 g, 28.2 mmol), cesium carbonate (11 g, 33.8 mmol), and copper iodide (0.5 g, 2.8 mmol) were dissolved in 100 mL of dry toluene under nitrogen atmosphere. The resulting mixture was heated to 110° C., and stirred for 12 h. After the reaction was completed, the solvent was removed by rotary evaporation, the residue was dissolved in dichloromethane and washed with water three times. After that, the combined organic phases were separated and purified by silica gel column chromatography (eluent: petroleum ether: dichloromethane = 20:1) to yield 11.9 g (66% yield) of compound-4 (solid powder).

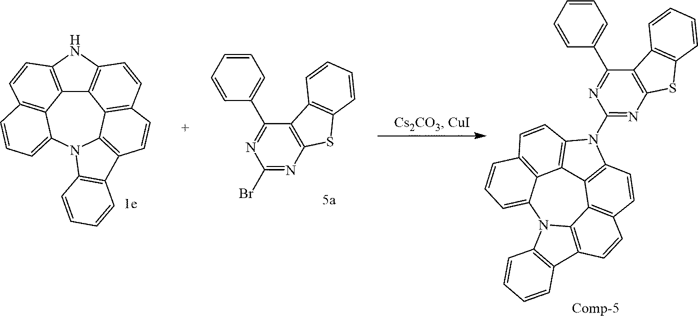

Synthesis of Comp-5:

Intermediate 1e (10 g, 28.2 mmol), compound 5a (9.6 g, 28.2 mmol), cesium carbonate (11 g, 33.8 mmol), and copper iodide (0.5 g, 2.8 mmol) were dissolved in 100 mL of dry toluene under nitrogen atmosphere. The resulting mixture was heated to 110° C., and stirred for 12 h. After the reaction was completed, the solvent was removed by rotary evaporation, the residue was dissolved in dichloromethane and washed with water three times. After that, the combined organic phases were separated and purified by silica gel column chromatography (eluent: petroleum ether: dichloromethane = 20:1) to yield 12.4 g (69% yield) of compound-5 (solid powder).

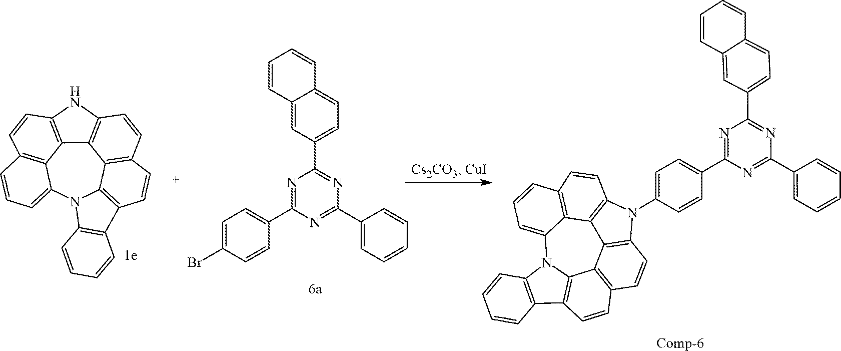

Synthesis of Comp-6:

Intermediate 1e (10 g, 28.2 mmol), compound 6a (12.4 g, 28.2 mmol), cesium carbonate (11 g, 33.8 mmol), and copper iodide (0.5 g, 2.8 mmol) were dissolved in 100 mL of dry toluene under nitrogen atmosphere. The resulting mixture was heated to 110° C., and stirred for 12 h. After the reaction was completed, the solvent was removed by rotary evaporation, the residue was dissolved in dichloromethane and washed with water three times. After that, the combined organic phases were separated and purified by silica gel column chromatography (eluent: petroleum ether: dichloromethane = 20:1) to yield 15.3 g (76% yield) of compound-6 (solid powder).

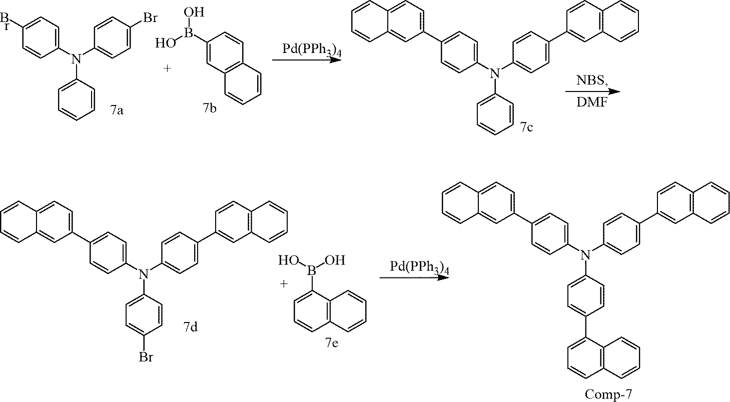

Synthesis of Comp-7:

Compound 7a (20.2 g, 50 mmol), compound 7b (17.2 g, 100 mmol), tetrakis(triphenylphosphine) palladium (3.5 g, 3 mmol), tetrabutylammonium bromide (8.1 g, 25 mmol), and sodium hydroxide (4 g, 100 mmol) were dissolved in the mixture of 200 mL of dry toluene and 50 mL of deionized water under nitrogen atmosphere. The resulting mixture was heated to 110° C., and stirred for 24 h. After the reaction was completed, the solvent was removed by rotary evaporation, the residue was dissolved in dichloromethane and washed with water three times. After that, the combined organic phases were separated and purified by silica gel column chromatography (eluent: petroleum ether) to yield 18.7 g (75% yield) of intermediate 7c.

Intermediate 7c (14.9 g, 30 mmol) was dissolved in 100 mL of N, N-dimethylformamide, then a N,N-dimethylformamide solution of NBS (30 mmol) was added dropwise under ice bath, and the reaction mixture was stirred and reacted for 12 h in dark. After the reaction was completed, the mixture was poured into 300 mL of water, and filtered, then the residue was recrystallized to yield 17.3 g (90% yield) of intermediate 7d.

Intermediate 7d (34.4 g, 20 mmol), compound 7e (11.5 g, 20 mmol), tetrakis(triphenylphosphine) palladium (0.7 g, 0.6 mmol), tetrabutylammonium bromide (3.2 g, 10 mmol), and sodium hydroxide (1.6 g, 40 mmol) were dissolved in the mixture of 200 mL of dry toluene and 50 mL of deionized water under nitrogen atmosphere. The resulting mixture was heated to 110° C., and stirred for 12 h. After the reaction was completed, the solvent was removed by rotary evaporation, then the product was dissolved in dichloromethane and washed with saturated brine three times. After that, the combined organic phases were separated and purified by silica gel column chromatography (eluent: dichloromethane: petroleum ether=1:10) to yield 18.7 g (85% yield) of compound-7 (solid powder).

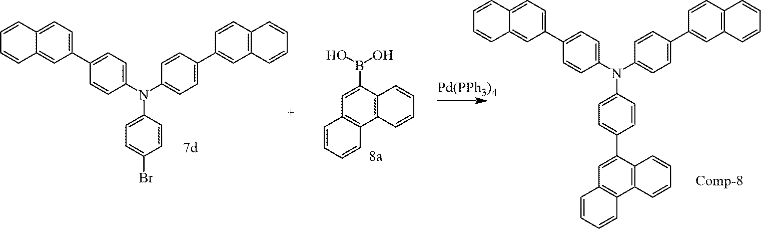

Synthesis of Comp-8:

Intermediate 7d (11.5 g, 20 mmol), compound 8a (44.4 g, 20 mmol), tetrakis(triphenylphosphine) palladium (0.7 g, 0.6 mmol), tetrabutylammonium bromide (3.2 g, 10 mmol), sodium hydroxide (1.6 g, 40 mmol) were dissolved in the mixture of toluene (80 mL) and water (10 mL) under nitrogen atmosphere. The resulting mixture was heated to 110° C., and stirred for 12 h. After the reaction was completed, the solvent was removed by rotary evaporation, the residue was dissovled in dichloromethane and washed with water three times. After that, the combined organic phases were separated and purified by silica gel column chromatography (eluent: dichloromethane: petroleum ether = 1:10) to yield 21.8 g (85% yield) of compound-8 (solid powder).

### 2.Energy Structure of Organic Compound

The energy level of the organic material can be calculated by quantum computation, for example, using TD-DFT (time-dependent density functional theory) by Gaussian09W (Gaussian Inc.), the specific simulation methods of which can be found in WO2011141110. Firstly, the molecular geometry is optimized by semi-empirical method “Ground State/Semi-empirical/Default Spin/AM1” (Charge 0/Spin Singlet), and then the energy structure of organic molecules is calculated by TD-DFT (time-dependent density functional theory) “TD-SCF/DFT/Default Spin/B3PW91” and the basis set “6-31G (d)” (Charge 0/Spin Singlet). The HOMO and LUMO levels are calculated using the following calibration formula, where S1 and T1 are used directly.

$$\text{HOMO} \left(\text{eV}\right) \text{=} \left(\left(\text{HOMO} \left(\text{G}\right) \times 27.212\right) \text{-0} \text{.9899}\right) / 1.1206$$

$$\text{LUMO} \left(\text{eV}\right) \text{=} \left(\left(\text{LUMO} \left(\text{G}\right) \times 27.212\right) \text{-2} \text{.0041}\right) / 1.385$$

Where HOMO(G) and LUMO(G) are the direct calculation results of Gaussian 09W, in units of Hartree. The results are shown in Table 1:

TABLE 1

| Compound | Homo [eV] | Lumo [eV] | ΔLumo [eV] | ΔHomo [eV] | T1 [eV] | S1-T1 |
|---|---|---|---|---|---|---|
| B | -5.39 | -2.39 | 0.23 | 0.15 | 2.35 | 0.81 |
| Comp-1 | -5.36 | -2.87 | 0.03 | 0.15 | 2.35 | 0.24 |
| Comp-2 | -5.39 | -3.03 | 0.61 | 0.09 | 2.26 | 0.22 |
| Comp-3 | -5.44 | -3.10 | 0.66 | 0.13 | 2.27 | 0.12 |
| Comp-4 | -5.40 | -3.00 | 0.54 | 0.11 | 2.29 | 0.23 |
| Comp-5 | -5.43 | -2.86 | 0.38 | 0.10 | 2.26 | 0.50 |
| Comp-6 | -5.44 | -3.00 | 0.03 | 0.16 | 2.33 | 0.33 |
| Comp-7 | -5.34 | -2.42 | 0.03 | 0.73 | 2.46 | 0.77 |
| Comp-8 | -5.34 | -2.42 | 0.03 | 0.70 | 2.46 | 0.78 |

### 3. Preparation and Measurement of OLED Devices

The preparation process of the OLEDs will be described in detail with reference to specified examples below. The structure of the red-emitting OLED is as follows: ITO/HI/HT-1/HT-2/EML/ET: Liq/Liq/Al.

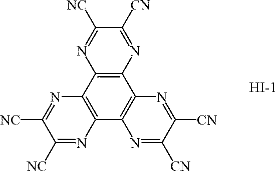

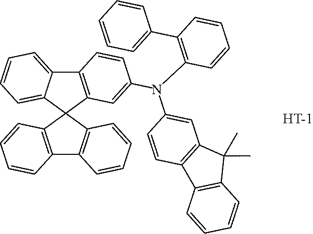 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

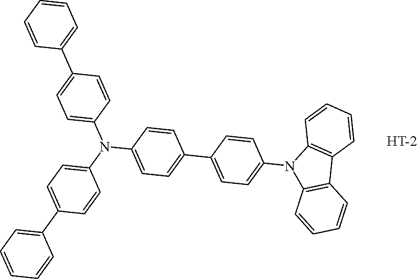 [SMILES: c1ccc(-c2ccc(N(c3ccc(-c4ccccc4)cc3)c3ccc(-c4ccc(-n5c6ccccc6c6ccccc65)cc4)cc3)cc2)cc1]

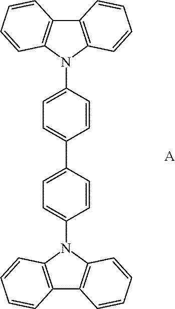 [SMILES: c1ccc2c(c1)c1ccccc1n2-c1ccc(-c2ccc(-n3c4ccccc4c4ccccc43)cc2)cc1]

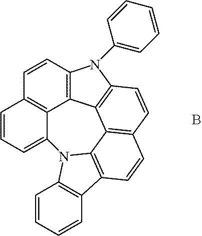 [SMILES: c1ccc(-n2c3ccc4cccc5c4c3c3c4c(ccc6c7ccccc7n5c64)ccc32)cc1]

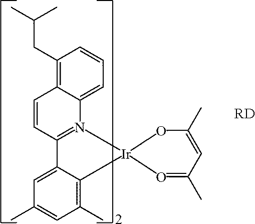

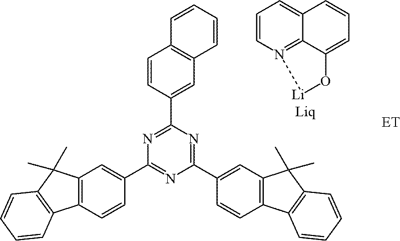

The preparation steps are as follows:

a. Cleaning of the ITO (Indium Tin Oxide) conductive glass substrate: the substrates are washed with various solvents (such as one or more of chloroform, ketone, or isopropyl alcohol), and then treated with UV and ozone.

b. The materials were vacuum-deposited in high vacuum (1×10⁻⁶ mbar) to form the following layers: HI (30 nm), HT-1 (60 nm), HT-2 (10 nm), host material: 3% RD (40 nm), ET: Liq (50: 50, 30 nm), Liq (1 nm), and Al (100 nm). After cleaning, the resultant ITO substrate was mounted on a vacuum deposition apparatus, compound HI-1 was vacuum-deposited on the anode to form a hole-injection layer having a thickness of 30 nm, then compound HT-1 and compound HT-2 were vacuum-deposited sequentially on the hole-inj ection layer to form two hole-transport layers having a thickness of 60 nm and 10 nm, respectively. Comp-1 (host) and compound RD (dopant) were placed in two different evaporation sources, and co-deposited on the hole-transport layer at a weight ratio of 100:3 to form a light emitting layer having a thickness of 40 nm. Subsequently, compound ET and Liq were placed in two different evaporation sources, and co-deposited on the light emitting layer at a weight ratio of 50:50 to form an electron-transport layer having a thickness of 30 nm. Liq was then deposited on the electron-transport layer to form an electron-injection layer having a thickness of 1 nm and Al was deposited on the electron-injection layer to form a cathode having a thickness of 100 nm.

c. Encapsulation: encapsulating the device in a nitrogen-regulated glove box with UV curable resin.

Device Examples 2-11 and Comparative Example 1-4 were implemented in the same way as device example 1, except that corresponding compounds and the different co-hosts shown in Table 1 were utilized instead of comp-1 in forming a light emitting layer. The co-host refers to that two compounds were respectively placed in different evaporation sources, and control the weight ratio of materials during the co-evaporation.

The current-voltage and luminescence (IVL) characteristics of the red-emitting OLEDs were studied. The current efficiency and device lifetime were recorded in Table 2. The lifetime of each Device Examples 1-11 and Comparative Example 2-4 is a relative value of that of Comparative Example 1.

TABLE 2

| OLED Device | Host | Current efficiency CE@1000nits (cd/A) | T95@1000nits |
|---|---|---|---|
| Device Example 1 | comp-1 | 24.7 | 273% |
| Device Example 2 | comp-2 | 23.6 | 252% |
| Device Example 3 | comp-3 | 24.1 | 267% |
| Device Example 4 | comp-4 | 24.0 | 242% |
| Device Example 5 | comp-5 | 23.9 | 263% |
| Device Example 6 | comp-6 | 23.5 | 248% |
| Device Example 7 | comp-1: comp-7=7:3 | 25.4 | 351% |
| Device Example 8 | comp-3: comp-7=7:3 | 24.9 | 319% |
| Device Example 9 | comp-6: comp-7=7:3 | 26.1 | 406% |
| Device Example 10 | comp-1: comp-8=7:3 | 25.1 | 344% |
| Device Example 11 | comp-6: comp-8=7:3 | 25.7 | 380% |
| Comparative Example 1 | A | 16.7 | 100% |
| Comparative Example 2 | B | 14.0 | 72% |
| Comparative Example 3 | B: comp-7=7:3 | 15.5 | 79% |
| Comparative Example 4 | B: comp-8=7:3 | 15.2 | 84% |

Table 2 shows that the luminescence efficiency and lifetime of the device embodiments 1-11 are obviously improved compared with the comparative example 1. Compared with compound B of the comparative example 2, the introduction of nitrogen heterocycles can enhance the electron transport ability of the material, and improve the carrier balance, thus improving the device efficiency and lifetime. It means that the luminescence efficiency and lifetime of OLEDs prepared with the organic compound of the present disclosure are greatly improved.

The technical features of the above-described embodiments can be combined in any ways. For the sake of brevity, not all possible combinations of the technical features of the above-described embodiments have been described. However, as long as there are no contradictions in the combination of these technical features, they should be considered to be within the scope of this specification.

What described above are several embodiments of the present disclosure, and they are specific and in detail, but not intended to limit the scope of the present disclosure. It will be understood that improvements can be made without departing from the concept of the present disclosure, and all these modifications and improvements are within the scope of the present disclosure. The scope of the present disclosure shall be subject to the appended claims.

What is claimed is:

1. An organic compound, comprising a structure of formula (I): 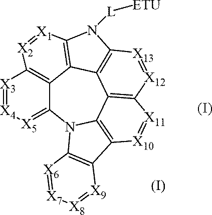
wherein:
each of X₁ to X₁₃ independently represents CR₁, and when the adj acent two CR_{1S} can be fused to form a ring;
ETU is a structure of formula (I-1) or (I-2); 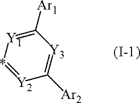 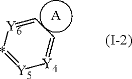
each “*” represents a site connecting L;
A is a C₆-C₃₀ aromatic ring, a C₅-C₃₀ heteroaromatic ring, or any combination thereof;
each of Y₁ to Y₆ independently represents N or CR₂, at least one of Y₁ to Y₃ is N, and at least one of Y₄ to Y₆ is N;
each of R₁ to R₂ at each occurrence is independently selected from the group consisting of -H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, a CF₃ group, Cl, Br, F, a cross-linkable group, a substituted/unsubstituted aromatic or heteroaromatic group containing 5 to 40 ring atoms, an aryloxy or heteroaryloxy group containing 5 to 40 ring atoms, and any combination thereof;
L is a linking group selected from the group consisting of a single bond, a C₆-C₆₀ arylene group, a C₂-C₆₀ heteroaromatic group, a C₃-C₆₀ aliphatic ring, a C₆-C₆₀ fused aromatic ring, and any combination thereof;
each of Ar₁ and Ar₂ is independently selected from the group consisting of a substituted/unsubstituted aromatic or heteroaromatic group containing 5 to 40 ring atoms, an aryloxy or heteroaryloxy group containing 5 to 40 ring atoms, and any combination thereof, wherein one or more Ar₁-Ar₂ may form a monocyclic or polycyclic aliphatic or aromatic ring system with each other and/or with the rings bonded thereto.

2. The organic compound according to claim 1, wherein ETU is selected from the following structures: 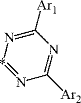 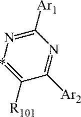 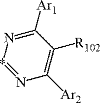 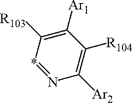 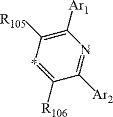 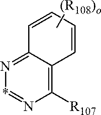 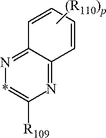 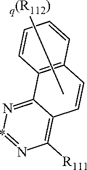 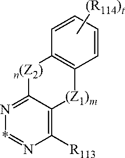 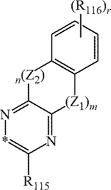
wherein,
each of Z₁ and Z₂ is independently selected from S, O, CR₁₁₇R₁₁₈, or NR₁₁₉;
R₁₀₁ to R₁₁₉ can be the same or different groups with the same meaning as R₁;
o, p, t, and r are integers from 0 to 4; q is an integer from 0 to 6; each of m and n is 0 or 1, and m+n=1;
each “*” represents a site connecting L; each of Ar₁ and Ar₂ is independently selected from the group consisting of a substituted/unsubstituted aromatic or heteroaromatic group containing 5 to 40 ring atoms, an aryloxy or heteroaryloxy group containing 5 to 40 ring atoms, and any combination thereof, wherein one or more Ar₁-Ar₂ may form a monocyclic or polycyclic aliphatic or aromatic ring system with each other and/or with the rings bonded thereto.

3. The organic compound according to claim 1, wherein Ar₁, Ar₂, and L are independently selected from one or combinations of more than one of the following groups: 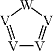 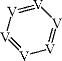 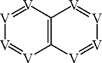 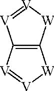 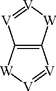 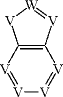 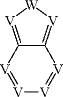 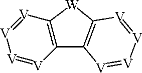 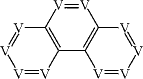 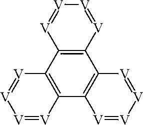
wherein:
V in multiple occurrences, is independently selected from CR₃ or N;
W in multiple occurrences, is independently selected from CR₄R₅, SiR₆R₇, NR₈, C(═O), S, or O;
R₃ to R₈ at each occurrence are independently selected from the group consisting of -H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, a CF₃ group, Cl, Br, F, a cross-linkable group, a substituted/unsubstituted aromatic or heteroaromatic group containing 5 to 40 ring atoms, an aryloxy or heteroaryloxy group containing 5 to 40 ring atoms, and any combination thereof, wherein one or more R₃-R₈ may form a ring system with each other and/or with the groups bonded thereto.

4. The organic compound according to claim 2, wherein Ar₁, Ar₂, and L are independently selected from one or combinations of more than one of the following groups: 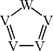 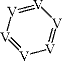 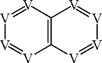 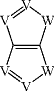 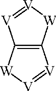 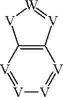 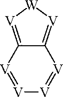 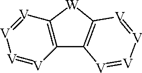 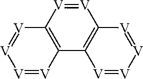 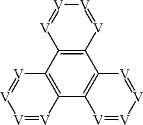
wherein:
V in multiple occurrences, is independently selected from CR₃ or N;
W in multiple occurrences, is independently selected from CR₄R₅, SiR₆R₇, NR₈, C(═O), S, or O;
R₃ to R₈ at each occurrence are independently selected from the group consisting of -H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, a CF₃ group, Cl, Br, F, a cross-linkable group, a substituted/unsubstituted aromatic or heteroaromatic group containing 5 to 40 ring atoms, an aryloxy or heteroaryloxy group containing 5 to 40 ring atoms, and any combination thereof, wherein one or more R₃-R₈ may form a ring system with each other and/or with the groups bonded thereto.

5. A mixture, comprising an organic compound H1 and organic compound H2, wherein the organic compound H1 is selected from an organic compound according to claim 1, the organic compound H2 is another organic functional material, and is selected from one or more of the following: a hole-injection material, a hole-transport material, an electron-transport material, an electron-injection material, an electron-blocking material, a hole-blocking material, an emitting material, a host material, or an organic dye.

6. The mixture according to claim 5, wherein the organic compound H2 comprises a structure of formula (II): 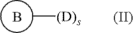
wherein, B is selected from a C₁-C₃₀ substituted/unsubstituted alkyl group, a C₃-C₃₀ substituted/unsubstituted cycloalkyl group, or a C₅-C₆₀ substituted/unsubstituted aromatic hydrocarbon or aromatic heterocyclic group; D is an electron-rich group; s is an integer from 1 to 6.

7. The mixture according to claim 6, wherein the electron-rich group D comprises any one of the following groups or any combination thereof: 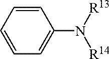 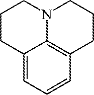 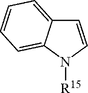 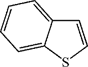 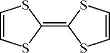 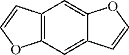 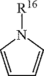 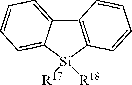 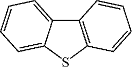 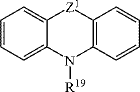 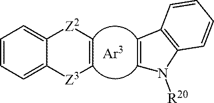
wherein,
Ar³ represents a C₅-C₄₀ aromatic or heteroaromatic group;
Z¹, Z², Z³ are independently selected from a single bond, CR²¹R²², SiR²³R²⁴, NR²⁵, O, C(═O), S, S═O, or SO₂, and Z² and Z³ are not single bonds at the same time;
R¹³ to R²⁵ are independently selected from the group consisting of -H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, a CF₃ group, Cl, Br, F, a cross-linkable group, a substituted/unsubstituted aromatic or heteroaromatic group containing 5 to 40 ring atoms, an aryloxy or heteroaryloxy group containing 5 to 40 ring atoms, and any combination thereof, wherein one or more R¹³-R²⁵ may form a ring system with each other and/or with the groups bonded thereto.

8. The mixture according to claim 5, wherein the mixture further comprises an emitting material, and the emitting material is selected from the group consisting of a singlet emitting material, a triplet emitting material, and a TADF material.

9. The mixture according to claim 6, wherein the mixture further comprises an emitting material, and the emitting material is selected from the group consisting of a singlet emitting material, a triplet emitting material, and a TADF material.

10. An organic electronic device, comprising a functional layer, wherein the functional layer comprises the organic compound according to claim 1.

11. The organic electronic device according to claim 10, wherein the organic electronic device is selected from an organic light emitting diode, an organic photovoltaic cell, an organic light emitting electrochemical cell, an organic field effect transistor, an organic light emitting field effect transistor, an organic laser, an organic spintronic device, a photoelectric sensor, an organic sensor, or an organic plasmon emitting diode.

12. The organic electronic device according to claim 11, wherein the organic electronic device is an organic electroluminescent device comprising the organic compound.

13. An organic electronic device, comprising a functional layer, wherein the functional layer comprises the mixture according to claim 5.

14. The organic electronic device according to claim 13, wherein the organic electronic device is selected from an organic light emitting diode, an organic photovoltaic cell, an organic light emitting electrochemical cell, an organic field effect transistor, an organic light emitting field effect transistor, an organic laser, an organic spintronic device, a photoelectric sensor, an organic sensor, or an organic plasmon emitting diode.

15. The organic electronic device according to claim 14, wherein the organic electronic device is an organic electroluminescent device comprising the mixture.
